(19) United States
(12) Patent Application Publication
(10) Pub. No.: US 2007/0027150 A1
(43) Pub. Date: Feb. 1, 2007

(54) 2-Amino-quinazolin-5-ones

(76) Inventors: Cornelia R. Bellamacina, Castro Valley, CA (US); Abran Costales, El Cerrito, CA (US); Brandon M. Doughan, Eugene, OR (US); Susan Fong, Richmond, CA (US); Zhenhai Gao, Hercules, CA (US); Thomas Hendrickson, Encinitas, CA (US); Barry H. Levine, Lafayette, CA (US); Xiaodong Lin, Walnut Creek, CA (US); Timothy D. Machajewski, Martinez, CA (US); Christopher McBride, Oakland, CA (US); William R. Antonios-McCrea, Berkeley, CA (US); Maureen McKenna, Pinole, CA (US); Kris G. Mendenhall, Concord, CA (US); Alice C. Rico, Castro Valley, CA (US); Cynthia M. Shafer, Moraga, CA (US); X. Michael Wang, Livermore, CA (US); Yi Xia, Foster City, CA (US); Yasheen Zhou, Moraga, CA (US)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(21) Appl. No.: 11/404,372

(22) Filed: Apr. 14, 2006

Related U.S. Application Data
(60) Provisional application No. 60/671,662, filed on Apr. 14, 2005.

(51) Int. Cl. A61K 31/5377; A61K 31/517; C07D 413/02; C07D 403/02

(52) U.S. Cl. 514/234.2; 544/123; 544/284; 514/252.17; 514/255.05; 514/266.2; 514/266.22

(57) ABSTRACT

2-Amino-quinazolin-5-one compounds, stereoisomers, tautomers, pharmaceutically acceptable salts, and prodrugs thereof; compositions that include a pharmaceutically acceptable carrier and one or more of the 2-amino-quinazolin-5-one compounds, either alone or in combination with at least one additional therapeutic agent. Methods of using the 2-amino-quinazolin-5-one compounds, either alone or in combination with at least one additional therapeutic agent, in the prophylaxis or treatment of cell proliferative diseases.

### CROSS REFERENCE TO RELATED APPLICATIONS

This application claims the benefit under 35 U.S.C. 119(e) to co-pending provisional application U.S. Ser. No. 60/671,662 filed on Apr. 14, 2005 which is incorporated herein by reference in its entirety.

### FIELD OF THE INVENTION

The present invention relates to new 2-amino-quinazolin-5-one compounds, their stereoisomers, tautomers, pharmaceutically acceptable salts, and prodrugs thereof; to compositions containing 2-amino-quinazolin-5-one compounds and a pharmaceutical acceptable carrier; and to the uses of the compounds and compositions, either alone or in combination with at least one additional therapeutic agent, in the prophylaxis or treatment of cell proliferative diseases.

### BACKGROUND OF THE INVENTION

Heat shock or stress dramatically increases cellular production of several classes of highly conserved chaperone proteins, commonly known as heat-shock proteins (HSPs). These chaperones, including the members of the HSP60, HSP70 and HSP90 families, are ATP-dependent molecules that facilitate/ensure proper client protein (e.g. protein that requires interaction with the chaperones for its activity and stability) folding, prevent non-specific aggregations, and maintain active protein conformations.

The HSP90 family, comprised of HSP90 α and β, Grp94 and TRAP-1, is one of the most abundant cellular proteins, accounting for 1-2% of total proteins in a mammalian cell under normal conditions. HSP90 is unique among cellular chaperones in that it is not required for general co-translational protein folding, but instead is dedicated to a subset of signaling molecules that are frequently mutated or over-expressed in cancer cells. Many of these client proteins, including the mutated p53, Bcr-Abl, Raf-1, Akt, ErbB2, and steroid receptors etc, are well-known and established cancer drug targets. The association with HSP90 ensures that these otherwise unstable oncoproteins function properly in multiple signaling pathways that are essential in maintaining the unregulated growth and the malignant phenotypes of tumors.

Crystallographic studies have revealed the existence of an unconventional low affinity ATP binding cleft at their N-terminal domain that is well conserved among the four HSP90 family members. ATP binding and hydrolysis play an essential role in the regulation of chaperone functions. The occupancy of the ATP binding site by the ansamycin antibiotics geldanamycin (GM) and herbimycin A (HA), as well as the structurally unrelated fungal metabolite radicicol, inhibits the intrinsic ATPase activity of HSP90, and blocks the ATP/ADP-regulated association-dissociation cycles between HSP90 and client proteins. Consequently, ATP-competitive HSP90 inhibitors induce destabilization and eventual ubiquitin-dependent degradation of multiple client proteins. Depending on cellular contexts, HSP90 inhibitors effectively cause growth arrest, differentiation, or apoptosis of tumor cells both in vitro and in vivo.

HSP90 is overexpressed (about 2-20 fold) in multiple tumor types as a result of oncogenic transformation (e.g. accumulation of mutated proteins) and cellular stress (e.g. low pH and lack of nutrients). Cancer cells are very adaptive to hostile microenvironments and are capable of acquiring drug resistance, in part due to their inherent genetic instability and plasticity. Moreover, most forms of cancer are polygenic and harbor multiple signaling aberrations. Hence, a need exists for inhibitors of HSP90 to combat a variety of hard-to-treat tumors by disrupting concurrently a wide range of oncogenic pathways.

### SUMMARY OF THE INVENTION

In one aspect of the present invention, new 2-amino-quinazolin-5-one compounds, their pharmaceutically acceptable salts, and prodrugs thereof are provided. The 2-amino-quinazolin-5-one compounds, pharmaceutically acceptable salts, and prodrugs are HSP90 inhibitors and are useful in treating cellular proliferation diseases.

In one embodiment, the 2-amino-quinazolin-5-one compounds have formula (I):

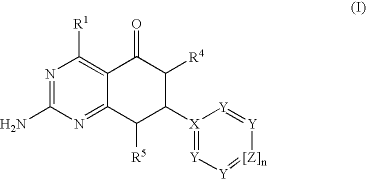

or a stereoisomer, tautomer, pharmaceutically acceptable salt, or prodrug thereof, wherein

n is 0 or 1;

wherein when n is 1, X is C, Y is at each position independently selected from CQ¹ and N, and Z is selected from CR² and N, and

wherein when n is 0, X is C or N, Y is at each position independently selected from CQ¹, N, NQ², O, and S;

wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;

wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;

wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkylamino,
(9) substituted or unsubstituted aryl,
(10) substituted or unsubstituted heteroaryl, and
(11) substituted or unsubstituted heterocyclyl;

wherein R² is selected from the group consisting of
(1) hydrogen,
(2) halogen,
(3) substituted or unsubstituted C₁-C₆ alkyl, and
(4) —OR³, —SR³, or —N(R³)₂;

wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;

wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino; and

with the proviso that when R¹ is methyl, and R⁴ and R⁵ are hydrogen, then X, Y, Z, and n together do not form an unsubstituted phenyl or furan-2-yl ring, and

with the proviso that when R¹, R⁴, and R⁵ are hydrogen, then X, Y, Z, and n together do not form a furan-2-yl, thien-2-yl, or phenyl ring wherein said ring is unsubstituted or substituted with one, two, or three substituents independently selected from the group consisting of C₁-C₆ alkyl, C₁-C₆ alkoxy, amino, alkylamino, dialkylamino, hydroxyl, and halo.

In another aspect, provided are also pharmaceutical compositions comprising a pharmaceutically acceptable carrier and one or more 2-amino-quinazolin-5-one compounds, either alone or in combination with at least one additional therapeutic agent. In one embodiment, the compositions comprise a pharmaceutically acceptable carrier and a compound having formula (V)

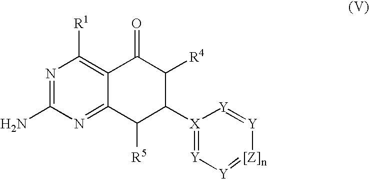

or a stereoisomer, tautomer, pharmaceutically acceptable salt, or prodrug thereof, wherein

n is 0 or 1;

wherein when n is 1, X is C, Y is at each position independently selected from CQ¹ and N, and Z is selected from CR² and N, and

wherein when n is 0, X is C or N, Y is at each position independently selected from CQ¹, N, NQ², O, and S;

wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;

wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;

wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkylamino,
(9) substituted or unsubstituted aryl,
(10) substituted or unsubstituted heteroaryl, and
(11) substituted or unsubstituted heterocyclyl;

wherein R² is selected from the group consisting of
(1) hydrogen,
(2) halogen,
(3) substituted or unsubstituted C₁-C₆ alkyl, and
(4) —OR³, —SR³, or —N(R³)₂;

wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;

wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino. In another aspect, the present invention provides methods for treating proliferative diseases in a human or animal subject in need of such treatment comprising administering to said subject an amount of a compound or composition of formula (I) or (V) effective to reduce or prevent cellular proliferation in the subject.

In another aspect, the present invention provides methods for treating proliferative diseases in a human or animal subject in need of such treatment, comprising administering to said subject an amount of a compound or composition of formula (I) or (V) effective to reduce or prevent cellular proliferation in the subject in combination with at least one additional agent for the treatment of cancer.

The compounds of the invention are useful in the treatment of cancers, including, for example, lung and bronchus; prostate; breast; pancreas; colon and rectum; thyroid; stomach; liver and intrahepatic bile duct; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-hodgkin lymphoma; melanoma; and villous colon adenoma.

The invention further provides additional compounds, compositions, kits, methods of use, and methods of manufacture as described in the detailed description of the invention.

### DETAILED DESCRIPTION

In one aspect of the present invention, new 2-amino-quinazolin-5-one compounds, their stereoisomers, tautomers, pharmaceutically acceptable salts, and prodrugs thereof are provided. The 2-amino-quinazolin-5-one compounds, pharmaceutically acceptable salts, and prodrugs are HSP90 inhibitors and are useful in the treating cellular proliferation diseases.

In one embodiment, the 2-amino-quinazolin-5-one compounds have formula (I):

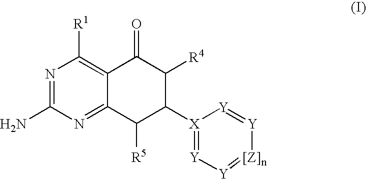

or a stereoisomer, tautomer, pharmaceutically acceptable salt, or prodrug thereof, wherein

n is 0 or 1;

wherein when n is 1, X is C, Y is at each position independently selected from CQ¹ and N, and Z is selected from CR² and N, and

wherein when n is 0, X is C or N, Y is at each position independently selected from CQ¹, N, NQ², O, and S;

wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;

wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;

wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkylamino,
(9) substituted or unsubstituted aryl,
(10) substituted or unsubstituted heteroaryl, and
(11) substituted or unsubstituted heterocyclyl;

wherein R² is selected from the group consisting of
(1) hydrogen,
(2) halogen,
(3) substituted or unsubstituted C₁-C₆ alkyl, and
(4) —OR³, —SR³, or —N(R³)₂;

wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;

wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino; and

with the proviso that when R¹ is methyl, and R⁴ and R⁵ are hydrogen, then X, Y, Z, and n together do not form an unsubstituted phenyl or furan-2-yl ring, and

with the proviso that when R¹, R⁴, and R⁵ are hydrogen, then X, Y, Z, and n together do not form a furan-2-yl, thien-2-yl, or phenyl ring wherein said ring is unsubstituted or substituted with one, two, or three substituents independently selected from the group consisting of C₁-C₆ alkyl, C₁-C₆ alkoxy, amino, alkylamino, dialkylamino, hydroxyl, and halo.

In another embodiment, 2-amino-quinazolin-5-one compounds have formula (Ia)

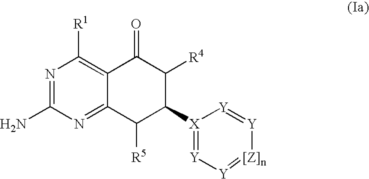

wherein R¹, R⁴, R⁵, X, Y, Z, and n are as defined for formula (I).

In another embodiment, 2-amino-quinazolin-5-one compounds have formula (II)

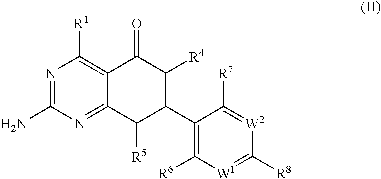

wherein W¹ and W² are independently N or CQ¹;

wherein R⁶ is selected from the group consisting of
(1) substituted or unsubstituted C₃-C₇ cycloalkyl,
(2) substituted or unsubstituted C₅-C₇ cycloalkenyl,
(3) substituted or unsubstituted aryl,
(4) substituted or unsubstituted heteroaryl, and
(5) substituted or unsubstituted heterocyclyl;

wherein R⁷ and R⁸ are independently
(1) hydrogen,
(2) halogen,
(3) substituted or unsubstituted C₁-C₆ alkyl,
(4) —OR³, —SR³, or —N(R³)₂, and

wherein Q¹, R¹, R³, R⁴, and R⁵ are as previously defined for formula (I).

In another embodiment, 2-amino-quinazolin-5-one compounds have formula (IIa)

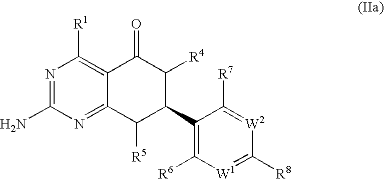

wherein R¹, R⁴, R⁵, R⁶, R⁷, R⁸, W¹, and W² are as previously defined for formula (I).

In some embodiments of the compounds of formula (II) or (IIa), W¹ is N. In some aspects, W² is N. In other aspects W¹ and W² are CQ¹. In some such aspects each Q¹ is hydrogen.

In some embodiments of the compounds of formula (II) or (IIa), R⁶ is selected from the group consisting of substituted aryl, substituted heterocyclyl, substituted heteroaryl, substituted C₃-C₇ cycloalkyl, and substituted C₅-C₇ cycloalkenyl, wherein said aryl, heterocyclyl, heteroaryl, C₃-C₇ cycloalkyl, and C₅-C₇ cycloalkenyl is selected from the group consisting of phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

In some embodiments of the compounds of formula (II) or (Ia), R⁶ is selected from the group consisting of (2-hydroxy-ethylamino)-pyrazin-2-yl, 1-methyl-1H-pyrazol-4-yl, 2-(5-methyl-pyridin-2-yl)-phenyl, 2,3-difluorophenyl, 2,4-difluorophenyl, 2,4-dimethoxyphenyl, 2,5-difluorophenyl, 2,6-difluorophenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-3-yl, 2-chloro-pyridin-4-yl, 2-difluoro-3-methoxyphenyl, 2-ethyl-phenyl, 2-fluoro-3-methoxy-phenyl, 2-fluoro-3-methylphenyl, 2-fluoro-4-methylphenyl, 2-fluoro-4-methyl-phenyl, 2-fluoro-5-methoxy-phenyl, 2-fluoro-5-methoxy-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-methoxy-5-trifluoromethyl-phenyl, 2-methoxyphenyl, 2-methoxy-pyridin-3-yl, 2-methoxy-pyrimidin-4-yl, 2-methylphenyl, 2-methyl-pyridin-3-yl, 2-oxo-1,2-dihydro-pyridin-3-yl, 2-phenoxyphenyl, 2-trifluoromethoxyphenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethoxy-phenyl, 3-trifluoromethylphenyl, 4,5-dimethoxy-pyrimidin-2-yl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-ethoxy-pyrimidin-5-yl, 4-ethyl-1H-pyrazol-3-yl, 4-fluorophenyl, 4-methoxy-5-methyl-pyrimidin-2-yl, 4-methoxy-5-methyl-pyrimidin-2-yl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-pyridin-2-yl, 4-methyl-pyridin-3-yl, 5,6-dimethoxy-pyrazin-2-yl, 5-acetyl-thiophen-2-yl, 5-amino-6-methoxy-3-methyl-pyrazin-2-yl, 5-amino-6-methoxy-pyrazin-2-yl, 5-chloro-4-methoxy-pyrimidin-2-yl, 5-chloro-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-trifluoromethyl-pyrimidin-2-yl, 6-acetyl-pyridin-2-yl, 6-chloro-pyrazin-2-yl, 6-ethoxy-pyrazin-2-yl, 6-ethyl-pyridin-2-yl, 6-fluoro-pyridin-2-yl, 6-fluoro-pyridin-3-yl, 6-hydroxy-pyridin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, and 6-trifluoromethyl-pyridin-2-yl.

In some embodiments of the compounds of formula (II) or (IIa), R⁷ is hydrogen.

In some embodiments of the compounds of formula (II) or (IIa), R⁸ is hydrogen, halo, or C₁-C₆ alkoxy. In some aspects, R⁸ is hydrogen. In other aspects R⁸ is fluoro. In still other aspects R⁸ is methoxy.

In another embodiment, the 2-amino-quinazolin-5-one compounds of the invention have formula (III):

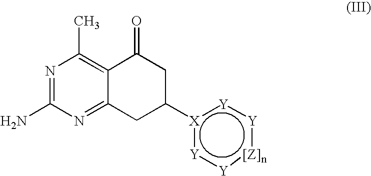

or a stereoisomer, tautomer, pharmaceutically acceptable salt, or prodrug thereof, wherein

n is 0 or 1,

wherein when n is 1, X is C, Y is at each position independently selected from CQ¹ and N, and Z is selected from CR² and N, and

wherein when n is 0, X is C or N, Y is at each position independently selected from CQ¹, N, NQ², O, and S;

wherein Q¹ is selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted and unsubstituted amino,
(12) —OR₃, —SR₃, or —N(R₃)₂,
(13) —C(O)R₃, —CO₂R₃, —C(O)N(R₃)₂, —S(O)R₃, —SO₂R₃, or —SO₂N(R₃)₂,
(14) —OC(O)R₃, —N(R₃)C(O)R₃, or —N(R₃)SO₂R₃
(15) —CN and
(16) —NO₂

wherein Q² is selected from the group consisting of
(1) hydrogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl, and
(10) substituted or unsubstituted heterocyclyl,

wherein R² is selected from the group consisting of
(1) hydrogen,
(2) halogen,
(3) substituted or unsubstituted C₁-C₃ alkyl, and
(4) halo-substituted or unsubstituted —OCH₃, —SCH₃, or —NHCH₃, and

wherein R³ is at each position independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted and unsubstituted amino,

with the proviso that when n is 1, —X is C, Y is CQ¹, and Z is CR², Q¹ and R² are not both hydrogen,

with the proviso that when n is 0, X is C, and Y adjacent to X is not O,

and with a further proviso that the total molecular weight does not exceed 750 Daltons.

In another embodiment, 2-amino-quinazolin-5-one compounds have formula (IV)

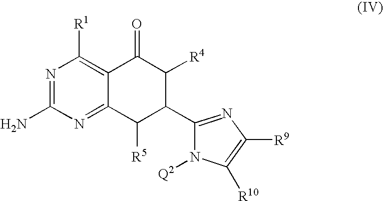

wherein R⁹ and R¹⁰ are independently Q¹, and R¹, R⁴, R⁵, Q¹, and Q² are as previously defined for formula (I).

In another embodiment, 2-amino-quinazolin-5-one compounds have formula (IVa)

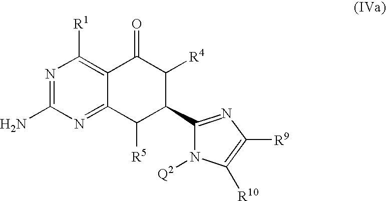

wherein R⁹ and R¹⁰ are independently Q¹, and R¹, R⁴, R⁵, Q¹, and Q² are as previously defined for formula (I).

In some aspects of the compounds of formula (IV) and (IVa), Q² is selected from the group consisting of substituted or unsubstituted aryl, substituted or unsubstituted heterocyclyl, substituted or unsubstituted heteroaryl, substituted or unsubstitued C₃-C₇ cycloalkyl, and substituted or unsubstitued C₅-C₇ cycloalkenyl. In other aspects said aryl, heterocyclyl, heteroaryl, C₃-C₇ cycloalkyl, and C₅-C₇ cycloalkenyl is selected from the group consisting of phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

In still other aspects Q² is selected from the group consisting of (2-hydroxy-ethylamino)-pyrazin-2-yl, 1-methyl-1H-pyrazol-4-yl, 2-(5-methyl-pyridin-2-yl)-phenyl, 2,3-difluorophenyl, 2,4-difluorophenyl, 2,4-dimethoxyphenyl, 2,5-difluorophenyl, 2,6-difluorophenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-3-yl, 2-chloro-pyridin-4-yl, 2-difluoro-3-methoxyphenyl, 2-ethyl-phenyl, 2-fluoro-3-methoxy-phenyl, 2-fluoro-3-methylphenyl, 2-fluoro-4-methylphenyl, 2-fluoro-4-methyl-phenyl, 2-fluoro-5-methoxy-phenyl, 2-fluoro-5-methoxy-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-methoxy-5-trifluoromethyl-phenyl, 2-methoxyphenyl, 2-methoxy-pyridin-3-yl, 2-methoxy-pyrimidin-4-yl, 2-methylphenyl, 2-methyl-pyridin-3-yl, 2-oxo-1,2-dihydro-pyridin-3-yl, 2-phenoxyphenyl, 2-trifluoromethoxyphenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethoxy-phenyl, 3-trifluoromethylphenyl, 4,5-dimethoxy-pyrimidin-2-yl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-ethoxy-pyrimidin-5-yl, 4-ethyl-1H-pyrazol-3-yl, 4-fluorophenyl, 4-methoxy-5-methyl-pyrimidin-2-yl, 4-methoxy-5-methyl-pyrimidin-2-yl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-pyridin-2-yl, 4-methyl-pyridin-3-yl, 5,6-dimethoxy-pyrazin-2-yl, 5-acetyl-thiophen-2-yl, 5-amino-6-methoxy-3-methyl-pyrazin-2-yl, 5-amino-6-methoxy-pyrazin-2-yl, 5-chloro-4-methoxy-pyrimidin-2-yl, 5-chloro-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-trifluoromethyl-pyrimidin-2-yl, 6-acetyl-pyridin-2-yl, 6-chloro-pyrazin-2-yl, 6-ethoxy-pyrazin-2-yl, 6-ethyl-pyridin-2-yl, 6-fluoro-pyridin-2-yl, 6-fluoro-pyridin-3-yl, 6-hydroxy-pyridin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, and 6-trifluoromethyl-pyridin-2-yl.

In one embodiment of the compounds of formula (IV) and (IVa), R⁹ and R¹⁰ are hydrogen. In another aspect one of R⁹ or R¹⁰ is hydrogen and the other is halo or C₁-C₆ alkoxy. In some aspects, one of R⁹ or R¹⁰ is fluoro. In other aspects one of R⁹ or R¹⁰ is methoxy.

In one embodiment, provided are pharmaceutical compositions comprising a pharmaceutically acceptable carrier and a compound having formula (V)

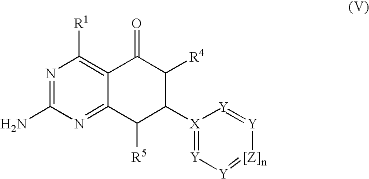

or a stereoisomer, tautomer, or pharmaceutically acceptable salt thereof, wherein

n is 0 or 1;

wherein when n is 1, X is C, Y is at each position independently selected from CQ¹ and N, and Z is selected from CR² and N, and

wherein when n is 0, X is C or N, Y is at each position independently selected from CQ¹, N, NQ², O, and S;

wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;

wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;

wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkylamino,
(9) substituted or unsubstituted aryl,
(10) substituted or unsubstituted heteroaryl, and
(11) substituted or unsubstituted heterocyclyl;

wherein R² is selected from the group consisting of
(1) hydrogen,
(2) halogen,
(3) substituted or unsubstituted C₁-C₆ alkyl, and
(4) —OR³, —SR³, or N

wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;

wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino.

For the compounds of formula (I), (Ia), (II), (IIa), (III), (IV), (IVa), and (V) representative substituted alkyl groups include arylalkyl, heteroarylalkyl, cycloalkylalkyl, heterocyclylalkyl, aminoalkyl, alkylaminoalkyl, dialkyaminoalkyl, and sulfonamidoalkyl groups.

Representative aryl groups include phenyl groups.

Representative heteroaryl groups include pyridyl, pyrazinyl, pyrimidinyl, pyridazinyl, pyrazolyl, indolyl, quinolinyl, oxazolyl, thiazolyl, and thienyl groups.

In one embodiment of the compounds of formula (I), (Ia), (II), (IIa), (IV), (IVa), and (V), R¹ is hydrogen or substituted or unsubstituted C₁-C₆ alkyl. In some aspects, R¹ is methyl.

In one embodiment of the compounds of formula (I), (Ia), (III), and (V), R² is hydrogen, halo, or C₁-C₆ alkoxy. In some aspects, R² is hydrogen. In other aspects R² is fluoro. In still other aspects R² is methoxy.

In one embodiment of the compounds of formula (I), (Ia), (II), (Ia), (IV), (IVa), and (V), one of R⁴ and R⁵ is hydrogen. In some aspects, both R⁴ and R⁵ are hydrogen.

In one embodiment of the compounds of formula (I), (Ia), (III), and (V), one of Q¹ or Q² is independently selected from substituted and unsubstituted phenyl, substituted and unsubstituted pyridyl, substituted and unsubstituted pyrimidinyl, substituted and unsubstituted pyrazinyl, substituted and unsubstituted indolyl, substituted and unsubstituted thiazolyl, and substituted and unsubstituted thienyl.

In another embodiment of the compounds of formula (I), (Ia), (III), and (V), one of Q¹ or Q² is independently selected from piperidinyl, morpholinyl, pyrrolidinonyl, and benzyl amino.

In another embodiment of the compounds of formula (I), (Ia), (III), and (V), one of Q¹ or Q² is independently selected from cyclohexyl and cyclopentyl.

In another embodiment of the compounds of formula (I), (Ia), (III), and (V), one of Q¹ or Q² is independently selected from cyclohexenyl and cyclopentenyl.

In another embodiment of the compounds of formula (I), (Ia), (III), and (V), and in combination of the any of the embodiments disclosed, one of Q¹, Q², R², or R³ is not hydrogen.

In some such aspects, at least one of Q¹, Q², R², or R³ is selected from the group consisting of substituted or unsubstituted aryl, substituted or unsubstituted heterocyclyl, substituted or unsubstituted heteroaryl, substituted or unsubstitued C₃-C₇ cycloalkyl, and substituted or unsubstitued C₅-C₇ cycloalkenyl. In other aspects said aryl, heterocyclyl, heteroaryl, C₃-C₇ cycloalkyl, and C₅-C₇ cycloalkenyl is selected from the group consisting of phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

In one embodiment of the compounds of formula (I), (Ia), (III), and (V), one of Q¹ or Q² is selected from the group consisting of (2-hydroxy-ethylamino)-pyrazin-2-yl, 1-methyl-1H-pyrazol-4-yl, 2-(5-methyl-pyridin-2-yl)-phenyl, 2,3-difluorophenyl, 2,4-difluorophenyl, 2,4-dimethoxyphenyl, 2,5-difluorophenyl, 2,6-difluorophenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-3-yl, 2-chloro-pyridin-4-yl, 2-difluoro-3-methoxyphenyl, 2-ethyl-phenyl, 2-fluoro-3-methoxy-phenyl, 2-fluoro-3-methylphenyl, 2-fluoro-4-methylphenyl, 2-fluoro-4-methyl-phenyl, 2-fluoro-5-methoxy-phenyl, 2-fluoro-5-methoxy-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-methoxy-5-trifluoromethyl-phenyl, 2-methoxyphenyl, 2-methoxy-pyridin-3-yl, 2-methoxy-pyrimidin-4-yl, 2-methylphenyl, 2-methyl-pyridin-3-yl, 2-oxo-1,2-dihydro-pyridin-3-yl, 2-phenoxyphenyl, 2-trifluoromethoxyphenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethoxy-phenyl, 3-trifluoromethylphenyl, 4,5-dimethoxy-pyrimidin-2-yl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-ethoxy-pyrimidin-5-yl, 4-ethyl-1H-pyrazol-3-yl, 4-fluorophenyl, 4-methoxy-5-methyl-pyrimidin-2-yl, 4-methoxy-5-methyl-pyrimidin-2-yl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-pyridin-2-yl, 4-methyl-pyridin-3-yl, 5,6-dimethoxy-pyrazin-2-yl, 5-acetyl-thiophen-2-yl, 5-amino-6-methoxy-3-methyl-pyrazin-2-yl, 5-amino-6-methoxy-pyrazin-2-yl, 5-chloro-4-methoxy-pyrimidin-2-yl, 5-chloro-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-trifluoromethyl-pyrimidin-2-yl, 6-acetyl-pyridin-2-yl, 6-chloro-pyrazin-2-yl, 6-ethoxy-pyrazin-2-yl, 6-ethyl-pyridin-2-yl, 6-fluoro-pyridin-2-yl, 6-fluoro-pyridin-3-yl, 6-hydroxy-pyridin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, and 6-trifluoromethyl-pyridin-2-yl.

In one embodiment of the compounds of formula (I), (Ia), (II), (Ia), (III), (IV), (IVa) and (V), R³ is selected from the group consisting of methyl, ethyl, isopropyl, cyclopentyl, and cyclohexyl.

In another embodiment, of the compounds of formula (I), (Ia), (II), (Ia), (III), (IV), (IVa), and (V), R³ is selected from substituted and unsubstituted phenyl, substituted and unsubstituted thiazolyl, substituted and unsubstituted pyridyl, substituted and unsubstituted pyrazinyl, and substituted and unsubstituted pyrimidinyl.

In another embodiment, of the compounds of formula (I), (Ia), (II), (IIa), (III), (IV), (IVa), and (V), R³ is selected from the group consisting of 2-aminoethyl, 2-piperidinylethyl, 2-piperazinylethyl, 2-morpholinylethyl, and 2-(N-methylpiperazinyl)ethyl.

In one embodiment, present invention provides a compound or a stereoisomer, tautomer, pharmaceutically acceptable salt, or prodrug thereof selected from the compounds in Tables I and II. In another embodiment, the invention provides a composition comprising a pharmaceutically acceptable carrier and a compound or a stereoisomer, tautomer, pharmaceutically acceptable salt, or prodrug thereof selected from the compounds in Tables I and II.

In another embodiment, the compounds of the present invention exhibit helical asymmetry. More particularly, the compounds of the present invention may be atropisomers, which is a subclass of conformers that can be isolated as separate chemical species and which arise from restricted rotation about a single bond.

In other aspects, the present invention provides methods for manufacture of 2-amino-quinazolin-5-one compounds. Methods of making representative compounds of the invention are described in Examples 1-19. It is further contemplated that, in addition to the compounds of formula (I), intermediates, and their corresponding methods of syntheses are included within the scope of the invention.

In other aspects, the present invention provides compositions that include the HSP90 inhibitors described herein, and methods that utilize the HSP90 inhibitors described herein.

In one aspect, the present invention provides pharmaceutical compositions comprising at least one 2-amino-quinazolin-5-one compound (e.g., a compound of formula (I), (Ia), (II), (IIa), (III), (IV), (IVa), and (V)) together with a pharmaceutically acceptable carrier suitable for administration to a human or animal subject, either alone or together with other anticancer agents.

A number of suitable anticancer agents to be used as combination therapeutics are contemplated for use in the compositions and methods of the present invention. Suitable anticancer agents to be used in combination with the compounds of the invention include agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a] and interleukins [e.g., IL-2]); adoptive immunotherapy agents; hematopoietic growth factors; agents that induce tumor cell differentiation (e.g., all-trans-retinoic acid); gene therapy reagents; antisense therapy reagents and nucleotides; tumor vaccines; inhibitors of angiogenesis, and the like. Numerous other examples of chemotherapeutic compounds and anticancer therapies suitable for co-administration with the 2-amino-quinazolin-5-one compounds of the invention are known to those skilled in the art.

In certain embodiments, anticancer agents to be used in combination with 2-amino-quinazolin-5-one compounds of the invention comprise agents that induce or stimulate apoptosis. Agents that induce apoptosis include, but are not limited to, radiation; kinase inhibitors (e.g., Epidermal Growth Factor Receptor [EGFR] kinase inhibitor, Vascular Endothelial Growth Factor Receptor [VEGFR] kinase inhibitor, Fibroblast Growth Factor Receptor [FGFR] kinase inhibitor, Platelet-derived Growth Factor Receptor [PGFR] I kinase inhibitor, and Bcr-Abl kinase inhibitors such as STI-571 [Gleevec or Glivec]); antisense molecules; antibodies [e.g., Herceptin and Rituxan]; anti-estrogens [e.g., raloxifene and tamoxifen]; anti-androgens [e.g., flutamide, bicalutamide, finasteride, amino-glutethamide, ketoconazole, and corticosteroids]; cyclooxygenase 2 (COX-2) inhibitors [e.g., Celecoxib, meloxicam, NS-398, and non-steroidal anti-inflammatory drugs (NSAIDs)]; and cancer chemotherapeutic drugs [e.g., irinotecan (Camptosar), CPT-11, fludarabine (Fludara), dacarbazine (DTIC), dexamethasone, mitoxantrone, Mylotarg, VP-16, cisplatinum, 5-FU, Doxrubicin, Taxotere or Taxol]; cellular signaling molecules; ceramides and cytokines; and staurosparine; and the like.

In other aspects, the invention provides methods for using the compounds and compositions described herein. For example, the compounds and compositions described herein can be used in the treatment of cancer. The compounds and compositions described herein can also be used in the manufacture of a medicament for the treatment of cancer.

In one embodiment, the present invention provides methods of treating human or animal subjects suffering from a cellular proliferative disease, such as cancer. The present invention provides methods of treating a human or animal subject in need of such treatment, comprising administering to the subject a therapeutically effective amount of an 2-amino-4-quinazolin-5-one compound or composition (e.g., a compound of formula (I), (Ia), (II), (IIa), (III), (IV), (IVa), or a composition of formula (V)), either alone or in combination with other anticancer agents.

In another embodiment, the present invention provides methods for treating a cellular proliferative disease in a human or animal subject in need of such treatment comprising, administering to said subject an amount of an 2-amino-quinazolin-5-one compound or composition (e.g., a compound of formula (I), (Ia), (II), (Ia), (III), (IV), (IVa), or a composition of formula (V)) effective to reduce or prevent cellular proliferation or tumor growth in the subject.

In another embodiment, the present invention provides methods for treating a cellular proliferative disease in a human or animal subject in need of such treatment comprising administering to said subject an amount of an 2-amino-quinazolin-5-one compound (e.g., a compound of formula (I), (Ia), (II), (IIa), (III), (IV), (IVa), or a composition of formula (V)) effective to reduce or prevent cellular proliferation in the subject in combination with at least one additional agent for the treatment of cancer.

The present invention provides compounds that are inhibitors of HSP90. The inhibitors are useful in pharmaceutical compositions for human or veterinary use where inhibition of HSP90 is indicated, e.g., in the treatment of cellular proliferative diseases such as tumor and/or cancerous cell growth mediated by HSP90. In particular, the compounds are useful in the treatment of human or animal (e.g., murine) cancers, including, for example, lung and bronchus; prostate; breast; pancreas; colon and rectum; thyroid; stomach; liver and intrahepatic bile duct; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-hodgkin lymphoma; melanoma; and villous colon adenoma.

In another embodiment, the invention provides methods of treating an HSP90 mediated disorder. In one method, an effective amount of an 2-amino-4-quinazolin-5-one compound is administered to a patient (e.g., a human or animal subject) in need thereof to mediate (or modulate) HSP90 activity.

A representative assay for determining HSP90 inhibitory activity is described in Example 21. In a preferred embodiment, the 2-amino-quinazolin-5-one compounds of the invention have an IC₅₀ value for inhibiting HSP90 activity less than or equal to 100 μM. In more preferred embodiments, the IC₅₀ value is less than or equal to 50 μM, even more preferred with an IC₅₀ value less than or equal to 25 μM. Still more preferred embodiment have IC₅₀ values less than or equal to 10 μM, and even more preferred embodiments have IC₅₀ values less than or equal to 1 μM.

The following definitions are provided to better understand the invention.

“Alkyl” or “unsubstituted alkyl” refers to hydrocarbyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)—CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂—CH(CH₃)₂, —CH(CH₃)CH(CH₃)CH(CH₃)₂, —CH(CH₂CH₃)CH(CH₃)CH(CH₃)(CH₂CH₃), and others. Thus the phrase “alkyl groups” includes primary alkyl groups, secondary alkyl groups, and tertiary alkyl groups. Preferred alkyl groups include straight and branched chain alkyl groups having 1 to 12, 1 to 6, or 1 to 3 carbon atoms.

“Alkylene” or “unsubstituted alkylene” refers to the same residues as noted above for “alkyl,” but having two points of attachment. Exemplary alkylene groups include ethylene (—CH₂CH₂—), propylene (—CH₂CH₂CH₂—), and dimethylpropylene (—CH₂C(CH₃)₂CH₂—).

“Alkenyl” or “unsubstitued alkenyl” refers to straight chain and branched hydrocarbyl radicals having one or more carbon-carbon double bonds and from 2 to about 20 carbon atoms. Preferred alkenyl groups include straight chain and branched alkenyl groups having 2 to 12, or 2 to 6 carbon atoms.

“Alkynyl” or “unsubstitued alkynyl” refers to straight chain and branched hydrocarbyl radicals having one or more carbon-carbon triple bonds and from 2 to about 20 carbon atoms. Preferred alkynyl groups include straight chain and branched alkynyl groups having 2 to 12, or 2 to 6 carbon atoms.

“Cycloalkyl” or “unsubstituted cycloalkyl” refers to a mono- or polycyclic alkyl substituent. Representative cycloalkyl groups include cyclopropyl, cyclobutyl, cyclopentyl, cyclohexyl, cycloheptyl, and cyclooctyl. Preferred cycloalkyl groups have 3 to 7 carbon atoms.

“Cycloalkenyl” or “unsubstitued cycloalkenyl” refers to a mono- or polycyclic alkyl substituents having at least one ring carbon-carbon double bond. Preferred cycloalkenyl groups have 5 to 7 carbon atoms and include cyclopentenyl and cyclohexenyl.

“Substituted alkyl” refers to an alkyl group as defined above in which one or more bonds to a carbon(s) or hydrogen(s) are replaced by a bond to non-hydrogen and non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines. Substituted alkyl groups also include groups in which one or more bonds to a carbon(s) or hydrogen(s) atom is replaced by a higher-order bond (e.g., a double- or triple-bond) to a heteroatom such as oxygen in oxo, carbonyl, carboxyl, and ester groups; nitrogen in groups such as imines, oximes, hydrazones, and nitriles. Substituted alkyl groups further include alkyl groups in which one or more bonds to a carbon(s) or hydrogen(s) atoms is replaced by a bond to an aryl, heteroaryl, heterocyclyl, cycloalkyl, or cycloalkenyl group. Preferred substituted alkyl groups include, among others, alkyl groups in which one or more bonds to a carbon or hydrogen atom is/are replaced by one or more bonds to fluoro, chloro, or bromo group. Another preferred substituted alkyl group is the trifluoromethyl group and other alkyl groups that contain the trifluoromethyl group. Other preferred substituted alkyl groups include those in which one or more bonds to a carbon or hydrogen atom is replaced by a bond to an oxygen atom such that the substituted alkyl group contains a hydroxyl, alkoxy, or aryloxy group. Other preferred substituted alkyl groups include alkyl groups that have an amine, or a substituted or unsubstituted alkylamine, dialkylamine, arylamine, (alkyl)(aryl)amine, diarylamine, heterocyclylamine, diheterocyclylamine, (alkyl)(heterocyclyl)amine, or (aryl)(heterocyclyl)amine group. Still other preferred substituted alkyl groups include those in which one or more bonds to a carbon(s) or hydrogen(s) atoms is replaced by a bond to an aryl, heteroaryl, heterocyclyl, cycloalkyl, or heterocyloalkenyl group. Examples of substituted alkyl are: —(CH₂)₃NH₂, —CH₂)₃NH(CH₃), —(CH₂)₃NH(CH₃)₂, —CH₂C(═CH₂)CH₂NH₂, —CH₂C(═O)CH₂NH₂, —CH₂S(═O)₂CH₃, —CH₂OCH₂NH₂, —CO₂H. Examples of substituents of substituted alkyl are: —CH₃, —C₂H₅, —CH₂OH, —OH, —OCH₃, —OC₂H₅, —OCF₃, —CF₃, —OC(═O)CH₃, —OC(═O)NH₂, —OC(═O)N(CH₃)₂, —CN, —NO₂, —C(═O)C—H₃, —CO₂H, —CO₂CH₃, —CONH₂, —NH₂, —N(CH₃)₂, —NHSO₂CH₃, —NHCOCH₃, —NHC(═O)OCH₃, —NHSO—₂CH₃, —SO₂CH₃, —SO₂NH₂, and halo.

“Substituted alkenyl” has the same meaning with respect to unsubstituted alkenyl groups that substituted alkyl groups has with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

“Substituted alkynyl” has the same meaning with respect to unsubstituted alkynyl groups that substituted alkyl groups has with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

“Substituted cycloalkyl” has the same meaning with respect to unsubstituted cycloalkyl groups that substituted alkyl groups has with respect to unsubstituted alkyl groups.

“Substituted cycloalkenyl” has the same meaning with respect to unsubstituted cycloalkenyl groups that substituted alkyl groups has with respect to unsubstituted alkyl groups.

“Aryl” or “unsubstituted aryl” refers to monocyclic and polycyclic aromatic groups that do not contain ring heteroatoms. Exemplary aryl moieties employed as substituents in compounds of the present invention include phenyl, naphthyl, and the like.

“Aralkyl” or “arylalkyl” refers to an alkyl group substituted with an aryl group as defined above. Typically, aralkyl groups employed in compounds of the present invention have from 1 to 6 carbon atoms incorporated within the alkyl portion of the aralkyl group. Suitable aralkyl groups employed in compounds of the present invention include, for example, benzyl and the like. “Heteroarylalkyl” or “heteroaralkyl” refers to an alkyl group substituted with a heteroaryl group as defined above. Typically, heteroarylalkyl groups employed in compounds of the present invention have from 1 to 6 carbon atoms incorporated within the alkyl portion of the aralkyl group. Suitable heteroarylalkyl groups employed in compounds of the present invention include, for example, picolyl and the like.

“Alkoxy” refers to RO— wherein R is C₁-C₇ alkyl. Representative examples of alkoxy groups include methoxy, ethoxy, t-butoxy, trifluoromethoxy, and the like.

“Amidino” refers to the moieties R—C(═N)—NR′ (the radical being at the “N¹” nitrogen) and R(NR′)C═N— (the radical being at the “N²” nitrogen), where R and R′ can be hydrogen, C₁-C₇ alkyl, C₅-C₇ aryl, or C₅-C₇ aralkyl.

“Amino” refers herein to the group —NH₂. The term “substituted amino” and “alkylamino” refers herein to the group —NRR′ where R is C₁-C₇ alkyl and R′ is hydrogen or C₁-C₇ alkyl. The term “dialkylamino” refers herein to the group —NRR′ where R and R′ are independently C₁-C₇ alkyl. The term “arylamino” refers herein to the group —NRR′ where R is C₅-C₇ aryl and R¹ is hydrogen, C₁-C₇ alkyl, or C₅-C₇ aryl. The term “aralkylamino” refers herein to the group —NRR′ where R is aralkyl and R′ is hydrogen, C₁-C₇ alkyl, C₅-C₇ aryl, or C₅-C₇ aralkyl. “Benzylamino” refers to the group —NHCH₂Ph.

“Aminoalkyl” refers to an alkyl group substituted with an amino group. “Alkylaminoalkyl” and “dialkylaminoalkyl” refers to an alkyl group substituted respectively with an alkylamino or dialkylamino group as defined above.

“Alkoxyalkyl” refers to the group -alk₁-O-alk₂ where alk₁ is C₁-C₇ alkyl and alk₂ is C₁-C₇ alkyl. The term “aryloxyalkyl” refers to the group —C₁-C₇ alkyl-O—C₅-C₇ aryl. “Alkoxyalkylamino” refers herein to the group —NR-(alkoxyalkyl), where R includes hydrogen, C₅-C₇ aralkyl, or C₁-C₇ alkyl.

“Aminocarbonyl” refers herein to the group —C(O)—NH₂. “Substituted aminocarbonyl” refers herein to the group —C(O)—NRR′ where R is C₁-C₇ alkyl and R¹ is hydrogen or C₁-C₇ alkyl. The term “arylaminocarbonyl” refers herein to the group —C(O)—NRR′ where R is C₅-C₇ aryl and R¹ is hydrogen, C₁-C₇ alkyl or C₅-C₇ aryl. “Aralkylaminocarbonyl” refers herein to the group —C(O)—NRR′ where R is C₅-C₇ aralkyl and R¹ is hydrogen, C₁-C₇ alkyl, C₅-C₇ aryl, or C₅-C₇ aralkyl.

“Aminosulfonyl” refers herein to the group —S(O)₂—NH₂. “Substituted aminosulfonyl” refers herein to the group —S(O)₂—NRR′ where R is C₁-C₇ alkyl and R¹ is hydrogen or C₁-C₇ alkyl. The term “aralkylaminosulfonlyaryl” refers herein to the group —C₅-C₇ aryl-S(O)₂—NH-aralkyl.

“Aryloxy” refers to RO— wherein R is aryl.

“Carbonyl” refers to the divalent group —C(O)—. “Alkylcarbonyl” refers to the group —C(O)alkyl. “Arylcarbonyl” refers to the group —C(O)aryl. Similarly, the term “heteroarylcarbonyl”, “aralkylcarbonyl”, and “heteroaralkylcarbonyl” refers to —C(O)—R where R is respectively heteroaryl, aralkyl, and heteroaralkyl.

“Carbonyloxy”-refers generally to the group —C(O)—O. Such groups include esters, —C(O)—O—R, where R is C₁-C₇ alkyl, C₃-C₇ cycloalkyl, C₅-C₇ aryl, or C₅-C₇ aralkyl. The term “arylcarbonyloxy” refers herein to the group —C(O)—O—(C₅-C₇ aryl). The term “aralkylcarbonyloxy” refers herein to the group —C(O)—O—(C₅-C₇ aralkyl).

“Cycloalkylalkyl” refers to an alkyl group substituted with a cyloalkyl group as defined above. Typically, cycloalkylalkyl groups have from 1 to 6 carbon atoms incorporated within the alkyl portion of the cycloalkylalkyl group.

“Carbonylamino” refers to the divalent group —NH—C(O)— in which the hydrogen atom of the amide nitrogen of the carbonylamino group can be replaced C₁-C₇ alkyl, C₅-C₇ aryl, or C₅-C₇ aralkyl group. Such groups include moieties such as carbamate esters (—NH—C(O)—O—R) and amides —NH—C(O)—R, where R is a straight or branched chain C₁-C₇ alkyl, C₃-C₇ cycloalkyl, or C₅-C₇ aryl or C₅-C₇ aralkyl. The term “alkylcarbonylamino” refers to —NH—C(O)—R where R is alkyl having from 1 to about 7 carbon atoms in its backbone structure. The term “arylcarbonylamino” refers to group —NH—C(O)—R where R is an C₅-C₇ aryl. Similarly, the term “aralkylcarbonylamino” refers to —NH—C(O)—R where R is C₅-C₇ aralkyl.

“Guanidino” or “guanidyl” refers to moieties derived from guanidine, H₂N—C(═NH)—NH₂. Such moieties include those bonded at the nitrogen atom carrying the formal double bond (the “2”-position of the guanidine, e.g., diaminomethyleneamino, (H₂N)₂C═NH— and those bonded at either of the nitrogen atoms carrying a formal single bond (the “1-” and/or “3”-positions of the guandine, e.g., H₂N—C(═NH)—NH—. The hydrogen atoms at any of the nitrogens can be replaced with a suitable substituent, such as C₁-C₇ alkyl, C₅-C₇ aryl, or C₅-C₇ aralkyl.

“Halogen” or “halo” refers to chloro, bromo, fluoro, and iodo groups. The term “haloalkyl” refers to an alkyl radical substituted with one or more halogen atoms. The term “haloalkoxy” refers to an alkoxy radical substituted with one or more halogen atoms.

“Hydroxyl” or “hydroxyl” refers to the group —OH.

“Heterocyclic” or “unsubstituted heterocyclic group,” “heterocycle” or “unsubstituted heterocycle,” and “heterocyclyl” or “unsubstituted heterocyclyl,” as used herein refers to any aromatic or non-aromatic monocyclic or polycyclic ring compounds containing a heteroatom selected from nitrogen, oxygen, or sulfur. Examples include 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-2 double bonds and the 6-membered ring has 0-3 double bonds; wherein the nitrogen and sulfur atom maybe optionally oxidized; wherein the nitrogen and sulfur heteroatoms maybe optionally quarternized; and including any bicyclic group in which any of the above heterocyclic rings is fused to a benzene ring or another 5- or 6-membered heterocyclic ring independently defined above. The term “heterocycle” thus includes rings in which nitrogen is the heteroatom as well as partially and fully-saturated rings and also includes fused and non-fused cyclic structures in which at least one cyclic structure is aromatic, such as, for example, benzodioxozolo (which has a heterocyclic structure fused to a phenyl group, i.e., 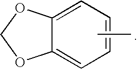
Preferred heterocycles have 3 to 14 ring atoms and include, for example: diazapinyl, pyrroyl, pyrrolidinyl, pyrazolyl, pyrazolidinyl, imidazoyl, imidazolidinyl, pyridyl, piperidinyl, pyrazinyl, piperazinyl, azetidinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl, quinoxalinyl, phthalazinyl, naphthpyridinyl, indazolyl, and benzothienyl.

Heterocyclic moieties can be, for example, monosubstituted or disubstituted with various substituents independently selected from but not limited to hydroxy, alkoxy, halo, oxo (C═O), alkylimino (RN═, wherein R is alkyl or alkoxy group), amino, alkylamino, dialkylamino, acylaminoalkyl, alkoxy, thioalkoxy, polyalkoxy, alkyl, cycloalkyl or haloalkyl.

The heterocyclic groups may be attached at various positions as shown below as will be apparent to those having skill in the organic and medicinal chemistry arts in conjunction with the disclosure herein 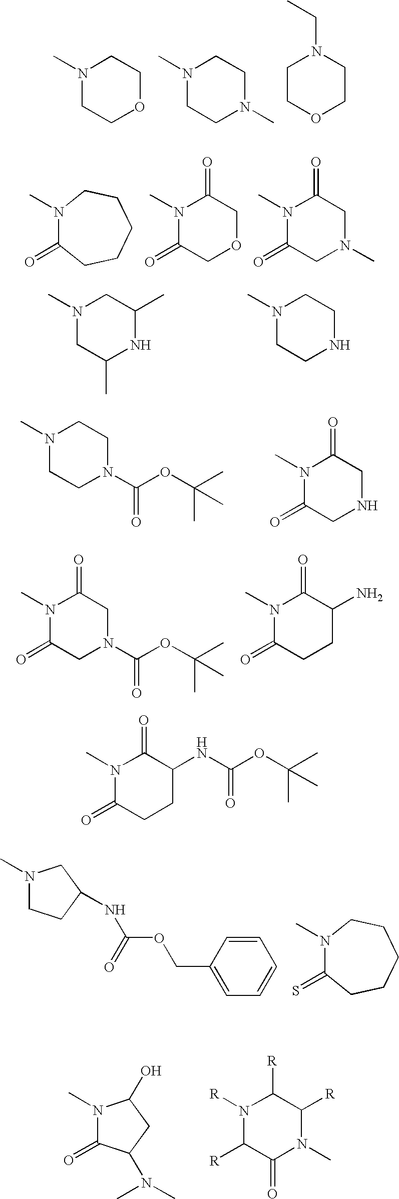
where R is H or a heterocyclic substituent, as described herein.

“Heteroaryl” or “unsubstituted heteroaryl” refers herein to an aromatic heterocyclyl group having from 1 to 4 heteroatoms as ring atoms in an aromatic ring with the remainder of the ring atoms being carbon atoms. Preferred heteroaryl groups have 5 to 14 ring atoms. Representative heteroaryls include, for example, imidazolyl, pyridyl, pyrazinyl, pyrimidinyl, pyridazinyl, pyrazolyl, indolyl, quinolinyl, oxazolyl, thienyl, thiazolyl, triazolyl, benzimidazolyl, benzothiazolyl, and benzoxazolyl. Heteroaryl groups can be further substituted and may be attached at various positions as will be apparent to those having skill in the organic and medicinal chemistry arts in conjunction with the disclosure herein. Representative substituted and unsubstituted heteroaryl groups include, for example, those found in the compounds disclosed in this application and in the examples shown below

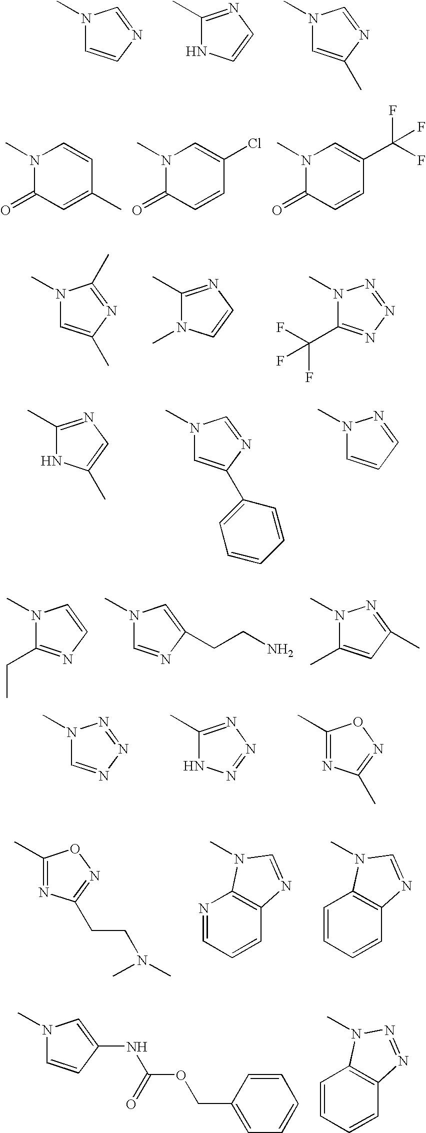

“Heteroarylalkyl” or “heteroaralkyl” refers to an alkyl group substituted with a heteroaryl group as defined above. Typically, heteroarylalkyl groups have from 1 to 6 carbon atoms incorporated within the alkyl portion of the heteroarylalkyl group.

“Imino” refers to the group ═NH.

“Nitro” refers to the group NO₂.

“Sulfonyl” refers herein to the group —SO₂—. “Alkylsulfonyl” refers to a substituted sulfonyl of the structure —SO₂R— in which R is C₁-C₇ alkyl. Alkylsulfonyl groups employed in compounds of the present invention are typically alkylsulfonyl groups having from 1 to 6 carbon atoms in its backbone structure. Thus, typical alkylsulfonyl groups employed in compounds of the present invention include, for example, methylsulfonyl (i.e., where R is methyl), ethylsulfonyl (i.e., where R is ethyl), propylsulfonyl (i.e., where R is propyl), and the like. The term “arylsulfonyl” refers herein to the group —SO₂-aryl. The term “heterocyclylsulfonyl” refers herein to the group —SO₂-heterocyclyl. The term “aralkylsulfonyl” refers herein to the group —SO₂-aralkyl. The term “sulfonamido” refers herein to —SO₂NH₂. The term “sulfonamidoalkyl” refers to (alkyl)SO₂NH₂—.

“Thio” or “thiol” refers to the group —SH. “Alkylthio” or “alkylthiol” refers to a thio group substituted with an alkyl group such as, for example, a C₁-C₆ alkyl group.

“Thioamido” refers to the group —C(═S)NH₂.

“Optionally substituted” refers to the optional replacement of hydrogen with a monovalent or divalent radical. “Substituted” refers to the replacement of hydrogen with a monovalent or divalent radical. Unless indicated otherwise, suitable substitution groups include, for example, hydroxyl, alkoxy, nitro, amino, imino, cyano, halo, thio, sulfonyl, thioamido, amidino, oxo, oxamidino, methoxamidino, guanidino, sulfonamido, carboxyl, formyl, alkyl, haloalkyl, alkylamino, haloalkylamino, alkoxy, haloalkoxy, alkoxyalkyl, alkylcarbonyl, aminocarbonyl, arylcarbonyl, aralkylcarbonyl, heteroarylcarbonyl, heteroaralkylcarbonyl, alkylthio, aminoalkyl, cyanoalkyl, aryl, and the like. Other suitable substitution groups include those substituents indicated for substituted alkyl. Examples of various suitable substitution groups are also found in reference to the compounds disclosed throughout this application.

The substitution group can itself be substituted. The group substituted onto the substitution group can be carboxyl, halo, nitro, amino, cyano, hydroxyl, alkyl, alkoxy, aminocarbonyl, —SR, thioamido, —SO₃H, —SO₂R, or cycloalkyl, where R is typically hydrogen, hydroxyl or alkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

Unless indicated otherwise, the nomenclature of substituents that are not explicitly defined herein are arrived at by naming the terminal portion of the functionality followed by the adjacent functionality toward the point of attachment. For example, the substituent “alkoxyheteroaryl” refers to the group (alkoxy)-(heteroaryl)-.

Preferred compounds of the invention have a total molecular weight less than 1000 Daltons, preferably less than 750 Daltons. Compounds of the invention typically have a minimum molecular weight of at least 150 Daltons. Preferred embodiments of the invention have a molecular weight between 150 and 750 Daltons, more preferred embodiments have a molecular weight between 200 and 500 Daltons. Other embodiments of the invention are compounds with a molecular weight between 300 and 450 Daltons. In another aspect of the invention compounds of the invention have a molecular weight between 350 and 400 Daltons.

Similarly, it is understood that the above definitions are not intended to include impermissible substitution patterns (e.g., methyl substituted with 5 fluoro groups). Such impermissible substitution patterns are well known to the skilled artisan.

“Carboxy-protecting group” refers to a carbonyl group which has been esterified with one of the commonly used carboxylic acid protecting ester groups employed to block or protect the carboxylic acid function while reactions involving other functional sites of the compound are carried out. In addition, a carboxy protecting group can be attached to a solid support whereby the compound remains connected to the solid support as the carboxylate until cleaved by hydrolytic methods to release the corresponding free acid. Representative carboxy-protecting groups include, for example, alkyl esters, secondary amides and the like.

Certain of the compounds of the invention comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of enantiomers, as well as enantiomers of the compounds of the invention are included in the present invention. The terms “S” and “R” configuration, as used herein, are as defined by the IUPAC 1974 “RECOMMENDATIONS FOR SECTION E, FUNDAMENTAL STEREOCHEMISTRY,” Pure Appl. Chem. 45:13-30, 1976. The terms α and β are employed for ring positions of cyclic compounds. The a-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the “Chemical Abstracts Index Guide,” Appendix IV, paragraph 203, 1987.

As used herein, the term “pharmaceutically acceptable salts” refers to the nontoxic acid or alkaline earth metal salts of the 2-amino-quinazolin-5-one compounds of the invention. These salts can be prepared in situ during the final isolation and purification of the 2-amino-quinazolin-5-one compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the 2-amino-quinazolin-5-one compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, and the like.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in Higuchi, T., and V. Stella, “Pro-drugs as Novel Delivery Systems,” A.C.S. Symposium Series 14, and in “Bioreversible Carriers in Drug Design,” in Edward B. Roche (ed.), American Pharmaceutical Association, Pergamon Press, 1987, both of which are incorporated herein by reference.

The term “HSP90 mediated disorder” refers to a disorder that can be beneficially treated by the inhibition of HSP90.

The term “cellular proliferative diseases” refers to diseases including, for example, cancer, tumor, hyperplasia, restenosis, cardiac hypertrophy, immune disorder and inflammation.

The term “cancer” refers to cancer diseases that can be beneficially treated by the inhibition of HSP90, including, for example, lung and bronchus; prostate; breast; pancreas; colon and rectum; thyroid; stomach; liver and intrahepatic bile duct; kidney and renal pelvis; urinary bladder; uterine corpus; uterine cervix; ovary; multiple myeloma; esophagus; acute myelogenous leukemia; chronic myelognous leukemia; lymphocytic leukemia; myeloid leukemia; brain; oral cavity and pharynx; larynx; small intestine; non-hodgkin lymphoma; melanoma; and villous colon adenoma.

The compounds of the invention are useful in vitro or in vivo in inhibiting the growth of cancer cells. The compounds may be used alone or in compositions together with a pharmaceutically acceptable carrier or excipient. Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-p-cyclodextrin, polyvinyl-pyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey, 1991, incorporated herein by reference.

Effective amounts of the compounds of the invention generally include any amount sufficient to detectably inhibit HSP90 activity by any of the assays described herein, by other HSP90 activity assays known to those having ordinary skill in the art, or by detecting an inhibition or alleviation of symptoms of cancer.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

The compounds of the present invention may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

While the compounds of the invention can be administered as the sole active pharmaceutical agent, they can also be used in combination with one or more other agents used in the treatment of cancer. Representative agents useful in combination with the compounds of the invention for the treatment of cancer include, for example, irinotecan, topotecan, gemcitabine, gefitinib, vatalanib, sunitinib, sorafenib, erlotinib, dexrazoxane, gleevec, herceptin, 5-fluorouracil, leucovorin, carboplatin, cisplatin, taxanes, tezacitabine, cyclophosphamide, vinca alkaloids, imatinib, anthracyclines, rituximab, trastuzumab, topoisomerase I inhibitors, as well as other cancer chemotherapeutic agents.

The above compounds to be employed in combination with the compounds of the invention will be used in therapeutic amounts as indicated in the Physicians' Desk Reference (PDR) 47th Edition (1993), which is incorporated herein by reference, or such therapeutically useful amounts as would be known to one of ordinary skill in the art.

The compounds of the invention and the other anticancer agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

Antiestrogens, such as tamoxifen, inhibit breast cancer growth through induction of cell cycle arrest, that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to antiestrogen resistance (Donovan, et al, J. Biol. Chem. 276:40888, 2001). As reported by Donovan et al., inhibition of MAPK signaling through treatment with MEK inhibitor changed the phosphorylation status of p27 in hormone refactory breast cancer cell lines and in so doing restored hormone sensitivity. Accordingly, in one aspect, the compounds of formula (I), (Ia), (II), (IIa), (III), (IV), (IVa), or a composition of formula (V), may be used in the treatment of hormone dependent cancers, such as breast and prostate cancers, to reverse hormone resistance commonly seen in these cancers with conventional anticancer agents.

In hematological cancers, such as chronic myelogenous leukemia (CML), chromosomal translocation is responsible for the constitutively activated BCR-ABL tyrosine kinase. The afflicted patients are responsive to gleevec, a small molecule tyrosine kinase inhibitor, as a result of inhibition of Abl kinase activity. However, many patients with advanced stage disease respond to gleevec initially, but then relapse later due to resistance-conferring mutations in the Abl kinase domain. In vitro studies have demonstrated that BCR-Av1 employs the Raf kinase pathway to elicit its effects. In addition, inhibiting more than one kinase in the same pathway provides additional protection against resistance-conferring mutations. Accordingly, in another aspect of the invention, the compounds of formula (I), (Ia), (II), (IIa), (III), (IV), (IVa), or a composition of formula (V) are used in combination with at least one additional agent, such as gleevec, in the treatment of hematological cancers, such as chronic myelogenous leukemia (CML), to reverse or prevent resistance to the at least one additional agent.

In another aspect of the invention, kits that include one or more compounds of the invention are provided. Representative kits include a 2-amino-quinazolin-5-one compound of the invention (e.g., a compound of formula (I), (Ia), (II), (Ia), (III), (IV), (IVa), or a composition of formula (V)) and a package insert or other labeling including directions for treating a cellular proliferative disease by administering an HSP90 inhibitory amount of the compound.

In another embodiment, provided is a method of synthesizing a compound of formula (I), the comprising the steps of:
a) condensing a benzaldehyde compound with acetone to form a 4-phenylbut-3-en-2-one compound;
b) reacting said 4-phenylbut-3-en-2-one compound with a malonate ester and effecting decarboalkoxylation and dehydrative closure of that adduct to form a 5-phenyl-3-hydroxycyclohex-2-enone compound or a tautomer thereof;
c) acylating said 5-phenyl-3-hydroxycyclohex-2-enone compound or a tautomer thereof with an electrophilic acyl group to form a 3-oxo-5-phenylcyclohex-1-enyl ester compound;
d) rearranging said 3-oxo-5-phenylcyclohex-1-enyl ester compound with a catalytic nucleophile to form a 2-acyl-5-phenylcyclohexane-1,3-dione compound; and
e) condensing said 2-acyl-5-phenylcyclohexane-1,3-dione compound with guanidine to form a 2-amino-quinazolinone compound.

Schemes 1 and 2 below illustrates a general method for the preparation of intermediates and compounds of the embodiments. These compounds are prepared from starting materials either known in the art or commercially available. For illustrative purposes only, in Scheme 1, the X-Y-Z ring is bromophenyl.

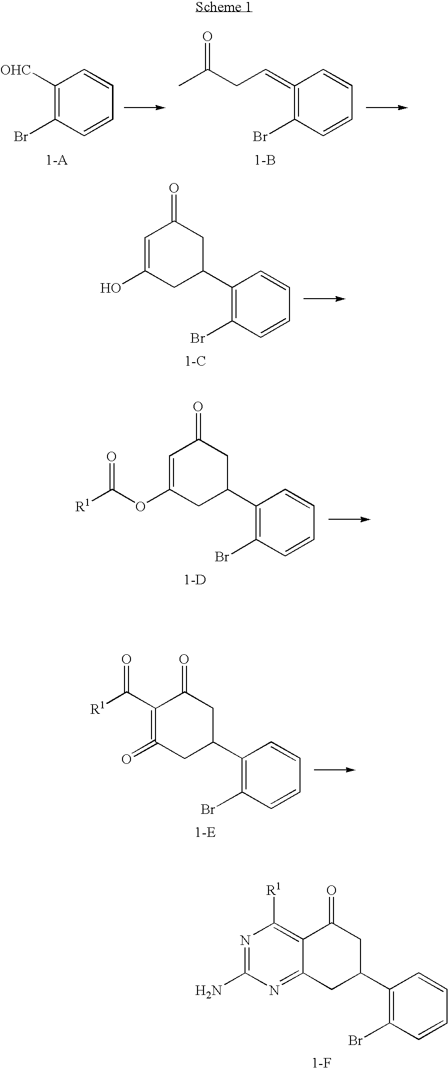

In one aspect, certain compounds of the embodiments can be prepared as shown in Scheme 1. 4-(2-Bromophenyl)but-3-en-2-one 1-B is prepared from bromobenzaldehyde 1-A with homologation with acetone. Cyclization after addition of methyl acetoacetate of 4-(2-bromophenyl)but-3-en-2-one 1-B gives 5-(2-bromophenyl)-3-hydroxycyclohex-2-enone 1-C. Reaction of 1-C with an acylating agent such as R¹COX (where X is a leaving group) in the presence of base gives ester 1-D. In the presence of a nucleophile, the acyl group rearranges to give dione 1-E. Subsequent reaction with guanidine gives 2-amino-quinzolinone 1-F.

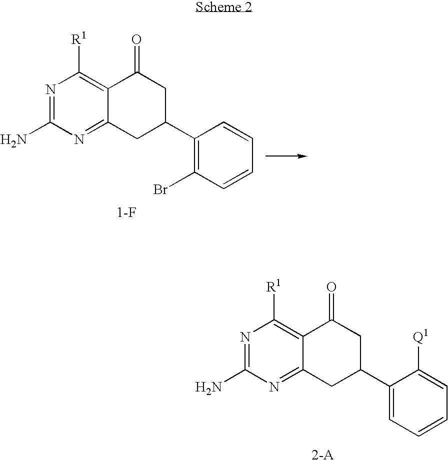

In one aspect, certain compounds of the embodiments can be prepared as shown in Scheme 2. Various compounds 2-A are prepared from 2-amino-7-(2-bromophenyl)-quinzolinone 1-F. For example in one instance, coupling of 1-F with an appropriate organotin derivative occurs in the presence of a palladium catalyst. In another instance, coupling of 1-F with an aryl derivative occurs via a Suzuki coupling using a boron ester or boronic acid derivative. In another instance, coupling of 1-F with an alcohol to form an ether occurs in the presence of cesium carbonate. In another instance, coupling of 1-F with an amine occurs in the presence of a base or other catalyst. In another instance, acylation of 1-F occurs with reaction of said compound with carbon monoxide and an alcohol. In another instance, amidation of 1-F can occur with reaction with formamide.

The present invention will be understood more readily by reference to the following examples, which are provided by way of illustration and are not intended to be limiting of the present invention.

### EXAMPLES

Referring to the examples that follow, compounds of the present invention were synthesized using the methods described herein, or other methods, which are well known in the art.

The compounds and/or intermediates were characterized by high performance liquid chromatography (HPLC) using a Waters Millenium chromatography system with a 2690 Separation Module (Milford, Mass.). The analytical columns were Alltima C-18 reversed phase, 4.6×250 mm from Alltech (Deerfield, Ill.). A gradient elution was used, typically starting with 5% acetonitrile/95% water and progressing to 100% acetonitrile over a period of 40 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.). In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; solvent system: 5-95% (or 35-95%, or 65-95% or 95-95%) acetonitrile in water with 0.05% TFA; flow rate 0.8 mL/min; molecular weight range 500-1500; cone Voltage 20 V; column temperature 40° C.) or a Hewlett Packard System (Series 1100 HPLC; Column: Eclipse XDB-C18, 2.1×50 mm; solvent system: 1-95% acetonitrile in water with 0.05% TFA; flow rate 0.4 mL/min; molecular weight range 150-850; cone Voltage 50 V; column temperature 30° C.). All masses were reported as those of the protonated parent ions.

GCMS analysis is performed on a Hewlett Packard instrument (HP6890 Series gas chromatograph with a Mass Selective Detector 5973; injector volume: 1 mL; initial column temperature: 50° C.; final column temperature: 259° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.). The spectral reference was either TMS or the known chemical shift of the solvent. Some compound samples were run at elevated temperatures (e.g., 75° C.) to promote increased sample solubility.

The purity of some of the invention compounds is assessed by elemental analysis (Desert Analytics, Tucson, Ariz.)

Melting points are determined on a Laboratory Devices Mel-Temp apparatus (Holliston, Mass.).

Preparative separations were carried out using a Flash 40 chromatography system and KP-Sil, 60A (Biotage, Charlottesville, Va.), or by flash column chromatography using silica gel (230-400 mesh) packing material, or by HPLC using a C-18 reversed phase column. Typical solvents employed for the Flash 40 Biotage system and flash column chromatography were dichloromethane, methanol, ethyl acetate, hexane, acetone, aqueous hydroxyamine, and triethyl amine. Typical solvents employed for the reverse phase HPLC were varying concentrations of acetonitrile and water with 0.1% trifluoroacetic acid.

The following are abbreviations used in the examples:
AcOH: Acetic acid
aq: Aqueous
ATP: Adenosine triphosphate
9-BBN 9-Borabicyclo[3.3.1]nonane
Boc: tert-Butoxycarbonyl
Celite Diatomaceous earth
DAP or Dap: Diaminopropionate
DCM: Dichloromethane
DEAD: Diethyl azodicarboxylate
DIEA: Diisopropylethylamine
DMA N,N-Dimethylacetamide
DMAP 4-Dimethylaminopyridine
DME: 1,2-Dimethoxyethane
DMF: N,N-Dimethylformamide
DMSO: Dimethyl sulfoxide
DPPA: Diphenyl phosphoryl azide
Et₃N: Triethylamine
EDC: N-(3-Dimethylaminopropyl)-N′-ethylcarbodiimide
EDCI: 1-(3-Dimethylaminopropyl)-3-ethylcarbodiimide
EtOAc: Ethyl acetate
EtOH: Ethanol
Fmoc: 9-Fluorenylmethoxycarbonyl
GC Gas Chromatography
Gly-OH: Glycine
HATU: O-(7-Azabenzotriaazol-1-yl)-N,N,N ‘N’-tetramethyluronium hexafluorophosphate
HBTU: 2-(1H-Benzotriazol-1-yl)-1,1,3,3-tetramethyluronium hexafluorophosphate
Hex: Hexane
HOAT 1-Hydroxy-7-azabenzotriazole
HOBT: 1-Hydroxybenzotriazole
HPLC: High performance liquid chromatography
NIS N-Iodosuccinimide
IC₅₀ value: The concentration of an inhibitor that causes a 50% reduction in a measured activity.
iPrOH: Isopropanol
LC/MS: Liquid chromatography/mass spectrometry
LRMS: Low resolution mass spectrometry
MeOH: Methanol
NaOMe: Sodium methoxide
nm: Nanometer
NMP: N-Methylpyrrolidone
PPA Polyphosphoric acid
PPh₃: Triphenyl phosphine
PTFE Polytetrafluoroethylene
PyBOP Benzotriazole-1-yl-oxy-tris-pyrrolidino-phosphonium
RP-HPLC: Reversed-phase high-performance liquid chromatography
RT: Room temperature
sat: Saturated
TEA: Triethylamine
TFA: Trifluoroacetic acid
THF: Tetrahydrofuran
TMS: Trimethylsilane
Thr: Threonine
TLC: Thin layer chromatography
Trt-Br: Triphenylmethyl bromide

Nomenclature for the compounds disclosed in this application was provided using ACD Name version 5.07 software (Nov. 14, 2001), ACD Name Batch version 5.04 (May 28, 2002) available from Advanced Chemistry Development, Inc., or by using AutoNom 2000 (Automatic Nomenclature) for ISIS/Base, implementing IUPAC standardized nomenclature. Other compounds, intermediates, and starting materials were named using standard IUPAC nomenclature.

It should be understood that the organic compounds according to the invention may exhibit the phenomenon of tautomerism. As the chemical structures within this specification can only represent one of the possible tautomeric forms, it should be understood that the invention encompasses any tautomeric form of the drawn structure.

It is understood that the invention is not limited to the embodiments set forth herein for illustration, but embraces all such forms thereof as come within the scope of the above disclosure.

The following examples illustrate methods for making representative compounds of the invention.

### Example 1

Representative Method for Synthesizing 2-Amino-quinazolin-5-one Compounds: Method A

In this example, a method for making representative compounds of the invention (Method A) is described.

Step 1:

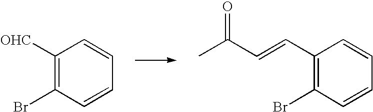

(E)-4-(2-Bromophenyl)but-3-en-2-one: Combined 5.00 g (0.027 mol) of 2-bromobenzaldehyde, 4.32 g (0.0743 mol) of acetone, and 25 mL of water in a 100 mL round bottom flask fitted with magnetic stirrer. The mixture was heated to 65° C., then 6.5 mL (0.00165 mol) of 1% aqueous sodium hydroxide was added at once. The reaction was stirred at 65° C. for an additional 1.5 h, then cooled to room temperature and neutralized to pH 6 with conc. aqueous hydrochloric acid. The reaction mixture was partitioned with ethyl acetate. The aqueous layer was extracted with ethyl acetate and the combined organics were dried over magnesium sulfate, filtered and concentrated under reduced pressure affording 5.86 g (96% yield) of the title compound as a yellow oil.

Step 2:

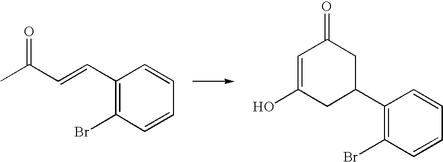

5-(2-Bromophenyl)-3-hydroxycyclohex-2-enone: Dissolved 0.66 g (0.029 mol) of sodium in 25 mL of anhydrous methanol. After formation of sodium methoxide was complete, added 3.78 g (0.0286 mol) methyl acetoacetate dropwise over 20 min. The reaction mixture was then heated to 50° C. and (E)-4-(2-bromophenyl)but-3-en-2-one in 10 mL of methanol was added dropwise over 30 min. The reaction mixture was heated for an additional hour at reflux, then quenched with 25 mL of water. Methanol was removed, 9.5 mL of 6 M aqueous sodium hydroxide was added and the mixture was heated at 80° C. for 1 h. After cooling to room temperature, the aqueous mixture was washed with 50 mL of toluene. The aqueous layer was heated to 100° C. and 9.5 mL of conc. aqueous hydrochloric acid was added dropwise over 30 min with vigorous gas evolution. The mixture was stirred for an additional 1 h at reflux, then cooled to room temperature. The solids were collected by filtration, washed with water, and dried under vacuum. Trituration with 20 mL of ether afforded 5.79 g (83% yield) of the title compound as a white solid.

Step 3:

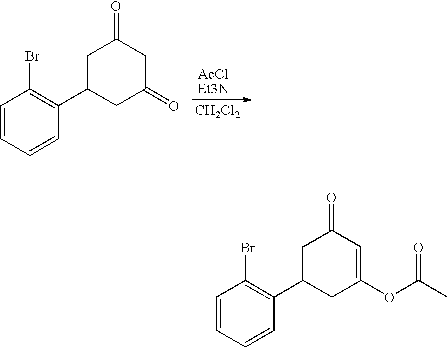

5-(2-Bromophenyl)-3-oxocyclohex-1-enyl acetate: Combined 9.89 g (0.037 mol) of compound prepared in step 2 with 180 mL of dichloromethane. The solution was cooled to 0° C., charged with 5.7 mL (4.1 g, 0.41 mol) of triethylamine followed by dropwise addition over 20 min of 2.9 mL (3.2 g, 0.041 mol) of acetyl chloride. After stirring for 30 min at 0° C., the reaction mixture was allowed to warm to room temperature, then quenched with 200 mL of water. The organic phase was collected and dried over magnesium sulfate, filtered, and concentrated in vacuo to afford 11.11 g (96% yield) of the title compound as a clear, orange oil.

Step 4:

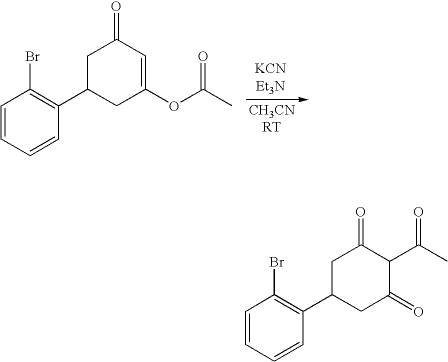

2-Acetyl-5-(2-bromophenyl)cyclohexane-1,3-dione: Combined 11.11 g (0.037 mol) of the compound prepared in step 3 with 100 mL of acetonitrile, 5.7 mL (0.041 mmol) of triethyl amine and 0.48 g (0.20 mol) of potassium cyanide. The reaction mixture was stirred for 16 h at room temperature. The acetonitrile was removed under reduced pressure and the resulting residue was taken up in 200 mL of ethyl acetate. The resulting solution was washed with 200 mL of 1 N aqueous HCl followed by 200 mL of water. The organic layer was separated, dried over magnesium sulfate, filtered and concentrated in vacuo to afford 10.74 g (97% yield) of the title compound as a pale yellow solid that could be further purified by silica gel chromatography eluting with 4:1 hexane/ethyl acetate.

Step 5:

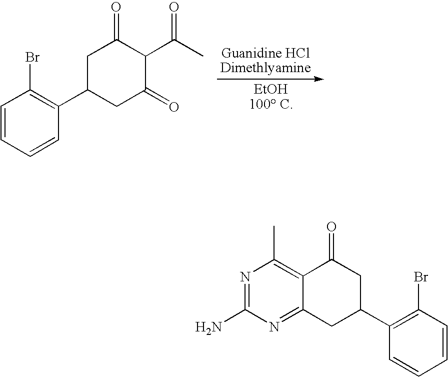

2-Amino-7-(2-bromophenyl)-7,8-dihydro-4-methylquinazolin-5(6H)-one: Combined 8.10 g (0.026 mol) of the compound prepared in step 4 with 20 mL of anhydrous ethanol. A solution of dimethyl amine in ethanol (33%, 32 mL, 0.18 mol) was added and the mixture was heated to 100° C. for 1 h. The reaction mixture was cooled to room temperature and 6.3 g (0.066 mol) of guanidine hydrochloride was added. The reaction was heated for 16 h at 100° C. After cooling the reaction mixture room temperature, the resulting solids were collected by filtration and washed with cold ethanol. Further drying in vacuo afforded 6.0 g (69% yield) of the title compound as a white solid.

### Example 2

Representative Method for Synthesizing 2-Amino-quinazolin-5-one Compounds: Method B

In this example, a method for making representative compounds of the invention (Method B) is described.

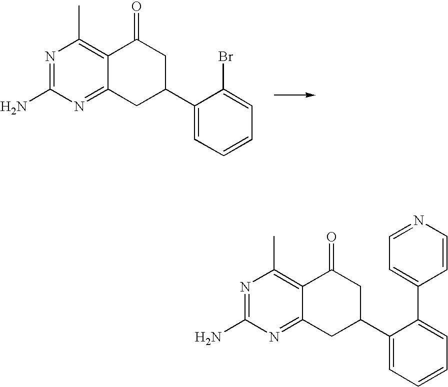

2-Amino-7,8-dihydro-4-methyl-7-(2-(pyridin-4-yl)phenyl)quinazolin-5(6H)-one: A small scintillation vial was charged with 2-amino-7-(2-bromophenyl)-7,8-dihydro-4-methylquinazolin-5(6H)-one (12 mg, 0.036 mmol, prepared as described in Method A), 4-tributylstannylpyridine (21 mg, 0.058 mmol), diisopropylamine (23 μl, 0.18 mmol), and DMF (1 ml). Nitrogen was then bubbled through the solution for 5 minutes. 1,1′-Bis(diphenylphosphino)ferrocene palladium (II) chloride (7 mg, 0.009 mmol) was then added and vial sealed and heated to 80° C. in an oil bath overnight. The solution was then cooled to room temperature, shaken with hexanes, and phases separated. The DMF phase was then purified via reverse phase HPLC to afford 2-amino-7,8-dihydro-4-methyl-7-(2-(pyridin-4-yl)phenyl)quinazolin-5(6H)-one (4.3 mg). MS: MH⁺=331.

### Example 3

Representative Method for Synthesizing 2-Amino-quinazolin-5-one Compounds: Method C

In this example, a method for making representative compounds of the invention (Method C) is described.

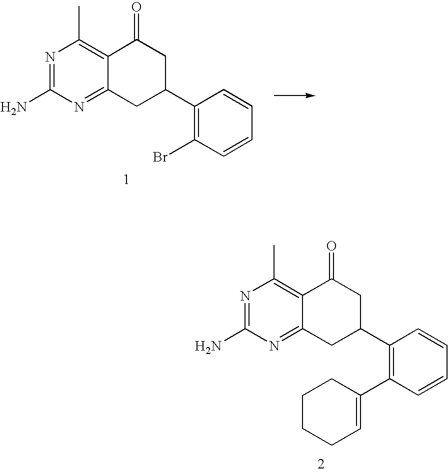

Pd(dppf)₂Cl₂ (0.08 eq) was added to a 0.1 M solution of compound 1 (1.0 eq, prepared as described in Method A), cyclohexen-1-yl-boronic acid (2.0 eq), and potassium carbonate (2.0 M in water, 1.6 eq) in N,N-dimethylacetamide. The reaction mixture was purged with argon and was microwaved at 150° C. for 10 min. The reaction mixture was diluted with ethyl acetate and washed successively with saturated sodium metabisulfite and brine. The organic phase was dried over sodium sulfate, filtered, and concentrated. The residue was purified by reverse-phase HPLC to give product 2. ES/MS: m/z 334 (MH⁺). C₂₁H₂₃N₃O=333 g/mol.

### Example 4

Representative Method for Synthesizing 2-Amino-quinazolin-5-one Compounds: Method D

In this example, a method for making representative compounds of the invention (Method D) is described.

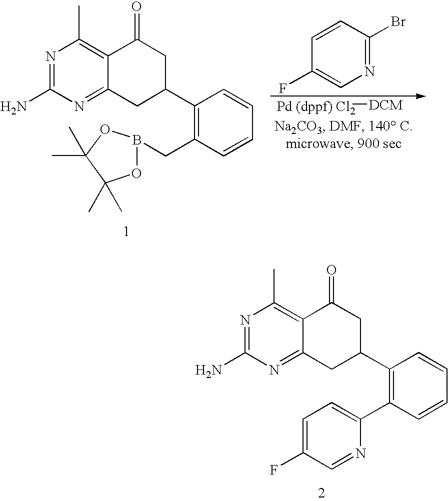

A solution of compound 1 (40 mg, 0.11 mmol), 2-bromo-5-fluoropyridine (40 mg, 0.23 mmol), and Pd catalyst (9 mg, 0.01 mmol) in DMF (3 mL) and Na₂CO₃ (100 μL, 2 M aq) was heating in a microwave for 900 seconds at 120° C. Upon cooling the reaction mixture was poured into 10 mL water and extracted with ethyl acetate (3×). The combined organics were washed with water and then concentrated. The resulting residue was purified by reverse phase HPLC to yield 3 mg of the product 2 as a TFA salt (Rt=1.997, m/z=349.3).

### Example 5

Representative Method for Synthesizing 2-Amino-quinazolin-5-one Compounds: Method E

In this example, a method for making representative compounds of the invention (Method E) is described.

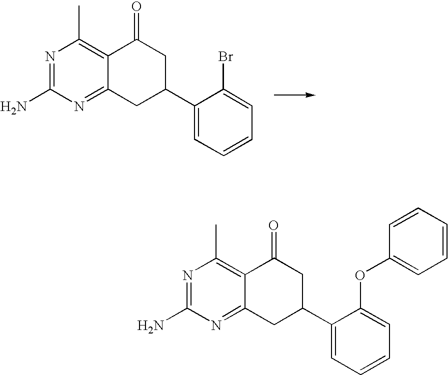

2-Amino-7,8-dihydro-4-methyl-7-(2-phenoxyphenyl)quinazolin-5(6H)-one: A scintillation vial was charged with 2-amino-7-(2-bromophenyl)-7,8-dihydro-4-methylquinazolin-5(6H)-one (50 mg, 0.151 mmol, prepared as described in Method A), phenol (28 mg, 0.301 mmol), cesium carbonate (98 mg, 0.301 mmol), N-methylpyrrolidinone (1 ml) and copper (I) iodide (2 mg, 0.01 mmol). The vial was then flushed with nitrogen and sealed and placed in an oil bath at 145° C. for 24 hours. The reaction mixture was then cooled to room temperature and diluted with water and ethyl acetate and filtered through Celite. Layers were then separated and aqueous extracted with ethyl acetate. The organic layers were then combined and washed with brine, dried with sodium sulfate, filtered and stripped to a black oil. The oil was then purified by reverse phase HPLC to yield 2-amino-7,8-dihydro-4-methyl-7-(2-phenoxyphenyl)quinazolin-5(6H)-one. MS: MH⁺=346.

### Example 6

Representative Method for Synthesizing 2-Amino-quinazolin-5-one Compounds: Method F

In this example, a method for making representative compounds of the invention (Method F) is described.

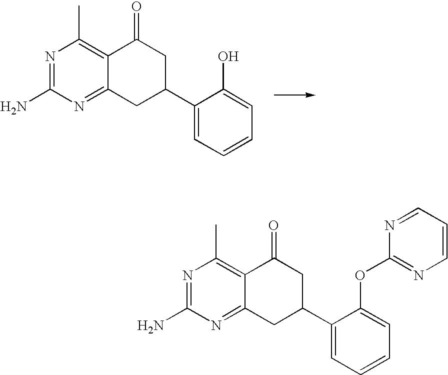

2-Amino-7,8-dihydro-4-methyl-7-(2-(pyrimidin-2-yloxy)phenyl)quinazolin-5(6H)-one: A scintillation vial was charged with 2-amino-7,8-dihydro-7-(2-hydroxyphenyl)-4-methylquinazolin-5(6H)-one (23 mg, 0.086 mmol, prepared as described in Method A), 2-chloropyrimidine (20 mg, 0.171 mmol), potassium carbonate (24 mg, 0.171 mmol)(previously flamed dried in vacuo), and DMSO (1 ml). The vial was then flushed with nitrogen, sealed, and placed in an oil bath at 135° C. for 24 hours. The reaction mixture was then diluted with water and extracted with ethyl acetate. The organic layer was then washed with a saturated solution of sodium bicarbonate, brine, and dried with potassium carbonate, filtered and concentrated in vacuo. To this oil was added ethanol (1 mL), heated to reflux, cooled to room temperature and then scratched with a glass rod. A crystalline product was then collected via vacuum filtration to afford of 2-amino-7,8-dihydro-4-methyl-7-(2-(pyrimidin-2-yloxy)phenyl)quinazolin-5(6H)-one. MS: MH⁺=348.

### Example 7

Representative Method for Synthesizing 2-Amino-quinazolin-5-one Compounds: Method G

In this example, a method for making representative compounds of the invention (Method G) is described.

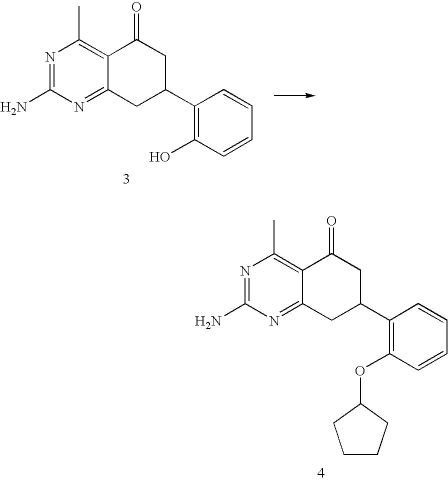

To a solution of cyclopentanol (2.0 eq) in THF at 0° C. under argon was added triphenylphosphine (2.0 eq). The resulting mixture was stirred at 0° C. for 30 min and a clear solution was formed. Diethyl azodicarboxylate (2.0 eq) was slowly added to the reaction solution at 0° C. and the resulting yellow solution was stirred at 0° C. for 1 hr. Compound 3 (1.0 eq, prepared as described in Method K) in THF was added. Reaction mixture was stirred at 0° C. for 1 hr and at ambient temperature for 10 hr. LCMS indicated that reaction was complete. Volatiles were removed under reduced pressure. The residue was purified by reverse-phase HPLC to give final product 4. ES/MS: m/z 338 (MH⁺). C₂₀H₂₃N₃O₂=337 g/mol.

### Example 8

Representative Method for Synthesizing 2-Amino-quinazolin-5-one Compounds: Method H

In this example, a method for making representative compounds of the invention (Method H) is described.

Step 1:

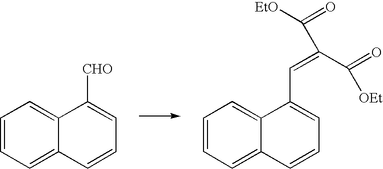

1-Napthaldehyde (100 mmol), diethyl malonate (100 mmol), and benzoic acid (2 mmol) are dissolved in 50 ml of anhydrous toluene. When this mixture begins to reflux, piperidine (2 mmol) is added. Reflux is continued for 5 hours while water is removed from the reaction via a Dean-Stark trap.

Solution is cooled and washed with water and saturated NaCl solution. The organic layer is separated, dried over Na₂SO₄ and evaporated. The crude product is purified by flash column chromatography (silica gel, 3:1 hexanes/ethyl acetate mixture) [adapted from JMC, 33, 2385-2393; 1990]

Step 2:

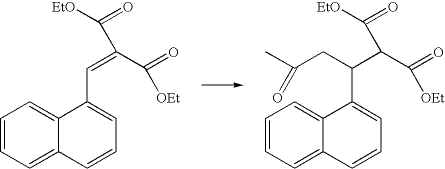

L-Proline (20 mol %) is added to a solution of diethyl 2-(α-napthylmethylene) malonate (1 mmol) in DMSO/acetone (4:1, 10 ml) and the mixture is stirred for 24 h at room temperature. The reaction mixture was treated with saturated ammonium chloride solution and the product was extracted with diethyl ether, dried over sodium sulfate, and evaporated. Purification by flash column chromatography (silica gel, 3:1 hexanes/ethyl acetate mixture) afforded the corresponding Michael adduct. [adapted from JACS, 123(22), 5260-5267; 2001]

Step 3:

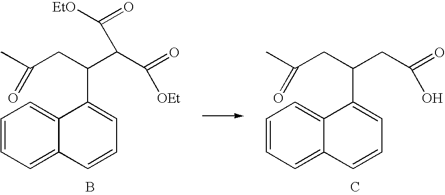

Compound prepared in step 2 (10 mmol) is refluxed overnight in a mixture of 10 ml glacial acetic acid, 6 ml water, and 5 ml concentrated hydrochloric acid. Reaction mixture is then cooled, diluted with water and extracted with ethyl acetate. Organic layer is separated, washed with saturated NaCl solution, dried over Na₂SO₄ and evaporated to give product.

Step 4:

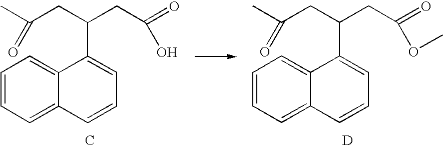

To carboxylic acid C (1 mmol) and methanol (5 ml) was added concentrated hydrochloric acid (0.5 ml). Solution refluxed for 3 hours. Solvent was evaporated to give the methyl ester.

Step 5:

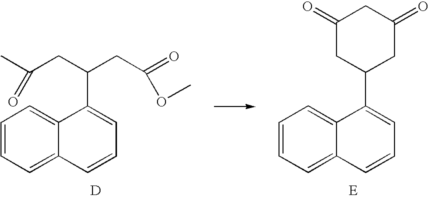

Compound prepared in step 4 (2 mmol), methanol (4 ml), and 1 ml of a 4M NaOMe solution in methanol were placed in a 5 ml microwave reaction vial and briefly degassed with argon. Tube was sealed and heated to 90° C. for 600 sec. Reaction mixture poured into a saturated ammonium chloride solution and extracted with ethyl acetate. Organic layer separated, washed with water, dried over Na₂SO₄ and evaporated to give product as a solid foam.

Step 6: Followed procedure in Method A, steps 3-5 to produce the final compound.

### Example 9

Representative Method for Synthesizing 2-Amino-quinazolin-5-one Compounds: Method I

In this example, a method for making representative compounds of the invention (Method I) is described.

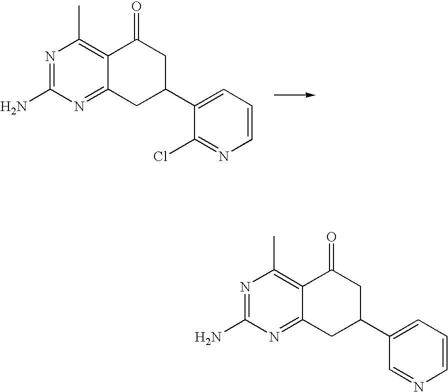

2-Amino-7,8-dihydro-4-methyl-7-(pyridin-3-yl)quinazolin-5(6H)-one: A glass Parr vessel was charged with 2-amino-7-(2-chloropyridin-3-yl)-7,8-dihydro-4-methylquinazolin-5(6H)-one (12 mg, 0.04 mmol), methanol (2 mL) and palladium on carbon (5 mg) in methanol (1 mL). Vessel was then placed on Parr apparatus and a hydrogen atmosphere charged to 50 psi. The solution was allowed to shake for 48 hours at room temperature. The reaction mixture was then filtered through Celite and concentrated in vacuo to yield the title compound as a white solid. MS: MH⁺=255.

### Example 10

Representative Method for Synthesizing 2-Amino-quinazolin-5-one Compounds: Method J

In this example, a method for making representative compounds of the invention (Method J) is described.

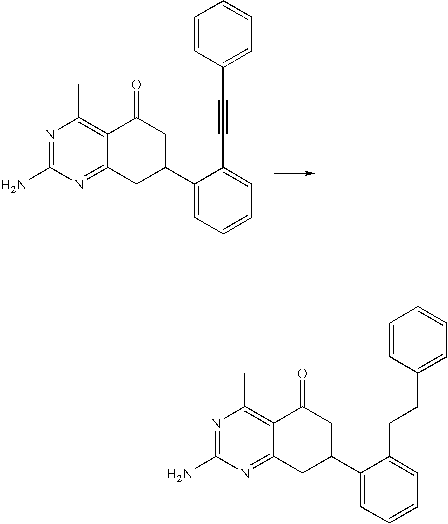

2-Amino-7,8-dihydro-4-methyl-7-(2-phenethylphenyl)quinazolin-5(6H)-one: A glass Parr vessel was charged with 2-amino-7,8-dihydro-4-methyl-7-(2-(2-phenylethynyl)phenyl)quinazolin-5(6H)-one (21 mg, 0.06 mmol), methanol (4 ml) and palladium on carbon (5 mg) in methanol (1 ml). Vessel was shaken under 50 psi hydrogen, 24 hours at room temperature. Mixture was then filtered through celite, concentrated in vacuo and purified by reverse phase HPLC to yield 2-amino-7,8-dihydro-4-methyl-7-(2-phenethyl)quinazolin-5(6H)-one. MS: MH⁺=358.

### Example 11

Representative Method for Synthesizing 2-Amino-quinazolin-5-one Compounds: Method K

In this example, a method for making representative compounds of the invention (Method K) is described.

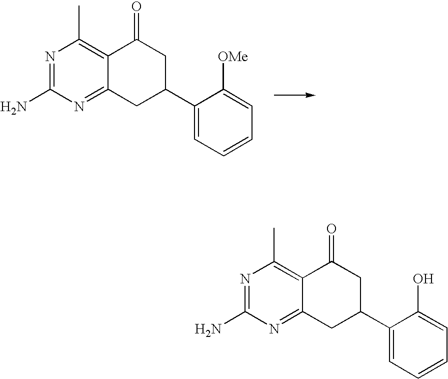

2-Amino-7,8-dihydro-7-(2-hydroxyphenyl)-4-methylquinazotin-5(6H)-one: A glass tube was charged with 2-amino-7,8-dihydroxy-7-(2-methoxyphenyl)-4-methylquinazolin-5(6H)-one (270 mg, 0.954 mmol), 4-aminothiophenol (125 mg, 1.05 mmol), potassium fluoride (6 mg, 0.095 mmol) and N-methylpyrrolidinone (10 ml) and sealed. Tube was then placed in an oil bath at 200° C. for 24 hours. Reaction mixture was diluted with citric acid (10% w/w) and extracted with ethyl acetate. Organic layer was then washed with water, brine and dried with sodium sulfate, filtered and concentrated in vacuo to yield 2-amino-7,8-dihydro-7-(2-hydroxyphenyl)-4-methylquinazolin-5(6H)-one. MS: MH⁺=270.

### Example 12

Representative Method for Synthesizing 2-Amino-quinazolin-5-one Compounds: Method L

In this example, a method for making representative compounds of the invention (Method L) is described.

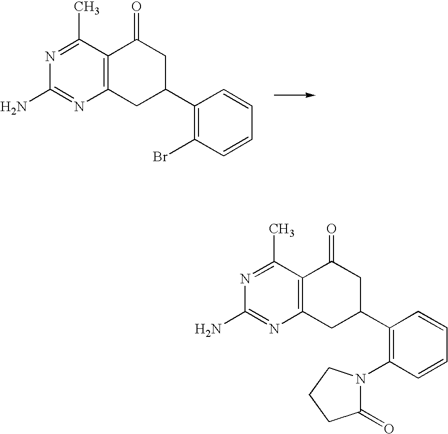

2-Amino-7,8-dihydro-4-methyl-7-(2-(2-oxopyrrolidin-1-yl)phenyl)quinazolin-5(6H)-one: To a suspension of aryl bromide (66 mg, 0.20 mmol, prepared as described in Method A) in anhydrous toluene (0.50 mL) under an inert gas atmosphere was added copper(I) iodide (1.9 mg, 0.010 mmoL), 2-pyrrolidinone (10 μL, 0.204 mmol), flame-dried potassium carbonate (55 mg, 0.40 mmol), and N,N′-dimethylethylenediamine (2.2 μL, 0.020 mmol). The suspension was refluxed over 48 h. The mixture was diluted with ethyl acetate and filtered. The supernatant was concentrated and purified by reverse-phase HPLC to give the desired compound. ES/MS: m/z 337 (MH⁺). Retention time=1.79 min.

### Example 13

Representative Method for Synthesizing 2-Amino-quinazolin-5-one Compounds: Method M

In this example, a method for making representative compounds of the invention (Method M) is described.

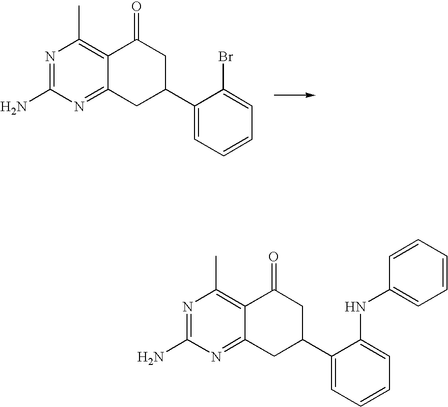

2-Amino-7,8-dihydro-4-methyl-7-(2-phenylaminophenyl)quinazolin-5(6H)-one: To a suspension of aryl bromide (66 mg, 0.20 mmol, prepared as described in Method A) in anhydrous toluene under an inert gas atmosphere was added aniline (10 μL, 0.20 mmol), cesium carbonate (91 mg, 0.28 mmol), trisdibenzylidenedipalladium(0) chloroform adduct (9.3 mg, 0.045 mmol), and BINAP (3.8 mg, 0.060 mmol). The suspension was refluxed over 48 h. The mixture was diluted with ethyl acetate and filtered. The supernatant was concentrated and purified by reverse-phase HPLC to give the desired compound. ES/MS: m/z 345 (MH⁺). Retention time=2.61 min.

### Example 14

Representative Method for Synthesizing 2-Amino-quinazolin-5-one Compounds: Method N

In this example, a method for making representative compounds of the invention (Method N) is described.

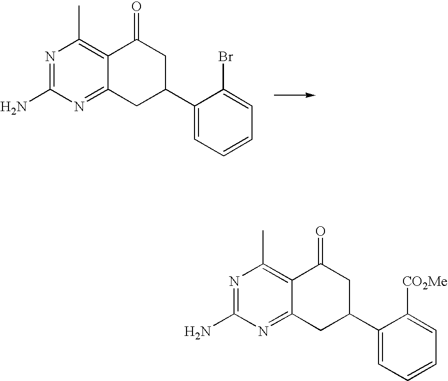

Methyl 2-(2-amino-5,6,7,8-tetrahydro-4-methyl-5-oxoquinazolin-7-yl)benzoate: 2-Amino-7-(2-bromophenyl)-4-methyl-7,8-dihydro-6H-quinazolin-5-one (prepared by Method A) was heated in methanol with Pd(BiNap)Cl₂ (2 mole %) and triethylamine (1 eq) under carbon monoxide (85 psig) at 140° C. for 12 hrs. The reaction mixture was concentrated and purified by reverse-phase HPLC to afford the title compound.

### Example 15

Representative Method for Synthesizing 2-Amino-quinazolin-5-one Compounds: Method O

In this example, a method for making representative compounds of the invention (Method O) is described.

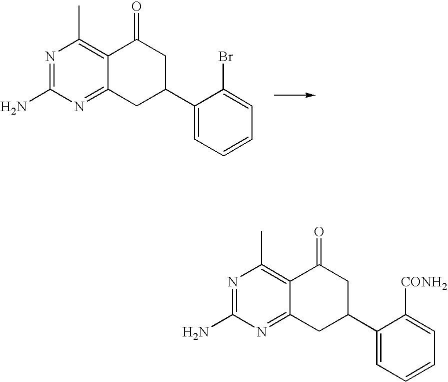

2-(2-Amino-5,6,7,8-tetrahydro-4-methyl-5-oxoquinazolin-7-yl)benzamide: 2-Amino-7-(3-bromo-phenyl)-4-methyl-7,8-dihydro-6H-quinazolin-5-one (prepared by Method A) was heated in formamide with Pd(dppf)Cl₂ (2 mole %) and DMAP (1 eq) under carbon monoxide (85 psig) at 100° C. for 12 hrs. The reaction mixture was concentrated and purified by reverse-phase HPLC to afford the title compound.

### Example 16

Representative Method for Synthesizing 2-Amino-quinazolin-5-one Compounds: Method P

In this example, a method for making representative compounds of the invention (Method P) is described.

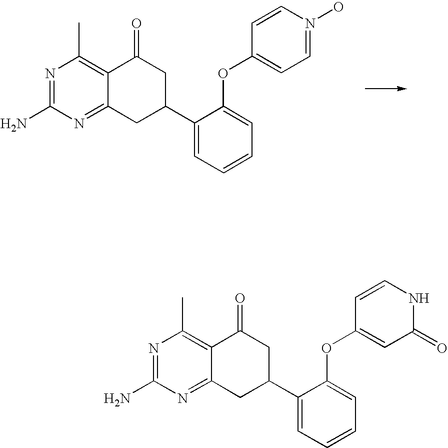

7-(2-(1,2-Dihydro-2-oxopyridin-4-yloxy)phenyl)-2-amino-7,8-dihydro-4-methylquinazolin-5(6h)-one: A solution of 2-amino-7,8-dihydro-4-methyl-7-(2-([4-N-oxopyridyl]-loxy)phenyl)quinazolin-5(6H)-one (65 mg, 0.17 mmol, prepared as described in Method F) in acetic anhydride (1 ml) was heated at 140° C. in an oil bath for 3 hours. The reaction mixture was then placed cooled to room temperature and to it was added water (1 mL), methanol (1 ml) and ammonia in isopropanol (2.0M solution) (1 mL). Vessel was sealed and heated for 48 hours in an oil bath at 65° C. Solvent was then removed in vacuo and resulting oil was purified by reverse phase HPLC to afford 7-(2-(1,2-dihydro-2-oxopyridin-4-yloxy)phenyl)-2-amino-7,8-dihydro-4-methylquinazolin-5(6h)-one (2.3 mg). MS: MH⁺=363.

### Example 17

Representative Method for Synthesizing 2-Amino-quinazolin-5-one Compounds: Method Q

In this example, a method for making representative compounds of the invention (Method Q) is described.

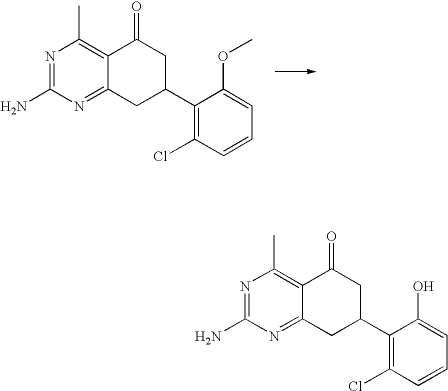

2-Amino-7-(2-chloro-6-hydroxyphenyl)-7,8-dihydro-4-methylquinazolin-5(6H)-one: The mixture of 2-amino-7-(2-chloro-6-methoxyphenyl)-7,8-dihydro-4-methylquinazolin-5(6H)-one (20 mg, 1.0 eq, prepared as described in Method A), 4-aminothiophenol (9.0 mg, 1.1 eq), KF (0.3 mg, 0.1 eq) in 1 ml NMP was heated to 200° C. in an oil bath for 15 h. The reaction mixture was diluted with ethyl acetate and washed successively with 10% citric acid and brine. The organic phase was dried over sodium sulfate, filtered, and concentrated. The residue was purified by reverse-phase HPLC to give final product (8.2 mg, yield 43%). ES/MS: m/z 303/305 (MH⁺). C₁₅H₁₄ClN₃O₂=303 g/mol.

### Example 18

Representative Method for Synthesizing 2-Amino-quinazolin-5-one Compounds: Method R

In this example, a method for making representative compounds of the invention (Method R) is described.

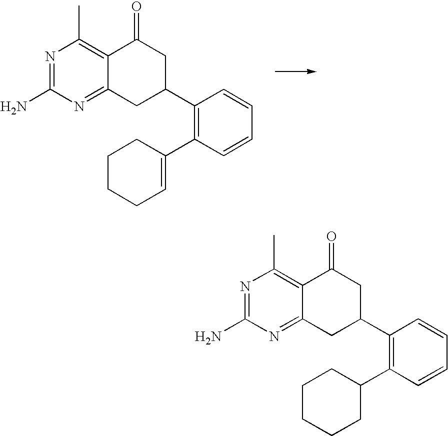

2-Amino-7-(2-cyclohexylphenyl)-7,8-dihydro-4-methylquinazolin-5(6H)-one: A solution of 2-amino-7-(2-cyclohexenylphenyl)-7,8-dihydro-4-methylquinazolin-5(6H)-one (18 mg, 1.0 eq, prepared as described in Method C) in 10 ml methanol and DIEA (7.0 mg, 1.0 eq) was treated with palladium-on-carbon (20 wt %, 3.6 mg) and stirred under 65 psi of hydrogen for 18 h at ambient temperature. Reaction suspension was filtered through Celite. The filter cake was rinsed with methanol and the combined methanol solution was concentrated under reduced pressure to give oil residue, which was purified by reverse-phase HPLC to give final product (4.0 mg, yield 45% based on 50% conversion of the reaction). ES/MS: m/z 336 (MH⁺). C₂₁H₂₅N₃O=335 g/mol.

### Example 19

Representative Method for Synthesizing 2-Amino-quinazolin-5-one Compounds: Method R

In this example, a method for making representative compounds of the invention (Method R) is described.

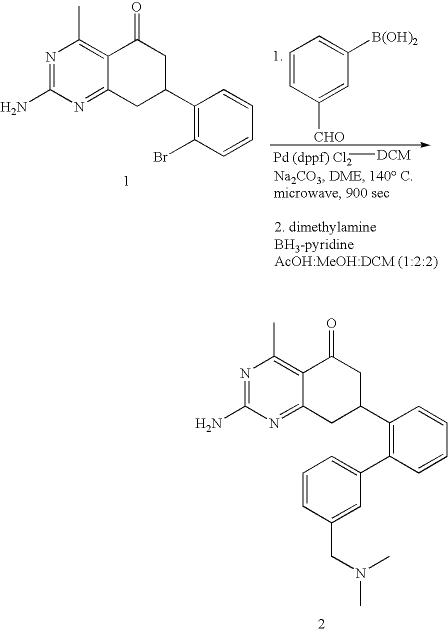

The Suzuki coupling was carried out as previously described in Method C on 100 mg scale (0.3 mmol) and taken through to the next step with no purification (Rt=2.25 min, m/z=358.3). The intermediate aldehyde (0.3 mmol) is dissolved a mixture of acetic acid, methanol and dichloromethane (1:2:2) whereupon dimethylamine in ethanol (100 μL, 1M solution), and borane-pyridine (100 μL, 8M solution) are added and left to shake overnight. LCMS shows 50% conversion to the desired product at this time. The solvent is evaporated and the resulting residue was purified by reverse phase HPLC to yield 12.5 mg of product 2 as a TFA salt (Rt=1.801 min, m/z=387.3).

### Example 20

Representative Method for Synthesizing 2-Amino-quinazolin-5-one Compounds: Method S

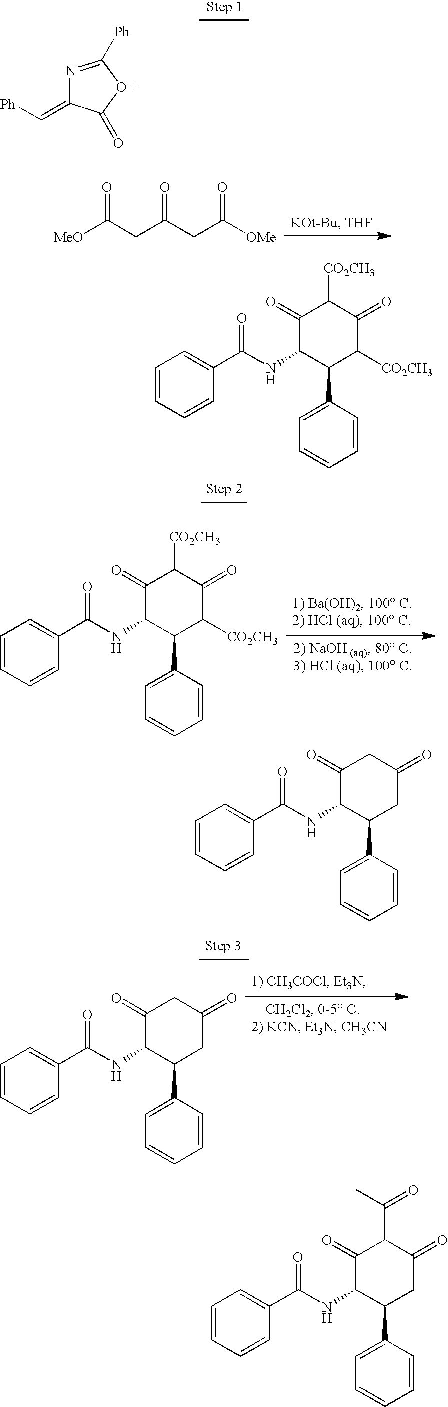

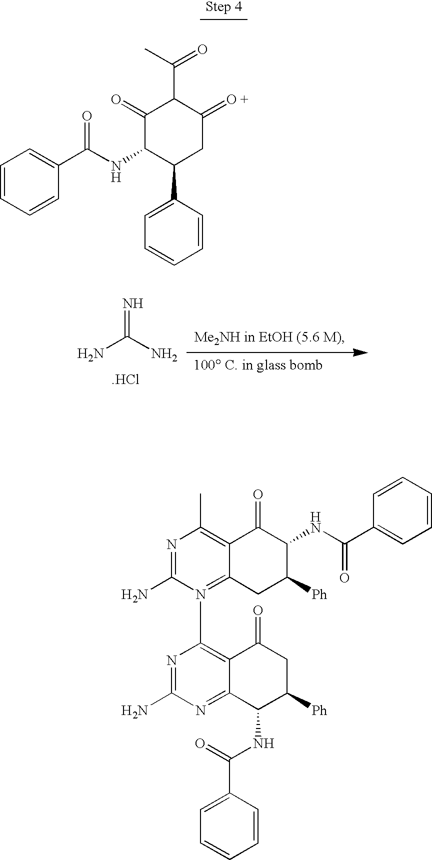

### Example 21

Representative 2-Amino-4-methyldihydroquinazolinone Compounds

Representative 2-amino-4-methyldihydroquinazolinone compounds are shown in Tables I and II. Expermimental data and synthesis information for the compounds in Table I is given in Table Ia.

TABLE I

| | | Hsp90 IC50 | |
|---|---|---|---|
| | | Range A = | |
| | | >10 μM, B = | |
| | | 1-10 μM, C = | |
| Compound | Structure | <1 μM | Name |
| 1 | 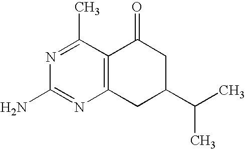 | A | 2-amino-7-isopropyl-4- methyl-7,8- dihydroquinazolin-5(6H)- one |
| 2 | 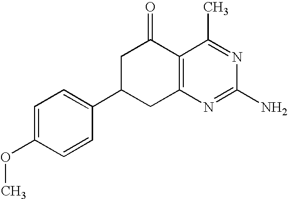 | B | 2-amino-7-(4- methoxyphenyl)-4- methyl-7,8- dihydroquinazolin-5(6H)- one |
| 3 | 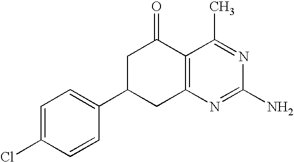 | C | 2-amino-7-(4- chlorophenyl)-4-methyl- 7,8-dihydroquinazolin- 5(6H)-one |
| 4 | 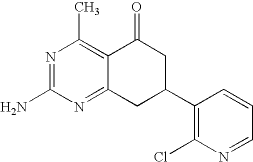 | A | 2-amino-7-(2- chloropyridin-3-yl)-4- methyl-7,8- dihydroquinazolin-5(6H)- one |
| 5 | 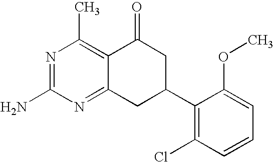 | B | 2-amino-7-(2-chloro-6- methoxy-phenyl)-4- methyl-7,8-dihydro-6H- quinazolin-5-one |
| 6 | 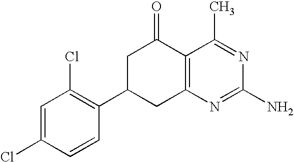 | B | 2-amino-7-(2,4- dichlorophenyl)-4- methyl-7,8- dihydroquinazolin-5(6H)- one |
| 7 | 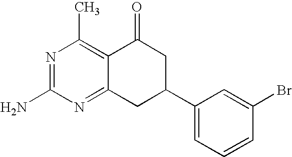 | B | 2-amino-7-(3- bromophenyl)-4-methyl- 7,8-dihydroquinazolin- 5(6H)-one |
| 8 | 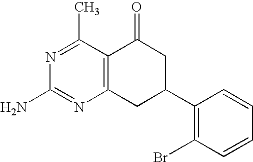 | B | 2-amino-7-(2- bromophenyl)-4-methyl- 7,8-dihydroquinazolin- 5(6H)-one |
| 9 | 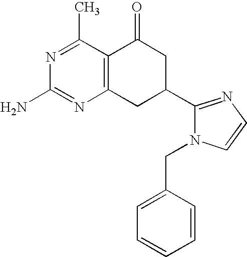 | C | 2-amino-7-(1-benzyl-1H- imidazol-2-yl)-4-methyl- 7,8-dihydro-6H- quinazolin-5-one |
| 10 | 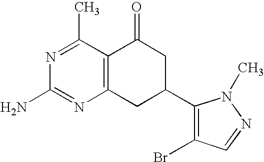 | A | 2-amino-7-(4-bromo-2- methyl-2H-pyrazol-3-yl)- 4-methyl-7,8-dihydro- 6H-quinazolin-5-one |
| 11 | 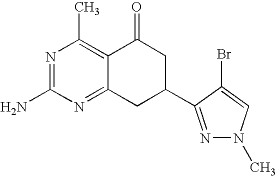 | A | 2-amino-7-(4-bromo-1- methyl-1H-pyrazol-3-yl)- 4-methyl-7,8-dihydro- 6H-quinazolin-5-one |
| 12 | 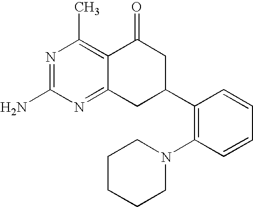 | C | 2-amino-4-methyl-7-(2- piperidin-1-yl-phenyl)- 7,8-dihydro-6H- quinazolin-5-one |
| 13 | 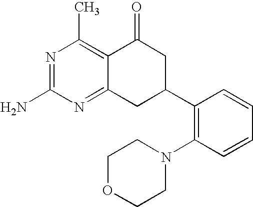 | C | 2-amino-4-methyl-7-(2- morpholin-4-yl-phenyl)- 7,8-dihydro-6H- quinazolin-5-one |
| 14 | 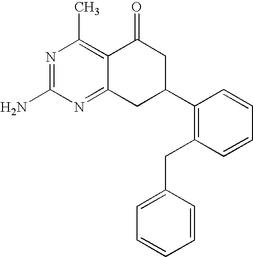 | C | 2-amino-7-(2-benzyl- phenyl)-4-methyl-7,8- dihydro-6H-quinazolin-5- one |
| 15 | 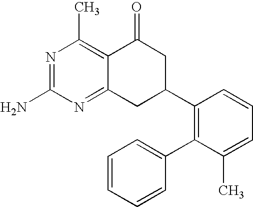 | B | 2-amino-4-methyl-7-(6- methyl-1,1′-biphenyl-2- yl)-7,8- dihydroquinazolin-5(6H)- one |
| 16 | 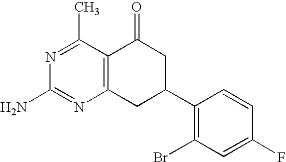 | C | 2-amino-7-(2-bromo-4- fluoro-phenyl)-4-methyl- 7,8-dihydro-6H- quinazolin-5-one |
| 17 | 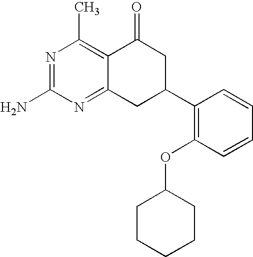 | C | 2-amino-7-[2- (cyclohexyloxy)phenyl]- 4-methyl-7,8- dihydroquinazolin-5(6H)- one |
| 18 | 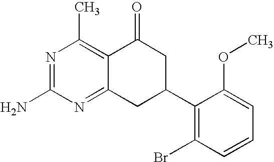 | B | 2-amino-7-(2-bromo-6- methoxy-phenyl)-4- methyl-7,8-dihydro-6H- quinazolin-5-one |
| 19 | 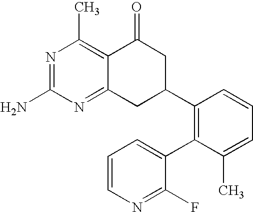 | B | 2-amino-7-[2-(2- fluoropyridin-3-yl)-3- methylphenyl]-4-methyl- 7,8-dihydroquinazolin-5(6H)-one |
| 20 | 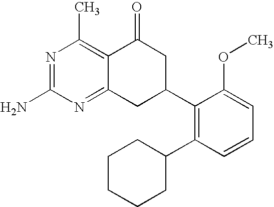 | A | 2-amino-7-(2-cyclohexyl- 6-methoxyphenyl)-4- methyl-7,8- dihydroquinazolin-5(6H)- one |
| 21 | 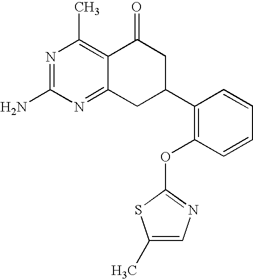 | C | 2-amino-4-methyl-7-[2- (5-methyl-thiazol-2- yloxy)-phenyl]-7,8- dihydro-6H-quinazolin-5- one |
| 22 | 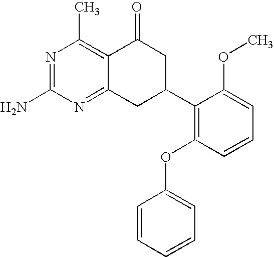 | B | 2-amino-7-(2-methoxy-6- phenoxyphenyl)-4- methyl-7,8- dihydroquinazolin-5(6H)- one |
| 23 | 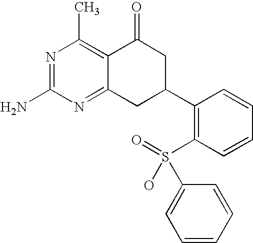 | A | 2-amino-7-(2- benzenesulfonyl-phenyl)- 4-methyl-7,8-dihydro- 6H-quinazolin-5-one |
| 24 | 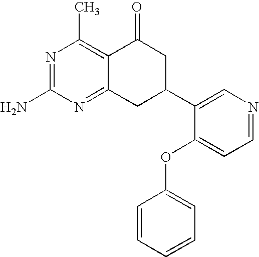 | C | 2-Amino-4-methyl-7-(4- phenoxy-pyridin-3-yl)- 7,8-dihydro-6H- quinazolin-5-one |
| 25 | 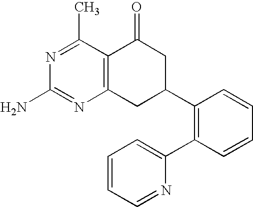 | C | 2-amino-4-methyl-7-(2- pyridin-2-ylphenyl)-7,8- dihydroquinazolin-5(6H)- one |
| 26 | 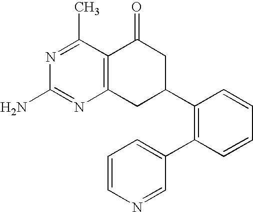 | C | 2-amino-4-methyl-7-(2- pyridin-3-ylphenyl)-7,8- dihydroquinazolin-5(6H)- one |
| 27 | 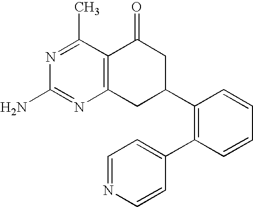 | B | 2-amino-4-methyl-7-(2- pyridin-4-ylphenyl)-7,8- dihydroquinazolin-5(6H)- one |
| 28 | 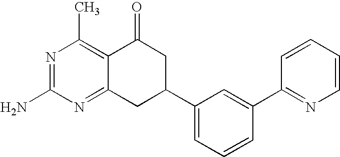 | A | 2-amino-4-methyl-7-(3- pyridin-2-ylphenyl)-7,8- dihydroquinazolin-5(6H)- one |
| 29 | 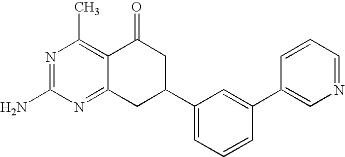 | A | 2-amino-4-methyl-7-(3- pyridin-3-ylphenyl)-7,8- dihydroquinazolin-5(6H)- one |
| 30 | 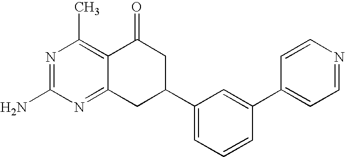 | A | 2-amino-4-methyl-7-(3- pyridin-4-ylphenyl)-7,8- dihydroquinazolin-5(6H)- one |
| 31 | 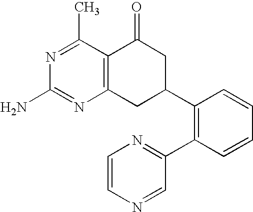 | C | 2-amino-4-methyl-7-(2- pyrazin-2-ylphenyl)-7,8- dihydroquinazolin-5(6H)- one |
| 32 | 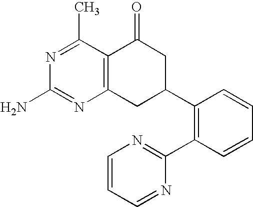 | C | 2-amino-4-methyl-7-(2- pyrimidin-2-ylphenyl)- 7,8-dihydroquinazolin- 5(6H)-one |
| 33 | 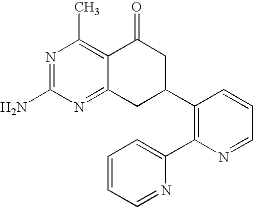 | B | 2-amino-7-(2,2′- bipyridin-3-yl)-4-methyl- 7,8-dihydroquinazolin- 5(6H)-one |
| 34 | 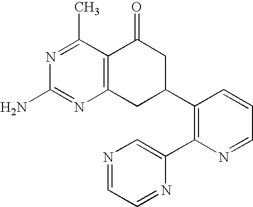 | B | 2-amino-4-methyl-7-(2- pyrazin-2-ylpyridin-3-yl)- 7,8-dihydroquinazolin- 5(6H)-one |
| 35 | 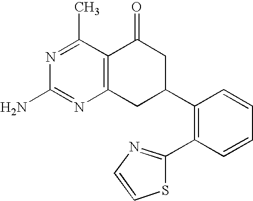 | C | 2-amino-4-methyl-7-[2- (1,3-thiazol-2-yl)phenyl]- 7,8-dihydroquinazolin- 5(6H)-one |
| 36 | 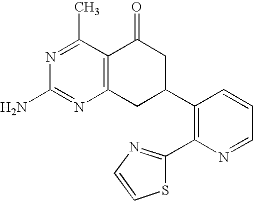 | B | 2-amino-4-methyl-7-[2- (1,3-thiazol-2-yl)pyridin- 3-yl]-7,8- dihydroquinazolin-5(6H)- one |
| 37 | 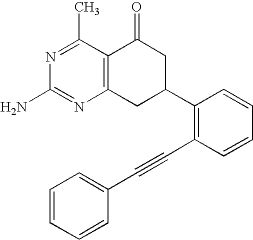 | A | 2-amino-4-methyl-7-[2- (phenylethynyl)phenyl]- 7,8-dihydroquinazolin- 5(6H)-one |
| 38 | 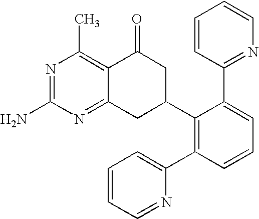 | C | 2-amino-7-(2,6-dipyridin- 2-ylphenyl)-4-methyl- 7,8-dihydroquinazolin- 5(6H)-one |
| 39 | 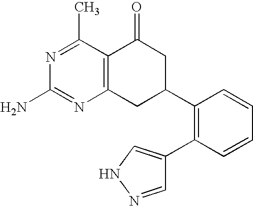 | B | 2-amino-4-methyl-7-[2- (1H-pyrazol-4- yl)phenyl]-7,8- dihydroquinazolin-5(6H)- one |
| 40 | 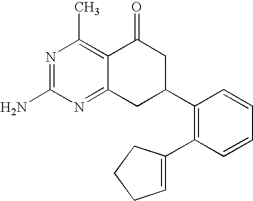 | C | 2-amino-7-(2-cyclopent- 1-en-1-ylphenyl)-4- methyl-7,8- dihydroquinazolin-5(6H)- one |
| 41 | 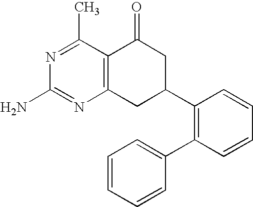 | C | 2-amino-7-(1,1′- biphenyl-2-yl)-4-methyl- 7,8-dihydroquinazolin- 5(6H)-one |
| 42 | 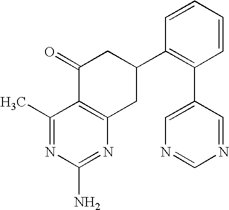 | C | 2-amino-4-methyl-7-(2- pyrimidin-5-ylphenyl)- 7,8-dihydroquinazolin- 5(6H)-one |
| 43 | 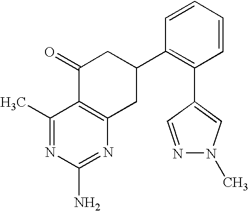 | B | 2-amino-4-methyl-7-[2- (1-methyl-1H-pyrazol-4- yl)phenyl]-7,8- dihydroquinazolin-5(6H)- one |
| 44 | 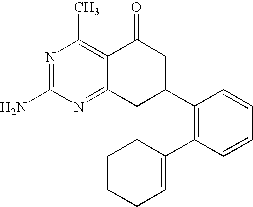 | C | 2-amino-7-(2-cyclohex-1- en-1-ylphenyl)-4-methyl- 7,8-dihydroquinazolin- 5(6H)-one |
| 45 | 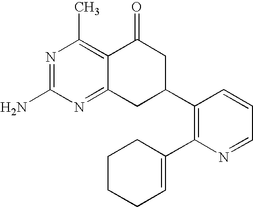 | C | 2-amino-7-(2-cyclohex-1- en-1-ylpyridin-3-yl)-4- methyl-7,8- dihydroquinazolin-5(6H)- one |
| 46 | 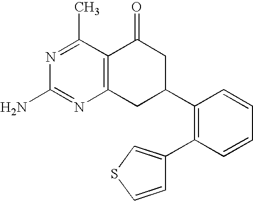 | C | 2-amino-4-methyl-7-(2- thien-3-ylphenyl)-7,8- dihydroquinazolin-5(6H)- one |
| 47 | 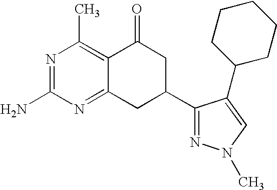 | C | 2-amino-7-(4-cyclohexyl- 1-methyl-1H-pyrazol-3- yl)-4-methyl-7,8-dihydro- 6H-quinazolin-5-one |
| 48 | 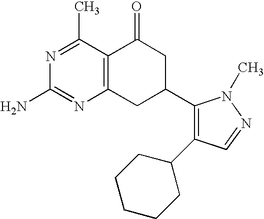 | C | 2-amino-7-(4-cyclohexyl- 2-methyl-2H-pyrazol-3- yl)-4-methyl-7,8-dihydro- 6H-quinazolin-5-one |
| 49 | 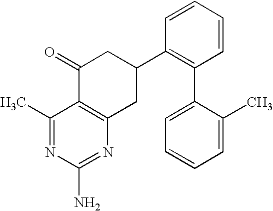 | C | 2-amino-4-methyl-7-(2′- methyl-1,1′-biphenyl-2- yl)-7,8- dihydroquinazolin-5(6H)- one |
| 50 | 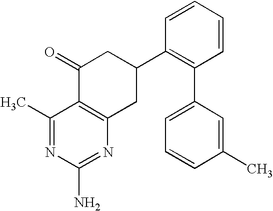 | B | 2-amino-4-methyl-7-(3′- methyl-1,1′-biphenyl-2- yl)-7,8- dihydroquinazolin-5(6H)- one |
| 51 | 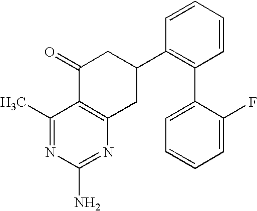 | C | 2-amino-7-(2′-fluoro- 1,1′-biphenyl-2-yl)-4- methyl-7,8- dihydroquinazolin-5(6H)- one |
| 52 | 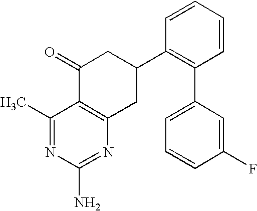 | C | 2-amino-7-(3′-fluoro- 1,1′-biphenyl-2-yl)-4- methyl-7,8- dihydroquinazolin-5(6H)- one |
| 53 | 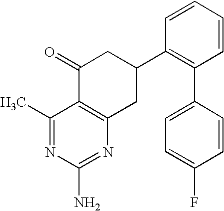 | C | 2-amino-7-(4′-fluoro- 1,1′-biphenyl-2-yl)-4- methyl-7,8- dihydroquinazolin-5(6H)- one |
| 54 | 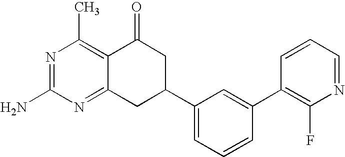 | A | 2-amino-7-(2′-fluoro- 1,1′-biphenyl-3-yl)-4- methyl-7,8- dihydroquinazolin-5(6H)- one |
| 55 | 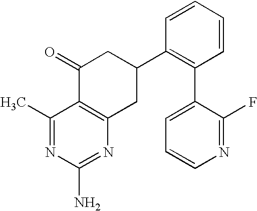 | C | 2-amino-7-[2-(2- fluoropyridin-3- yl)phenyl]-4-methyl-7,8- dihydroquinazolin-5(6H)- one |
| 56 | 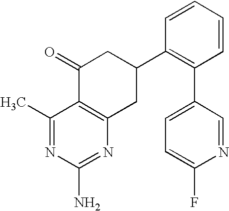 | C | 2-amino-7-[2-(6- fluoropyridin-3- yl)phenyl]-4-methyl-7,8- dihydroquinazolin-5(6H)- one |
| 57 | 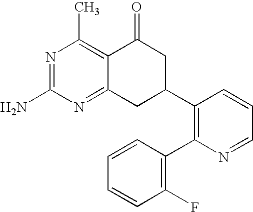 | C | 2-amino-7-[2-(2- fluorophenyl)pyridin-3- yl]-4-methyl-7,8- dihydroquinazolin-5(6H)- one |
| 58 | 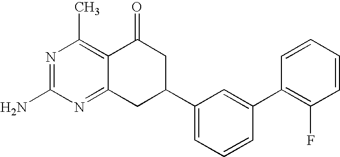 | A | 2-amino-7-[3-(2- fluoropyridin-3- yl)phenyl]-4-methyl-7,8- dihydroquinazolin-5(6H)- one |
| 59 | 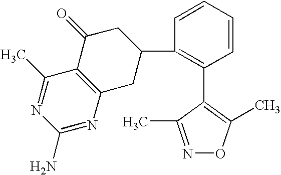 | B | 2-amino-7-[2-(3,5- dimethylisoxazol-4- yl)phenyl]-4-methyl-7,8- dihydroquinazolin-5(6H)- one |
| 60 | 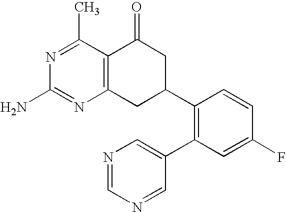 | C | 2-amino-7-(4-fluoro-2- pyrimidin-5-yl-phenyl)-4- methyl-7,8-dihydro-6H- quinazolin-5-one |
| 61 | 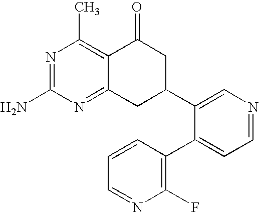 | C | 2-amino-7-(2-fluoro- [3,4′]bipyridinyl-3′-yl)-4- methyl-7,8-dihydro-6H- quinazolin-5-one |
| 62 | 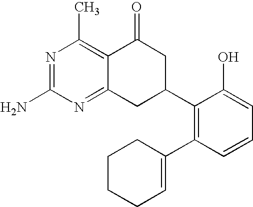 | C | 2-amino-7-(2-cyclohex-1- enyl-6-hydroxy-phenyl)- 4-methyl-7,8-dihydro- 6H-quinazolin-5-one |
| 63 | 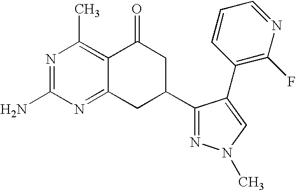 | C | 2-amino-7-[4-(2-fluoro- pyridin-3-yl)-1-methyl- 1H-pyrazol-3-yl]-4- methyl-7,8-dihydro-6H- quinazolin-5-one |
| 64 | 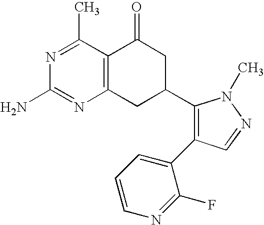 | B | 2-amino-7-[4-(2-fluoro- pyridin-3-yl)-2-methyl- 2H-pyrazol-3-yl]-4- methyl-7,8-dihydro-6H- quinazolin-5-one |
| 65 | 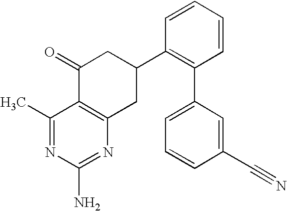 | B | 2′-(2-amino-4-methyl-5- oxo-5,6,7,8- tetrahydroquinazolin-7- yl)-1,1′-biphenyl-3- carbonitrile |
| 66 | 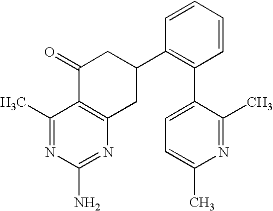 | B | 2-amino-7-[2-(2,6- dimethylpyridin-3- yl)phenyl]-4-methyl-7,8- dihydroquinazolin-5(6H)- one |
| 67 | 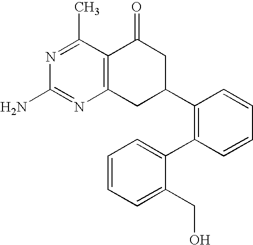 | C | 2-amino-7-[2′- (hydroxymethyl)-1,1′- biphenyl-2-yl]-4-methyl- 7,8-dihydroquinazolin- 5(6H)-one |
| 68 | 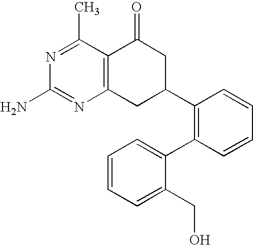 | C | 2-amino-7-[2′- (hydroxymethyl)-1,1′- biphenyl-2-yl]-4-methyl- 7,8-dihydroquinazolin- 5(6H)-one |
| 69 | 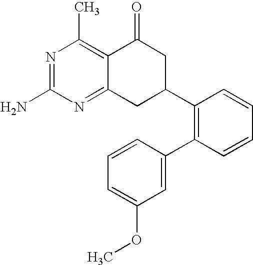 | B | 2-amino-7-(3′-methoxy- 1,1′-biphenyl-2-yl)-4- methyl-7,8- dihydroquinazolin-5(6H)- one |
| 70 | 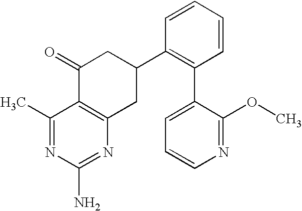 | C | 2-amino-7-[2-(2- methoxypyridin-3- yl)phenyl]-4-methyl-7,8- dihydroquinazolin-5(6H)- one |
| 71 | 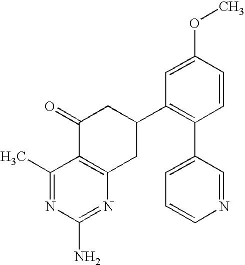 | C | 2-amino-7-(5-methoxy-2- pyridin-3-ylphenyl)-4- methyl-7,8- dihydroquinazolin-5(6H)- one |
| 72 | 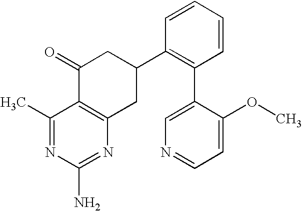 | B | 2-amino-7-[2-(4- methoxypyridin-3- yl)phenyl]-4-methyl-7,8- dihydroquinazolin-5(6H)- one |
| 73 | 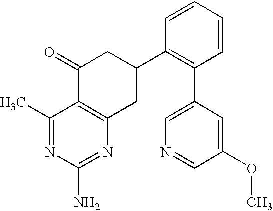 | B | 2-amino-7-[2-(5- methoxypyridin-3- yl)phenyl]-4-methyl-7,8- dihydroquinazolin-5(6H)- one |
| 74 | 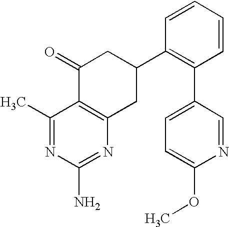 | B | 2-amino-7-[2-(6- methoxypyridin-3- yl)phenyl]-4-methyl-7,8- dihydroquinazolin-5(6H)- one |
| 75 | 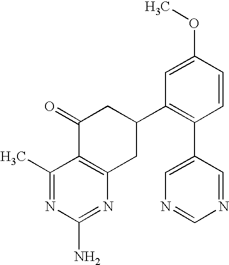 | B | 2-amino-7-(5-methoxy-2- pyrimidin-5-ylphenyl)-4- methyl-7,8- dihydroquinazolin-5(6H)- one |
| 76 | 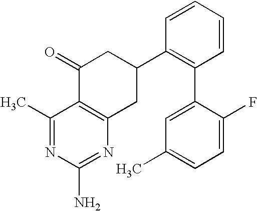 | C | 2-amino-7-(2′-fluoro-5′- methyl-1,1′-biphenyl-2- yl)-4-methyl-7,8- dihydroquinazolin-5(6H)- one |
| 77 | 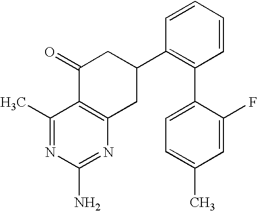 | C | 2-amino-7-(2′-fluoro-4′- methyl-1,1′-biphenyl-2- yl)-4-methyl-7,8- dihydroquinazolin-5(6H)- one |
| 78 | 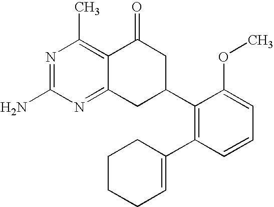 | C | 2-amino-7-(2-cyclohex-1- en-1-yl-6- methoxyphenyl)-4- methyl-7,8- dihydroquinazolin-5(6H)- one |
| 79 | 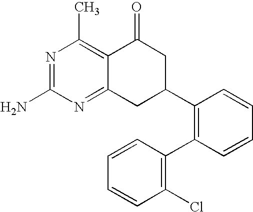 | C | 2-amino-7-(2′-chloro- 1,1′-biphenyl-2-yl)-4- methyl-7,8- dihydroquinazolin-5(6H)- one |
| 80 | 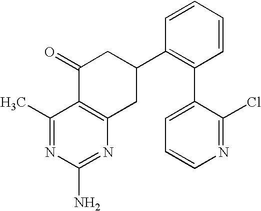 | C | 2-amino-7-[2-(2- chloropyridin-3- yl)phenyl]-4-methyl-7,8- dihydroquinazolin-5(6H)- one |
| 81 | 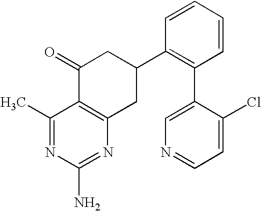 | C | 2-amino-7-[2-(4- chloropyridin-3- yl)phenyl]-4-methyl-7,8- dihydroquinazolin-5(6H)- one |
| 82 | 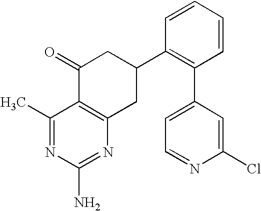 | B | 2-amino-7-[2-(2- chloropyridin-4- yl)phenyl]-4-methyl-7,8- dihydroquinazolin-5(6H)- one |
| 83 | 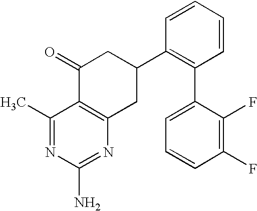 | C | 2-amino-7-(2′,3′- difluoro-1,1′-biphenyl-2- yl)-4-methyl-7,8- dihydroquinazolin-5(6H)- one |
| 84 | 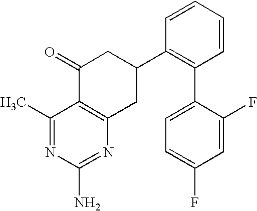 | C | 2-amino-7-(2′,4′- difluoro-1,1′-biphenyl-2- yl)-4-methyl-7,8- dihydroquinazolin-5(6H)- one |
| 85 | 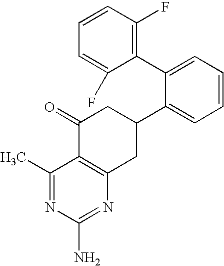 | C | 2-amino-7-(2′,6′- difluoro-1,1′-biphenyl-2- yl)-4-methyl-7,8- dihydroquinazolin-5(6H)- one |
| 86 | 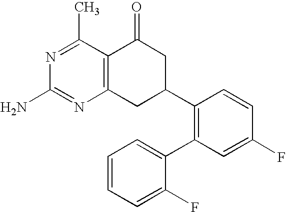 | C | 2-amino-7-(5,2′-difluoro- biphenyl-2-yl)-4-methyl- 7,8-dihydro-6H- quinazolin-5-one |
| 87 | 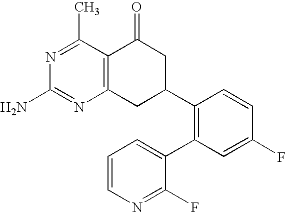 | C | 2-amino-7-[4-fluoro-2-(2- fluoropyridin-3- yl)phenyl]-4-methyl-7,8- dihydroquinazolin-5(6H)- one |
| 88 | 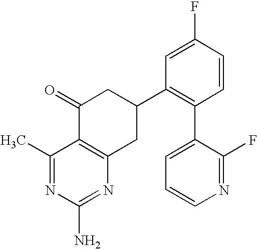 | C | 2-amino-7-[5-fluoro-2-(2- fluoropyridin-3- yl)phenyl]-4-methyl-7,8- dihydroquinazolin-5(6H)- one |
| 89 | 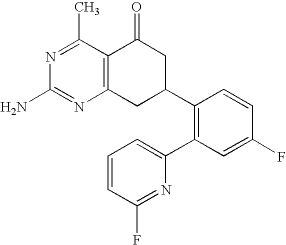 | C | 2-amino-7-[4-fluoro-2-(6- fluoro-pyridin-2-yl)- phenyl]-4-methyl-7,8- dihydro-6H-quinazolin-5- one |
| 90 | 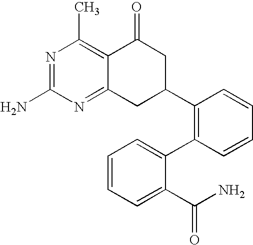 | C | 2′-(2-amino-4-methyl-5- oxo-5,6,7,8- tetrahydroquinazolin-7- yl)-1,1′-biphenyl-2- carboxamide |
| 91 | 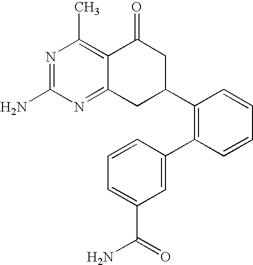 | B | 2′-(2-amino-4-methyl-5- oxo-5,6,7,8- tetrahydroquinazolin-7- yl)-1,1′-biphenyl-3- carboxamide |
| 92 | 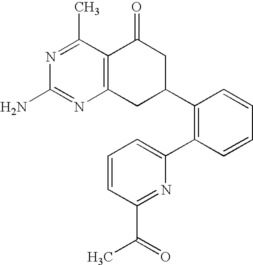 | B | 7-[2-(6-acetylpyridin-2- yl)phenyl]-2-amino-4- methyl-7,8- dihydroquinazolin-5(6H)- one |
| 93 | 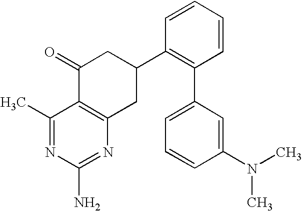 | A | 2-amino-7-[3′- (dimethylamino)-1,1′- biphenyl-2-yl]-4-methyl- 7,8-dihydroquinazolin- 5(6H)-one |
| 94 | 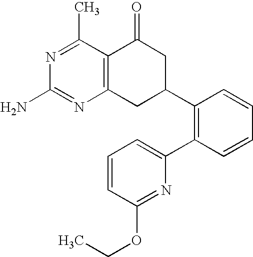 | B | 2-amino-7-[2-(6-ethoxy- pyridin-2-yl)-phenyl]-4- methyl-7,8-dihydro-6H- quinazolin-5-one |
| 95 | 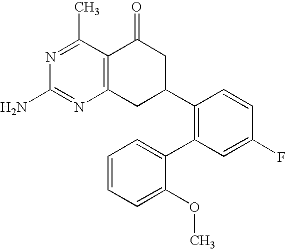 | C | 2-amino-7-(5-fluoro-2′- methoxy-biphenyl-2-yl)- 4-methyl-7,8-dihydro- 6H-quinazolin-5-one |
| 96 | 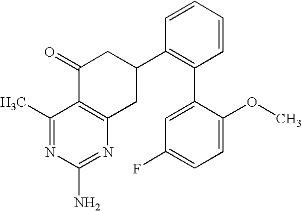 | C | 2-amino-7-(5′-fluoro-2′- methoxy-1,1′-biphenyl-2- yl)-4-methyl-7,8- dihydroquinazolin-5(6H)- one |
| 97 | 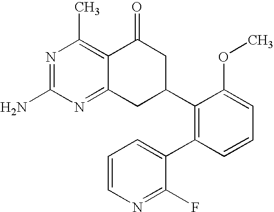 | C | 2-amino-7-[2-(2- fluoropyridin-3-yl)-6- methoxyphenyl]-4- methyl-7,8- dihydroquinazolin-5(6H)- one |
| 98 | 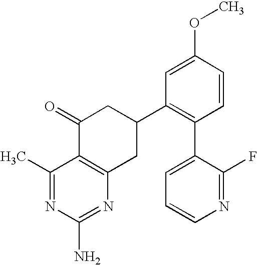 | C | 2-amino-7-[2-(2- fluoropyridin-3-yl)-5- methoxyphenyl]-4- methyl-7,8- dihydroquinazolin-5(6H)- one |
| 99 | 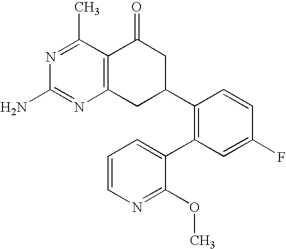 | C | 2-amino-7-[4-fluoro-2-(2- methoxy-pyridin-3-yl)- phenyl]-4-methyl-7,8- dihydro-6H-quinazolin-5- one |
| 100 | 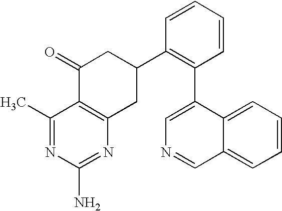 | B | 2-amino-7-(2-isoquinolin- 4-ylphenyl)-4-methyl- 7,8-dihydroquinazolin- 5(6H)-one |
| 101 | 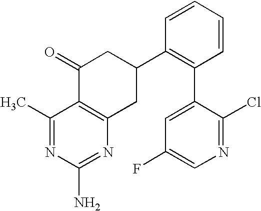 | C | 2-amino-7-[2-(2-chloro- 5-fluoropyridin-3- yl)phenyl]-4-methyl-7,8- dihydroquinazolin-5(6H)- one |
| 102 | 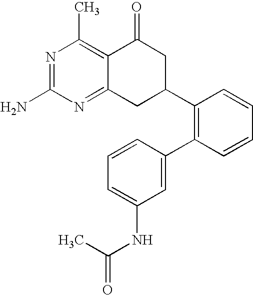 | B | N-[2′-(2-amino-4-methyl- 5-oxo-5,6,7,8- tetrahydroquinazolin-7- yl)-1,1′-biphenyl-3- yl]acetamide |
| 103 | 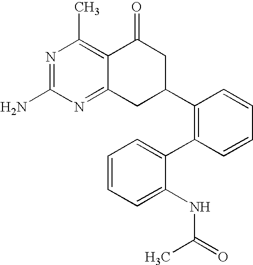 | B | N-[2′-(2-amino-4-methyl- 5-oxo-5,6,7,8- tetrahydroquinazolin-7- yl)-1,1′-biphenyl-2- yl]acetamide |
| 104 | 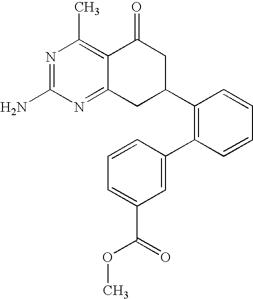 | A | methyl 2′-(2-amino-4- methyl-5-oxo-5,6,7,8- tetrahydroquinazolin-7- yl)-1,1′-biphenyl-3- carboxylate |
| 105 | 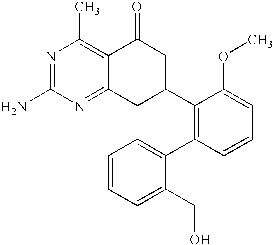 | C | 2-amino-7-[2′- (hydroxymethyl)-3- methoxy-1,1′-biphenyl-2- yl]-4-methyl-7,8- dihydroquinazolin-5(6H)- one |
| 106 | 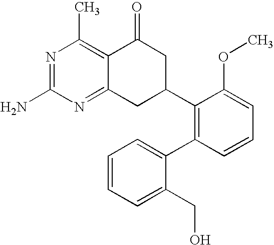 | C | 2-amino-7-[2′- (hydroxymethyl)-3- methoxy-1,1′-biphenyl-2- yl]-4-methyl-7,8- dihydroquinazolin-5(6H)- one |
| 107 | 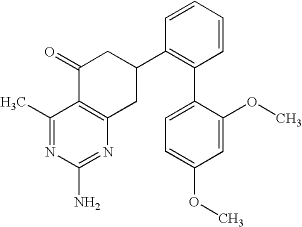 | B | 2-amino-7-(2′,4′- dimethoxy-1,1′-biphenyl- 2-yl)-4-methyl-7,8- dihydroquinazolin-5(6H)- one |
| 108 | 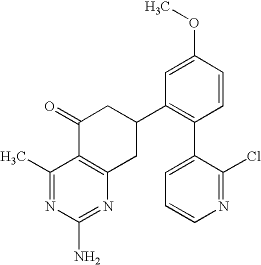 | C | 2-amino-7-[2-(2- chloropyridin-3-yl)-5- methoxyphenyl]-4- methyl-7,8- dihydroquinazolin-5(6H)- one |
| 109 | 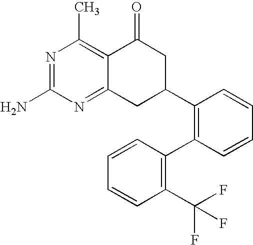 | C | 2-amino-4-methyl-7-[2′- (trifluoromethyl)-1,1′- biphenyl-2-yl]-7,8- dihydroquinazolin-5(6H)- one |
| 110 | 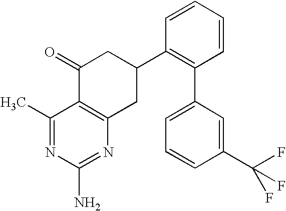 | B | 2-amino-4-methyl-7-[3′- (trifluoromethyl)-1,1′- biphenyl-2-yl]-7,8- dihydroquinazolin-5(6H)- one |
| 111 | 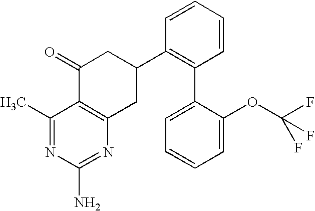 | C | 2-amino-4-methyl-7-[2′- (trifluoromethoxy)-1,1′- biphenyl-2-yl]-7,8- dihydroquinazolin-5(6H)- one |
| 112 | 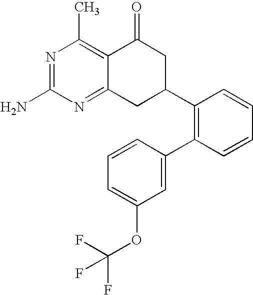 | A | 2-amino-4-methyl-7-[3′- (trifluoromethoxy)-1,1′- biphenyl-2-yl]-7,8- dihydroquinazolin-5(6H)- one |
| 113 | 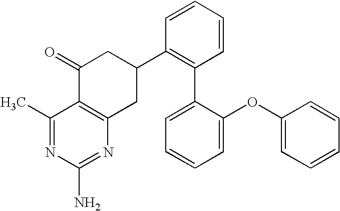 | C | 2-amino-4-methyl-7-(2′- phenoxy-1,1′-biphenyl-2- yl)-7,8- dihydroquinazolin-5(6H)- one |
| 114 | 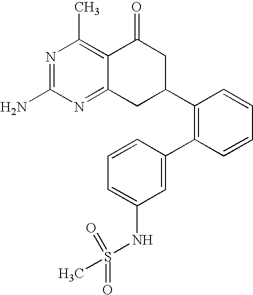 | B | N-[2′-(2-amino-4-methyl- 5-oxo-5,6,7,8- tetrahydroquinazolin-7- yl)-1,1′-biphenyl-3- yl]methanesulfonamide |
| 115 | 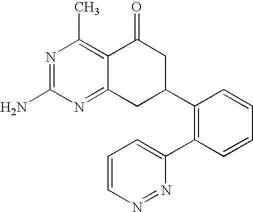 | B | 2-amino-4-methyl-7-(2- pyridazin-3-ylphenyl)- 7,8-dihydroquinazolin- 5(6H)-one |
| 116 | 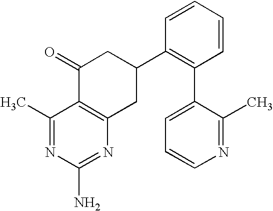 | C | 2-amino-4-methyl-7-[2- (2-methylpyridin-3- yl)phenyl]-7,8- dihydroquinazolin-5(6H)- one |
| 117 | 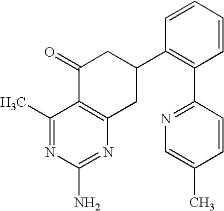 | C | 2-amino-4-methyl-7-[2- (5-methylpyridin-2- yl)phenyl]-7,8- dihydroquinazolin-5(6H)- one |
| 118 | 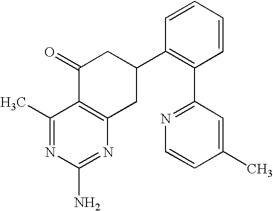 | C | 2-amino-4-methyl-7-[2- (4-methylpyridin-2- yl)phenyl]-7,8- dihydroquinazolin-5(6H)- one |
| 119 | 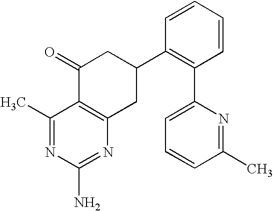 | A | 2-amino-4-methyl-7-[2- (6-methylpyridin-2- yl)phenyl]-7,8- dihydroquinazolin-5(6H)- one |
| 120 | 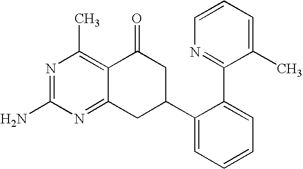 | B | 2-amino-4-methyl-7-[2- (3-methylpyridin-2- yl)phenyl]-7,8- dihydroquinazolin-5(6H)- one |
| 121 | 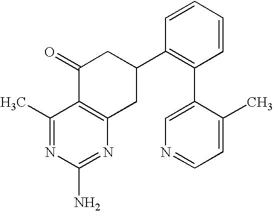 | A | 2-amino-4-methyl-7-[2- (4-methylpyridin-3- yl)phenyl]-7,8- dihydroquinazolin-5(6H)- one |
| 122 | 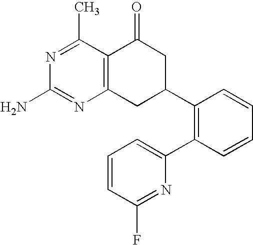 | C | 2-amino-7-[2-(6- fluoropyridin-2- yl)phenyl]-4-methyl-7,8- dihydroquinazolin-5(6H)- one |
| 123 | 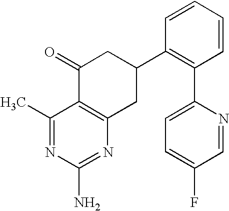 | C | 2-amino-7-[2-(5- fluoropyridin-2- yl)phenyl]-4-methyl-7,8- dihydroquinazolin-5(6H)- one |
| 124 | 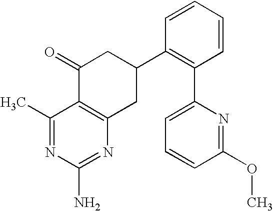 | C | 2-amino-7-[2-(6- methoxypyridin-2- yl)phenyl]-4-methyl-7,8- dihydroquinazolin-5(6H)- one |
| 125 | 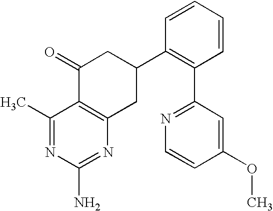 | C | 2-amino-7-[2-(4- methoxy-pyridin-2-yl)- phenyl]-4-methyl-7,8- dihydro-6H-quinazolin-5- one |
| 126 | 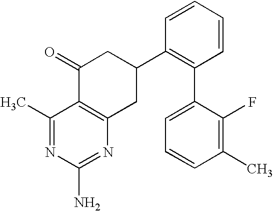 | C | 2-amino-7-(2′-fluoro-3′- methyl-1,1′-biphenyl-2- yl)-4-methyl-7,8- dihydroquinazolin-5(6H)- one |
| 127 | 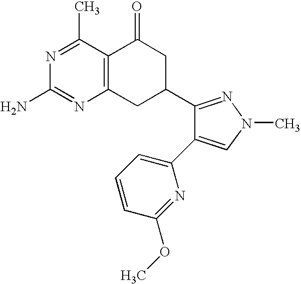 | C | 2-amino-7-[4-(6- methoxy-pyridin-2-yl)-1- methyl-1H-pyrazol-3-yl]- 4-methyl-7,8-dihydro- 6H-quinazolin-5-one |
| 128 | 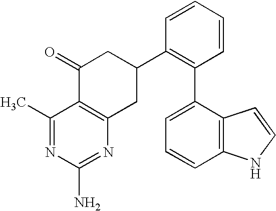 | C | 2-amino-7-[2-(1H-indol- 4-yl)phenyl]-4-methyl- 7,8-dihydroquinazolin- 5(6H)-one |
| 129 | 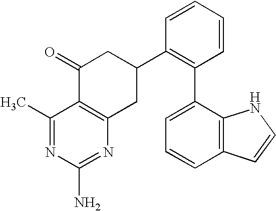 | C | 2-amino-7-[2-(1H-indol- 7-yl)phenyl]-4-methyl- 7,8-dihydroquinazolin- 5(6H)-one |
| 130 | 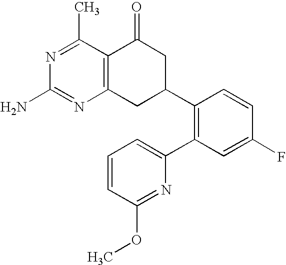 | C | 2-amino-7-[4-fluoro-2-(6- methoxy-pyridin-2-yl)- phenyl]-4-methyl-7,8- dihydro-6H-quinazolin-5- one |
| 131 | 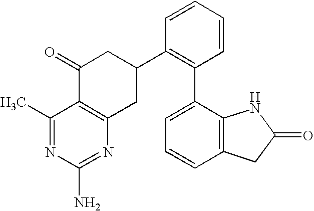 | B | 2-amino-4-methyl-7-[2- (2-oxo-2,3-dihydro-1H- indol-7-yl)phenyl]-7,8- dihydroquinazolin-5(6H)- one |
| 132 | 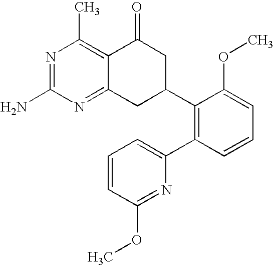 | C | 2-amino-7-[2-methoxy-6- (6-methoxy-pyridin-2-yl)- phenyl]-4-methyl-7,8- dihydro-6H-quinazolin-5- one |
| 133 | 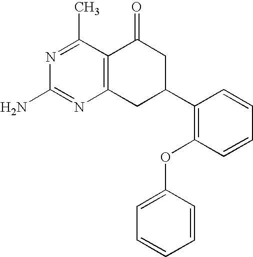 | C | 2-amino-4-methyl-7-(2- phenoxyphenyl)-7,8- dihydroquinazolin-5(6H)- one |
| 134 | 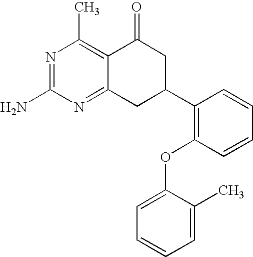 | C | 2-amino-4-methyl-7-[2- (2- methylphenoxy)phenyl]- 7,8-dihydroquinazolin- 5(6H)-one |
| 135 | 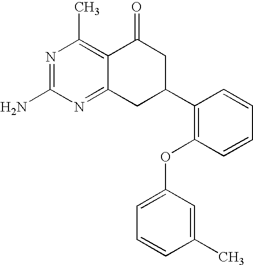 | C | 2-amino-4-methyl-7-[2- (3- methylphenoxy)phenyl]- 7,8-dihydroquinazolin- 5(6H)-one |
| 136 | 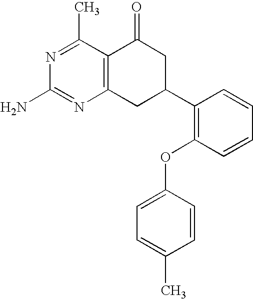 | A | 2-amino-4-methyl-7-[2- (4- methylphenoxy)phenyl]- 7,8-dihydroquinazolin- 5(6H)-one |
| 137 | 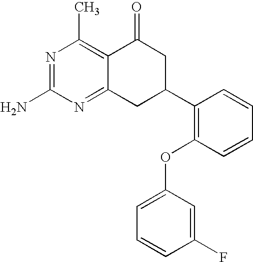 | C | 2-amino-7-[2-(3- fluorophenoxy)phenyl]-4- methyl-7,8- dihydroquinazolin-5(6H)- one |
| 138 | 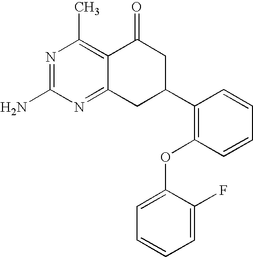 | C | 2-amino-7-[2-(2- fluorophenoxy)phenyl]-4- methyl-7,8- dihydroquinazolin-5(6H)- one |
| 139 | 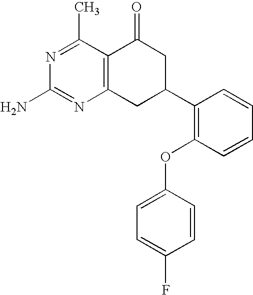 | B | 2-amino-7-[2-(4- fluorophenoxy)phenyl]-4- methyl-7,8- dihydroquinazolin-5(6H)- one |
| 140 | 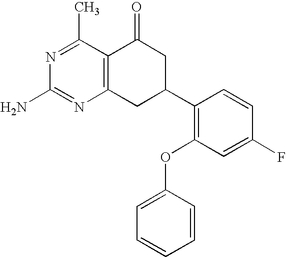 | C | 2-amino-7-(4-fluoro-2- phenoxy-phenyl)-4- methyl-7,8-dihydro-6H- quinazolin-5-one |
| 141 | 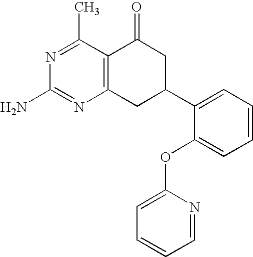 | C | 2-amino-4-methyl-7-[2- (pyridin-2-yloxy)phenyl]- 7,8-dihydroquinazolin- 5(6H)-one |
| 142 | 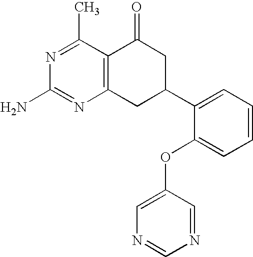 | C | 2-amino-4-methyl-7-[2- (pyrimidin-5- yloxy)phenyl]-7,8- dihydroquinazolin-5(6H)- one |
| 143 | 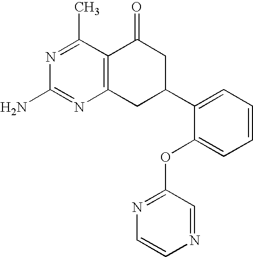 | C | 2-amino-4-methyl-7-[2- (pyrazin-2-yloxy)phenyl]- 7,8-dihydroquinazolin- 5(6H)-one |
| 144 | 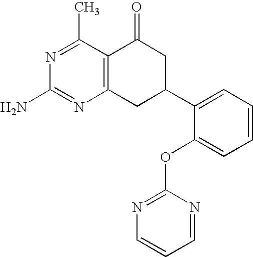 | B | 2-amino-4-methyl-7-[2- (pyrimidin-2- yloxy)phenyl]-7,8- dihydroquinazolin-5(6H)- one |
| 145 | 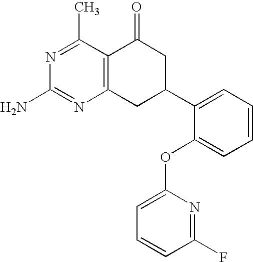 | C | 2-amino-7-{2-[(6- fluoropyridin-2- yl)oxy]phenyl}-4-methyl- 7,8-dihydroquinazolin- 5(6H)-one |
| 146 | 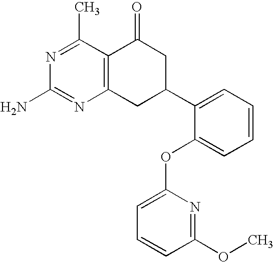 | C | 2-amino-7-{2-[(6- methoxypyridin-2- yl)oxy]phenyl}-4-methyl- 7,8-dihydroquinazolin- 5(6H)-one |
| 147 | 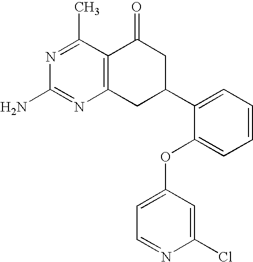 | B | 2-amino-7-{2-[(2- chloropyridin-4- yl)oxy]phenyl}-4-methyl- 7,8-dihydroquinazolin- 5(6H)-one |
| 148 | 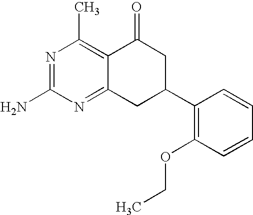 | C | 2-amino-7-(2-ethoxy- phenyl)-4-methyl-7,8- dihydro-6H-quinazolin-5- one |
| 149 | 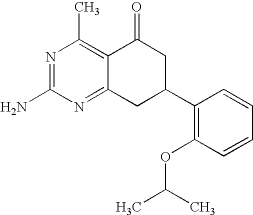 | C | 2-amino-7-(2-isopropoxy- phenyl)-4-methyl-7,8- dihydro-6H-quinazolin-5- one |
| 150 | 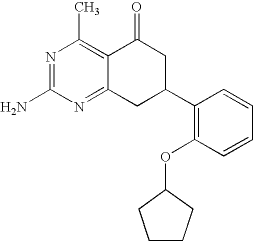 | C | 2-amino-7-(2- cyclopentyloxy-phenyl)- 4-methyl-7,8-dihydro- 6H-quinazolin-5-one |
| 151 | 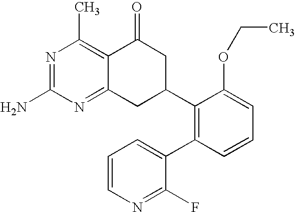 | C | 2-amino-7-[2-ethoxy-6- (2-fluoro-pyridin-3-yl)- phenyl]-4-methyl-7,8- dihydro-6H-quinazolin-5- one |
| 152 | 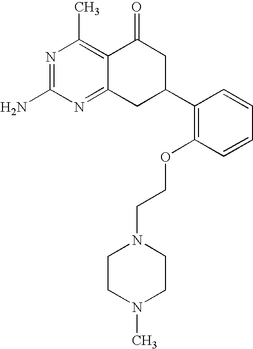 | B | 2-amino-4-methyl-7-{2- [2-(4-methyl-piperazin-1- yl)-ethoxy]-phenyl}-7,8- dihydro-6H-quinazolin-5- one |
| 153 | 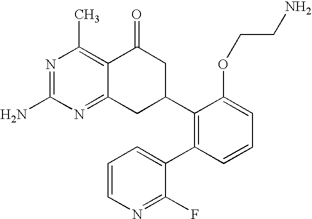 | C | 2-amino-7-[2-(2-amino- ethoxy)-6-(2-fluoro- pyridin-3-yl)-phenyl]-4- methyl-7,8-dihydro-6H- quinazolin-5-one |
| 154 | 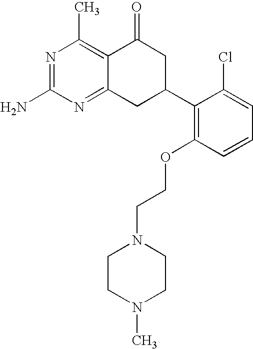 | A | 2-amino-7-{2-chloro-6- [2-(4-methyl-piperazin-1- yl)-ethoxy]-phenyl}-4- methyl-7,8-dihydro-6H- quinazolin-5-one |
| 155 | 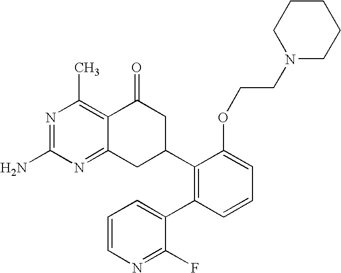 | C | 2-amino-7-[2-(2-fluoro- pyridin-3-yl)-6-(2- piperidin-1-yl-ethoxy)- phenyl]-4-methyl-7,8- dihydro-6H-quinazolin-5- one |
| 156 | 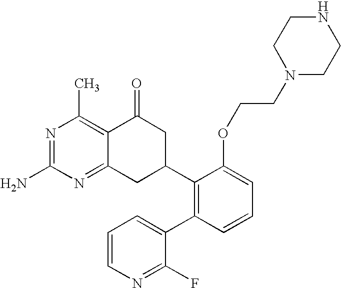 | C | 2-amino-7-[2-(2-fluoro- pyridin-3-yl)-6-(2- piperazin-1-yl-ethoxy)- phenyl]-4-methyl-7,8- dihydro-6H-quinazolin-5- one |
| 157 | 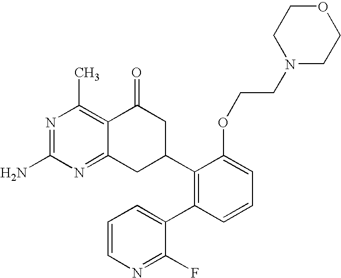 | C | 2-amino-7-[2-(2-fluoro- pyridin-3-yl)-6-(2- morpholin-4-yl-ethoxy)- phenyl]-4-methyl-7,8- dihydro-6H-quinazolin-5- one |
| 158 | 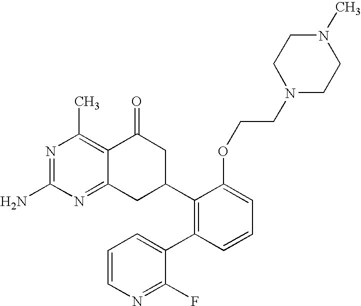 | C | 2-amino-7-{2-(2-fluoro- pyridin-3-yl)-6-[2-(4- methyl-piperazin-1-yl)- ethoxy]-phenyl}-4- methyl-7,8-dihydro-6H- quinazolin-5-one |
| 159 | 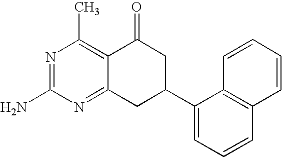 | B | 2-amino-4-methyl-7-(1- naphthyl)-7,8- dihydroquinazolin-5(6H)- one |
| 160 | 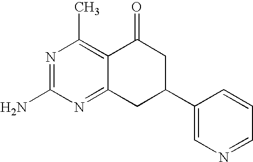 | A | 2-amino-4-methyl-7- pyridin-3-yl-7,8- dihydroquinazolin-5(6H)- one |
| 161 | 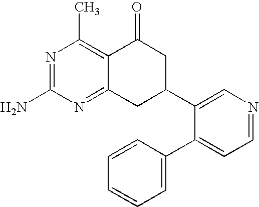 | C | 2-amino-4-methyl-7-(4- phenylpyridin-3-yl)-7,8- dihydroquinazolin-5(6H)- one |
| 162 | 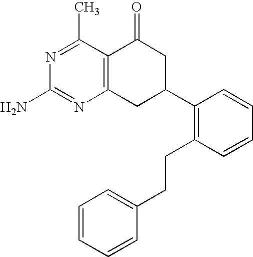 | A | 2-amino-4-methyl-7-[2- (2-phenylethyl)phenyl]- 7,8-dihydroquinazolin- 5(6H)-one |
| 163 | 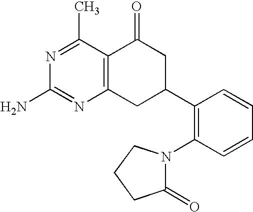 | B | 2-amino-4-methyl-7-[2- (2-oxo-pyrrolidin-1-yl)- phenyl]-7,8-dihydro-6H- quinazolin-5-one |
| 164 | 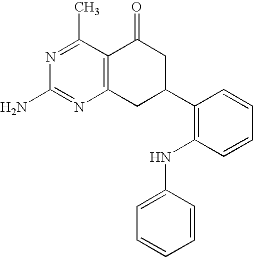 | C | 2-amino-4-methyl-7-(2- phenylamino-phenyl)- 7,8-dihydro-6H- quinazolin-5-one |
| 165 | 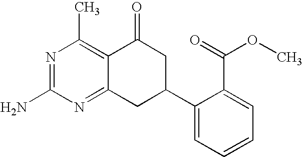 | C | methyl 2-(2-amino-4- methyl-5-oxo-5,6,7,8- tetrahydroquinazolin-7- yl)benzoate |
| 166 | 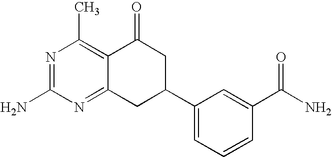 | A | 3-(2-amino-4-methyl-5- oxo-5,6,7,8- tetrahydroquinazolin-7- yl)benzamide |
| 167 | 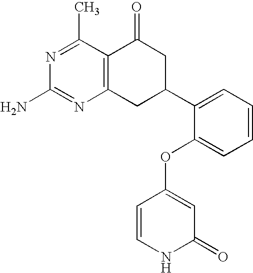 | B | 2-amino-4-methyl-7-[2- (2-oxo-1,2-dihydro- pyridin-4-yloxy)-phenyl]- 7,8-dihydro-6H- quinazolin-5-one |
| 168 | 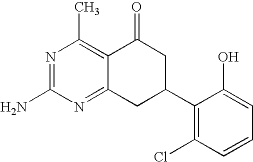 | A | 2-Amino-7-(2-chloro-6- hydroxy-phenyl)-4- methyl-7,8-dihydro-6H- quinazolin-5-one |
| 169 | 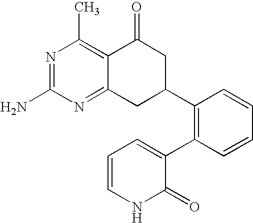 | C | 2-amino-4-methyl-7-[2- (2-oxo-1,2- dihydropyridin-3- yl)phenyl]-7,8- dihydroquinazolin-5(6H)- one |
| 170 | 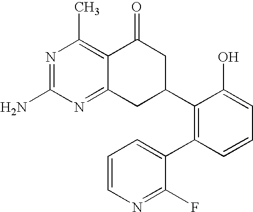 | C | 2-amino-7-[2-(2-fluoro- pyridin-3-yl)-6-hydroxy- phenyl]-4-methyl-7,8- dihydro-6H-quinazolin-5- one |
| 171 | 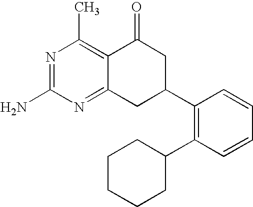 | C | 2-amino-7-(2- cyclohexylphenyl)-4- methyl-7,8- dihydroquinazolin-5(6H)- one |
| 172 | 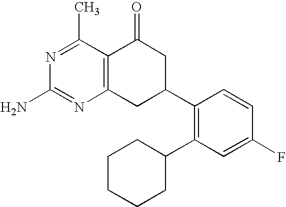 | B | 2-amino-7-(2-cyclohexyl- 4-fluorophenyl)-4- methyl-7,8- dihydroquinazolin-5(6H)- one |
| 173 | 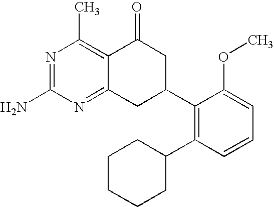 | A | 2-amino-7-(2-cyclohexyl- 6-methoxyphenyl)-4- methyl-7,8- dihydroquinazolin-5(6H)- one |
| 174 | 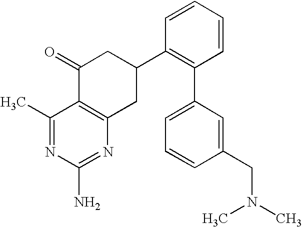 | B | 2-amino-7-{3′- [(dimethylamino)methyl]- 1,1′-biphenyl-2-yl}-4- methyl-7,8- dihydroquinazolin-5(6H)- one |

TABLE Ia

| | Synthesis | | LCMS m/z | LCMS | | |
|---|---|---|---|---|---|---|
| Cmpd. | Method | MW | observed | Rt | Intermediate A | Intermediate B |
| 1 | A | 219.29 | 220.1 | 1.69 | 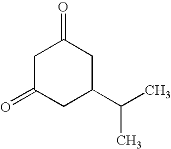 | |
| 2 | A | 283.33 | 284.3 | 1.868 | 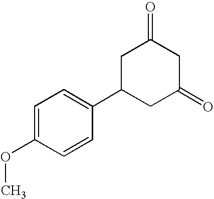 | |
| 3 | A | 287.75 | 288.7 | 2.139 | 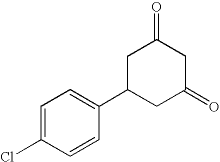 | |
| 4 | A | 288.74 | 289 | 2.44 | 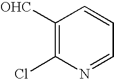 | |
| 5 | A | 317.77 | 318 | 2.37 | 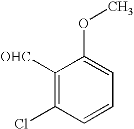 | |
| 6 | A | 322.19 | 324.0 (M + 2) | 2.384 | 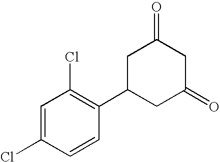 | |
| 7 | A | 332.20 | 334 | 2.38 | 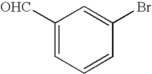 | |
| 8 | A | 332.20 | 332 | 2.38 | 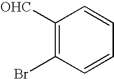 | |
| 9 | A | 333.39 | 334.2 | 1.51 | | |
| 10 | A | 336.19 | 336.0 | 1.90 | | |
| 11 | A | 336.19 | 336.0 | 1.90 | | |
| 12 | A | 336.44 | 337 | 1.89 | 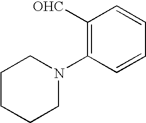 | |
| 13 | A | 338.41 | 339 | 2.13 | 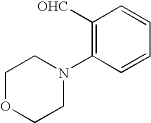 | |
| 14 | A | 343.43 | 344 | 3.4 | 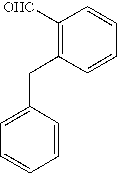 | |
| 15 | A | 343.43 | 344.3 | 2.55 | 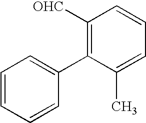 | |
| 16 | A | 350.19 | 350.0 | 2.42 | 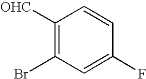 | |
| 17 | A | 351.45 | 352 | 2.86 | 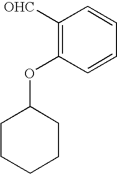 | |
| 18 | A | 362.23 | 363 | 2.38 | 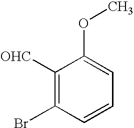 | |
| 19 | A | 362.41 | 363.3 | 2.16 | 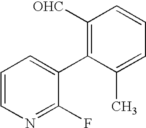 | |
| 20 | A | 365.47 | 366 | 2.67 | 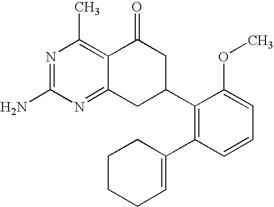 | |
| 21 | A | 366.44 | 367 | 2.38 | | |
| 22 | A | 375.43 | 376 | 2.68 | 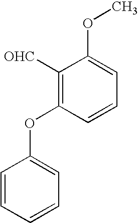 | |
| 23 | A | 393.47 | 394 | 3.64 | | |
| 24 | A | 346.39 | 347.3 | 1.57 | 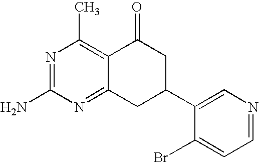 | 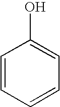 |
| 25 | B | 330.39 | 331 | 1.67 | 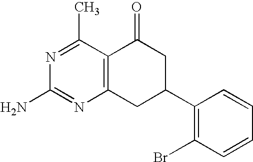 | 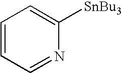 |
| 26 | B | 330.39 | 331 | 1.66 | 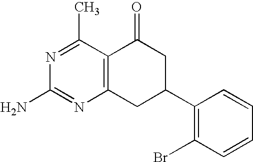 | 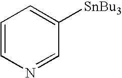 |
| 27 | B | 330.39 | 331 | 1.66 | 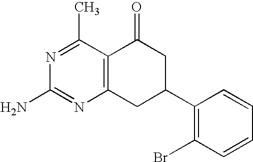 | 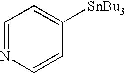 |
| 28 | B | 330.39 | 331.1 | 1.63 | 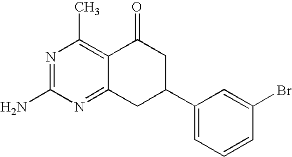 | 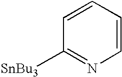 |
| 29 | B | 330.39 | 331.1 | 1.65 | 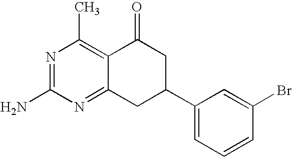 | 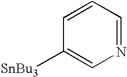 |
| 30 | B | 330.39 | 331.1 | 1.63 | 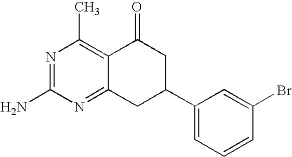 | 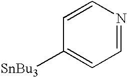 |
| 31 | B | 331.38 | 332 | 2.88 | 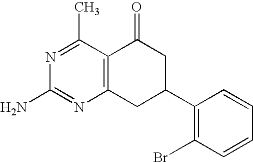 | 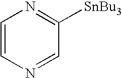 |
| 32 | B | 331.38 | 332 | 1.89 | 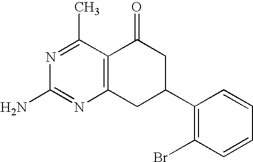 | 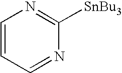 |
| 33 | B | 331.38 | 332 | 1.64 | 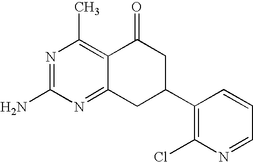 | 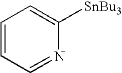 |
| 34 | B | 332.37 | 333 | 1.46 | 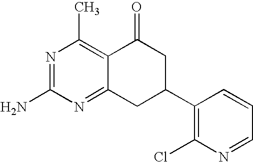 | 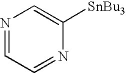 |
| 35 | B | 336.42 | 337 | 3.44 | 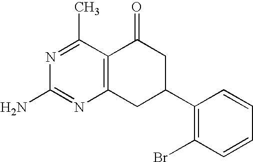 | 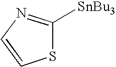 |
| 36 | B | 337.41 | 338 | 2.01 | 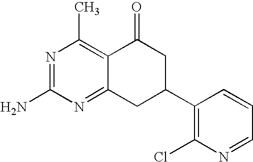 | 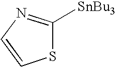 |
| 37 | B | 353.42 | 354 | 2.88 | 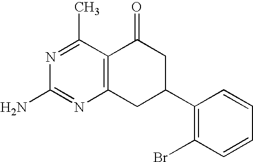 | 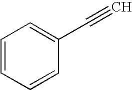 |
| 38 | B | 407.48 | 408 | 1.63 | 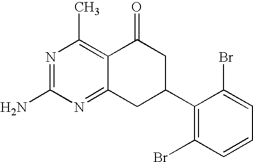 | 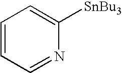 |
| 39 | C | 319.37 | 320 | 1.92 | 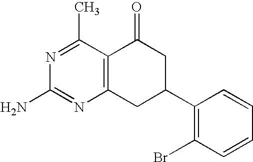 | 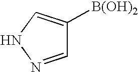 |
| 40 | C | 319.41 | 320 | 2.73 | 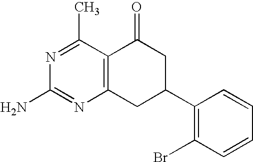 | 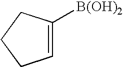 |
| 41 | C | 329.40 | 330 | 2.66 | 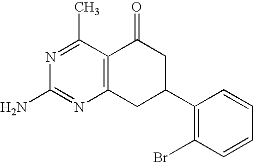 | 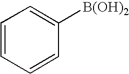 |
| 42 | C | 331.38 | 332.2 | 1.71 | 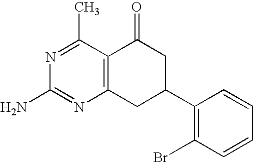 | 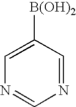 |
| 43 | C | 333.39 | 334.2 | 1.87 | 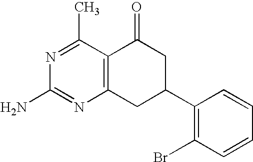 | 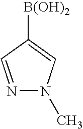 |
| 44 | C | 333.43 | 334 | 2.86 | 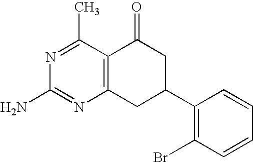 | 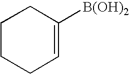 |
| 45 | C | 334.42 | 335 | 1.61 | 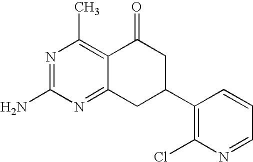 | 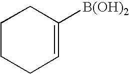 |
| 46 | C | 335.43 | 336 | 4.27 | 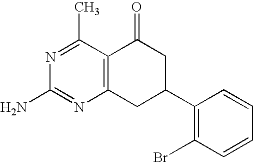 | 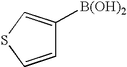 |
| 47 | C | 339.44 | 340.2 | 2.42 | 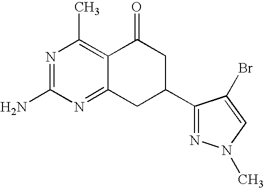 | |
| 48 | C | 339.44 | 340.1 | 2.26 | | |
| 49 | C | 343.43 | 344.2 | 2.55 | 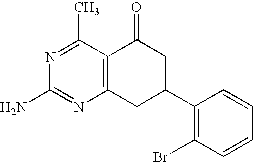 | 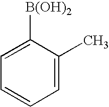 |
| 50 | C | 343.43 | 344.2 | 2.58 | 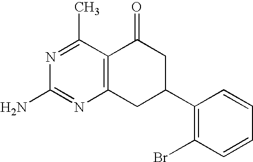 | 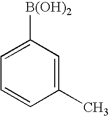 |
| 51 | C | 347.39 | 348.2 | 2.46 | 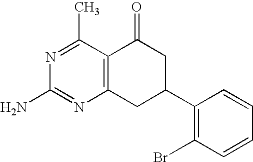 | 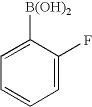 |
| 52 | C | 347.39 | 348.2 | 2.47 | 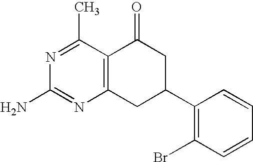 | 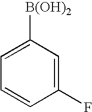 |
| 53 | C | 347.39 | 348.2 | 2.52 | 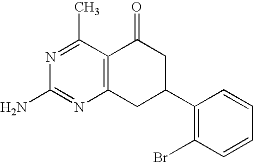 | 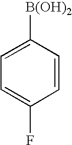 |
| 54 | C | 347.39 | 349.1 | 2.24 | 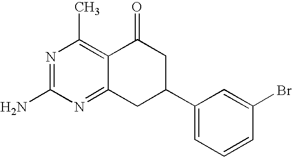 | 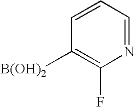 |
| 55 | C | 348.38 | 349.3 | 2.09 | 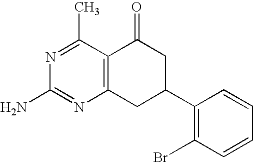 | 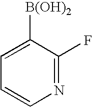 |
| 56 | C | 348.38 | 349.3 | 2.11 | 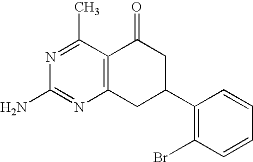 | 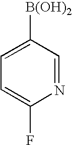 |
| 57 | C | 348.38 | 349 | 1.43 | 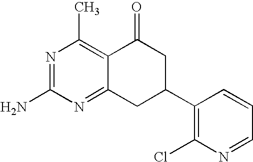 | 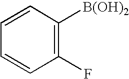 |
| 58 | C | 348.38 | 348.1 | 2.64 | 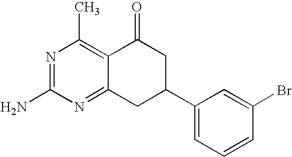 | 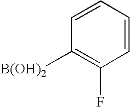 |
| 59 | C | 348.40 | 349.2 | 2.08 | 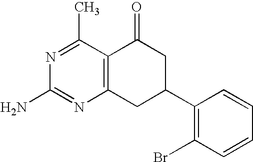 | 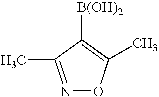 |
| 60 | C | 349.37 | 350.1 | 1.97 | 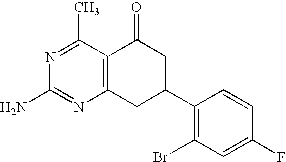 | 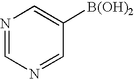 |
| 61 | C | 349.37 | 350.3 | 1.25 | 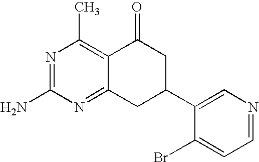 | 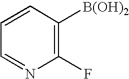 |
| 62 | C | 349.43 | 350 | 2.61 | 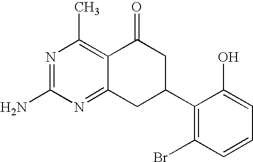 | 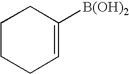 |
| 63 | C | 352.37 | 353.0 | 1.80 | | |
| 64 | C | 352.37 | 353.1 | 1.73 | | |
| 65 | C | 354.41 | 355.3 | 2.31 | 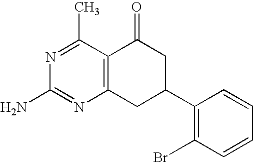 | 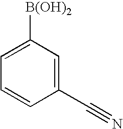 |
| 66 | C | 358.44 | 359.3 | 1.59 | 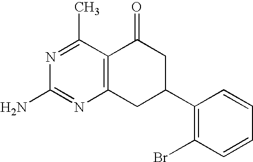 | 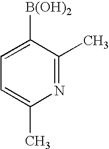 |
| 67 | C | 359.43 | | | 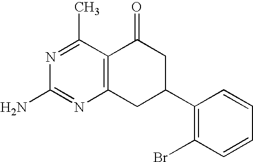 | 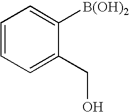 |
| 68 | C | 359.43 | | | 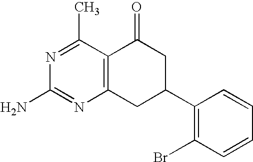 | 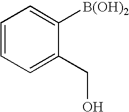 |
| 69 | C | 359.43 | 360 | 2.65 | 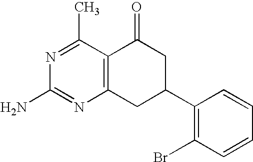 | 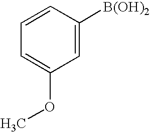 |
| 70 | C | 360.42 | 361.4 | 2.16 | 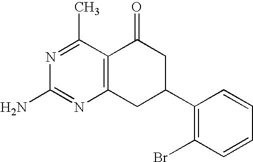 | 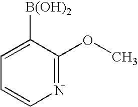 |
| 71 | C | 360.42 | 361.3 | 1.61 | 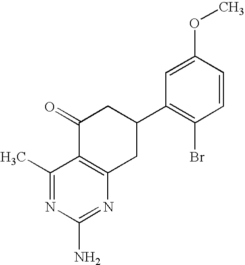 | 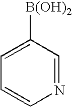 |
| 72 | C | 360.42 | 361.2 | 1.63 | 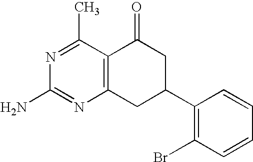 | 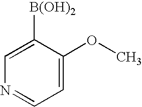 |
| 73 | C | 360.42 | 361.2 | 2.69 | 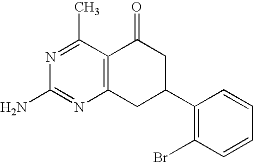 | 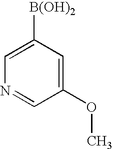 |
| 74 | C | 360.42 | 361.3 | 2.12 | 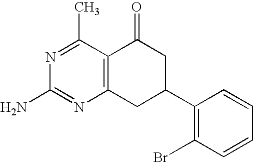 | 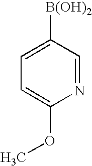 |
| 75 | C | 361.40 | 362.2 | 1.78 | 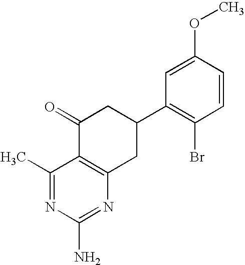 | 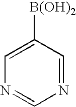 |
| 76 | C | 361.42 | 362.3 | 2.61 | 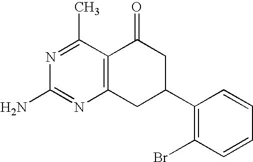 | 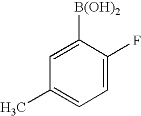 |
| 77 | C | 361.42 | 362.3 | 2.62 | 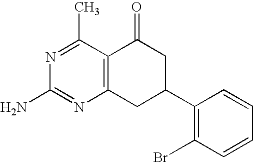 | 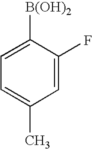 |
| 78 | C | 363.46 | 364 | 2.86 | 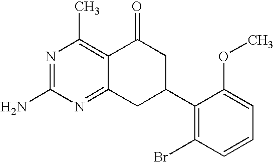 | 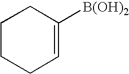 |
| 79 | C | 363.85 | 364 | 2.68 | 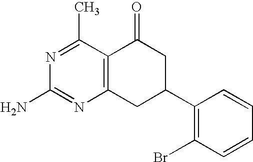 | 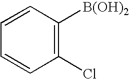 |
| 80 | C | 364.83 | 365.2 | 2.08 | 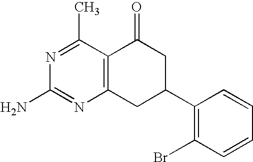 | 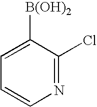 |
| 81 | C | 364.83 | 365.2 | 2.19 | 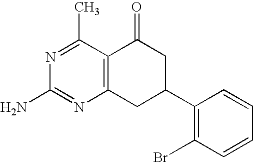 | 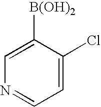 |
| 82 | C | 364.83 | 365.2 | 2.17 | 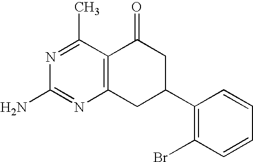 | 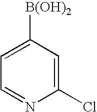 |
| 83 | C | 365.38 | 366.3 | 2.46 | 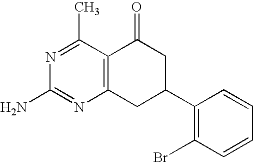 | 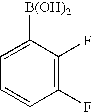 |
| 84 | C | 365.38 | 366.2 | 2.47 | 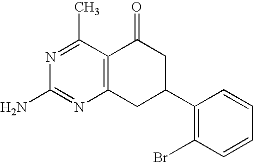 | 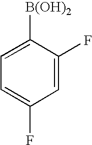 |
| 85 | C | 365.38 | 366.2 | 2.15 | 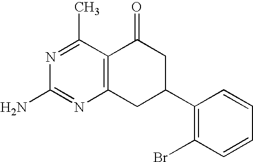 | 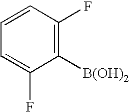 |
| 86 | C | 365.38 | 367.1 | 2.27 | 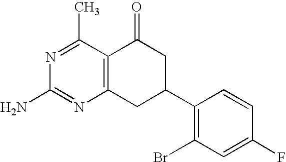 | 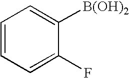 |
| 87 | C | 366.37 | 367.1 | 2.27 | 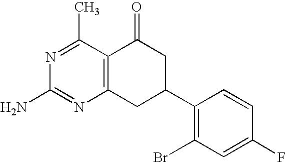 | 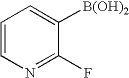 |
| 88 | C | 366.37 | 367.3 | 2.12 | 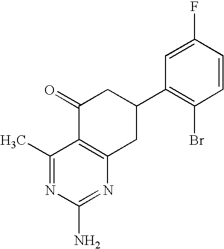 | 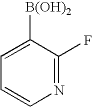 |
| 89 | C | 366.37 | 367.1 | 2.42 | 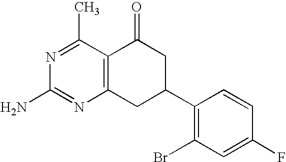 | 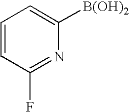 |
| 90 | C | 372.43 | 373 | 2.07 | 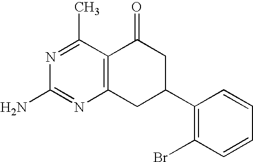 | 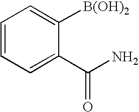 |
| 91 | C | 372.43 | 373 | 2.11 | 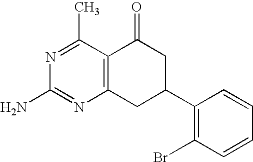 | 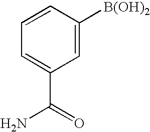 |
| 92 | C | 372.43 | 395.2 (+N a) | 2.18 | 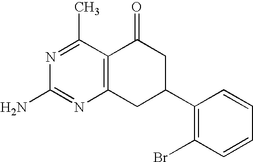 | 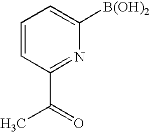 |
| 93 | C | 372.47 | 373.3 | 1.79 | 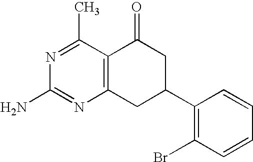 | 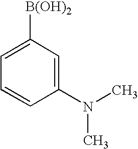 |
| 94 | C | 374.44 | 375.3 | 2.36 | 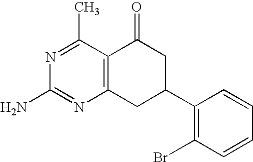 | 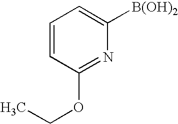 |
| 95 | C | 377.42 | 378.1 | 2.60 | 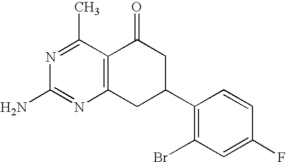 | 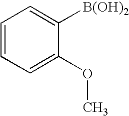 |
| 96 | C | 377.42 | 378.3 | 2.44 | 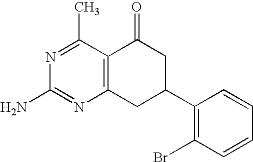 | 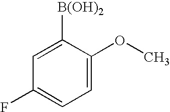 |
| 97 | C | 378.41 | 379 | 2.28 | 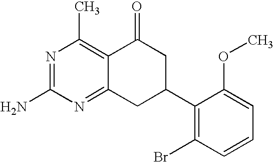 | 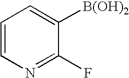 |
| 98 | C | 378.41 | 379.3 | 2.09 | 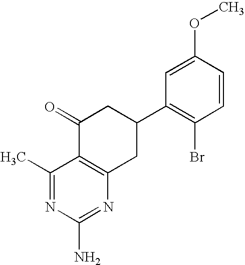 | 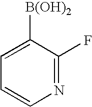 |
| 99 | C | 378.41 | 379.1 | 2.37 | 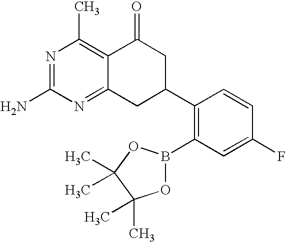 | 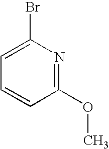 |
| 100 | C | 380.45 | 381.3 | 1.77 | 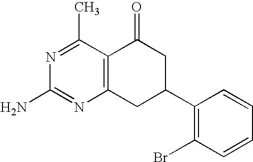 | 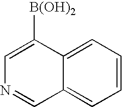 |
| 101 | C | 382.82 | 383.2 | 2.23 | 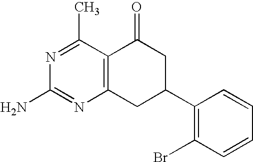 | 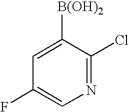 |
| 102 | C | 386.45 | 387 | 2.27 | 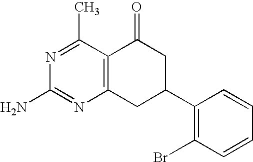 | 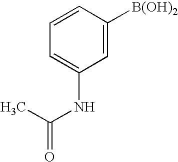 |
| 103 | C | 386.45 | 387 | 2.18 | 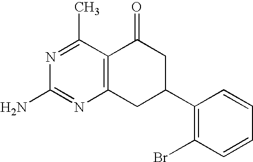 | 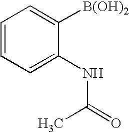 |
| 104 | C | 387.44 | 388 | 2.62 | 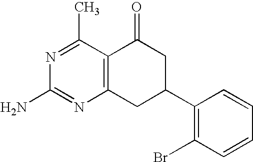 | 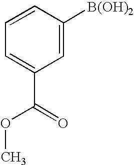 |
| 105 | C | 389.45 | 390 | 2.17 | 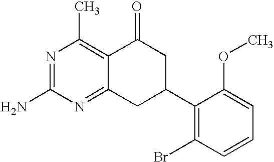 | 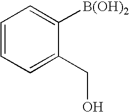 |
| 106 | C | 389.45 | 390 | 2.31 | 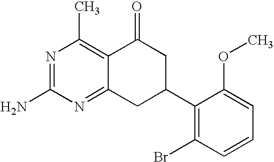 | 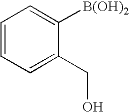 |
| 107 | C | 389.45 | 390.3 | 2.42 | 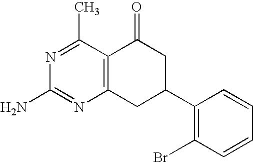 | 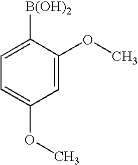 |
| 108 | C | 394.86 | 395.2 | 2.12 | 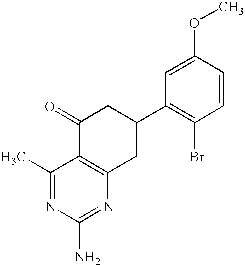 | 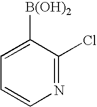 |
| 109 | C | 397.40 | 398 | 2.8 | 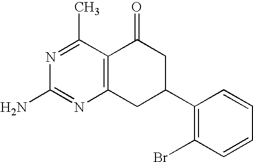 | 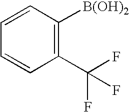 |
| 110 | C | 397.40 | 398.3 | 2.69 | 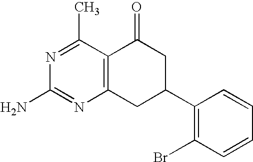 | 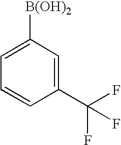 |
| 111 | C | 413.40 | 414.4 | 2.66 | 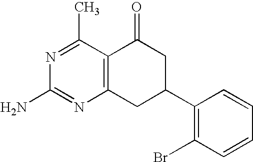 | 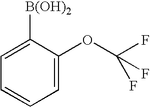 |
| 112 | C | 413.40 | 414 | 2.97 | 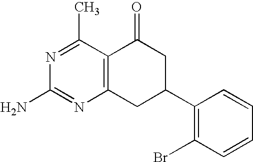 | 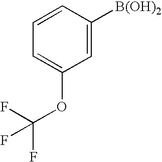 |
| 113 | C | 421.50 | 422.3 | 2.78 | 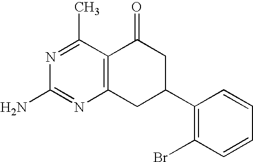 | 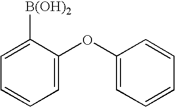 |
| 114 | C | 422.51 | 423 | 2.33 | 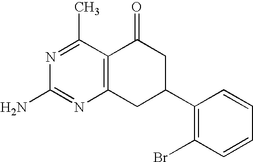 | 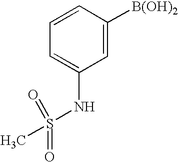 |
| 115 | D | 331.38 | 332 | 1.82 | 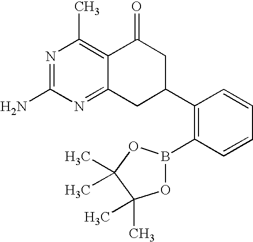 | 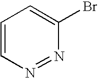 |
| 116 | D | 344.42 | 345.3 | 1.57 | 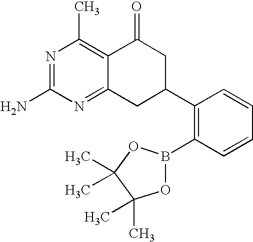 | 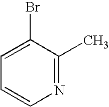 |
| 117 | D | 344.42 | 345.2 | 1.5 | 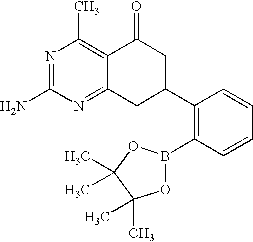 | 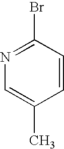 |
| 118 | D | 344.42 | 345.2 | 1.5 | 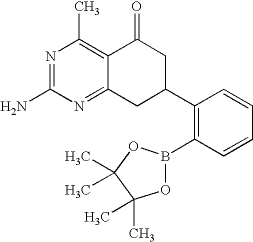 | 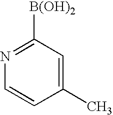 |
| 119 | D | 344.42 | 345.3 | 1.45 | 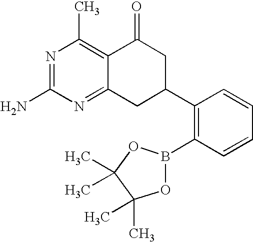 | 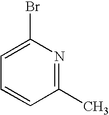 |
| 120 | D | 344.42 | 345.4 | 1.51 min | 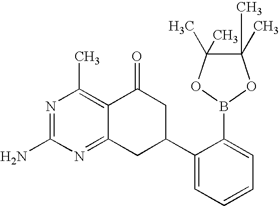 | 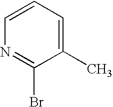 |
| 121 | D | 344.42 | 345.3 | 1.58/1.67 | 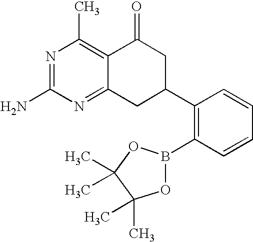 | 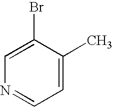 |
| 122 | D | 348.38 | 349.2 | 2.11 | 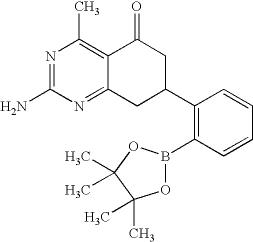 | 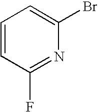 |
| 123 | D | 348.38 | 349.3 | 2 | 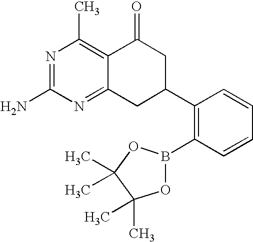 | 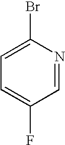 |
| 124 | D | 360.42 | 361.3 | 2.26 | 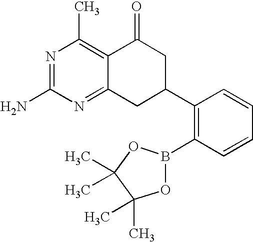 | 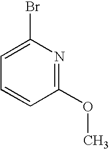 |
| 125 | D | 360.42 | 361.2 | 1.72 | 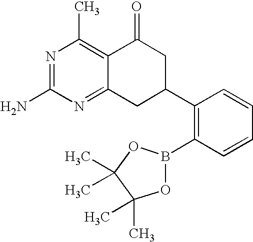 | 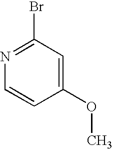 |
| 126 | D | 361.42 | 362.3 | 2.55 | 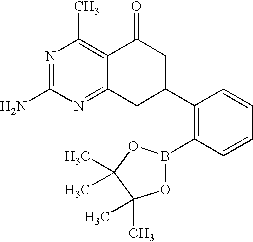 | 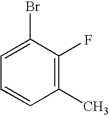 |
| 127 | D | 364.41 | 365.1 | 2.13 | 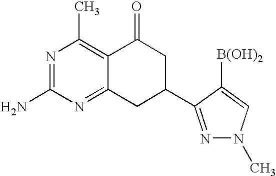 | |
| 128 | D | 368.44 | 369.3 | 2.32 | 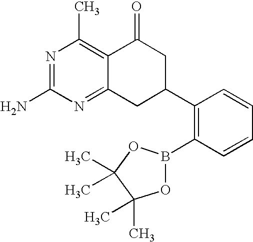 | 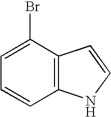 |
| 129 | D | 368.44 | 369.4 | 2.39 | 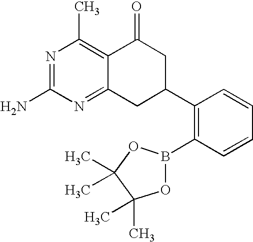 | 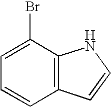 |
| 130 | D | 378.41 | 379 | 2.54 | 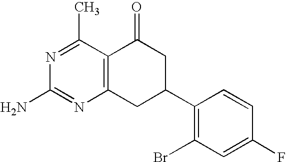 | 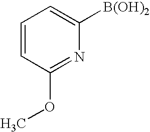 |
| 131 | D | 384.44 | 385.4 | 1.92/2.09 | 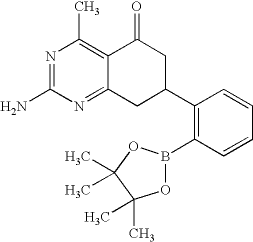 | 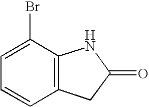 |
| 132 | D | 390.44 | 391 | 3.05 | 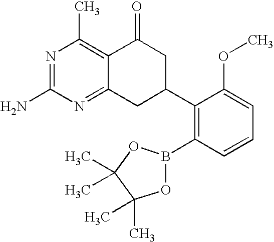 | 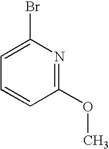 |
| 133 | E | 345.40 | 346 | 2.55 | 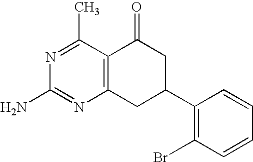 | 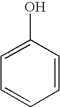 |
| 134 | E | 359.43 | 360 | 2.83 | 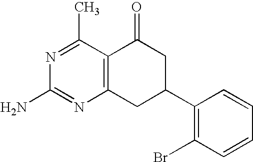 | 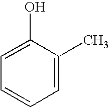 |
| 135 | E | 359.43 | 360 | 2.84 | 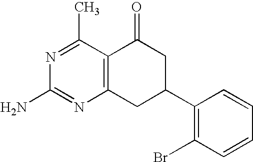 | 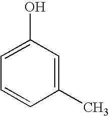 |
| 136 | E | 359.43 | 360 | 2.86 | 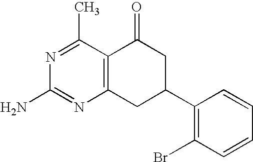 | 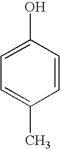 |
| 137 | E | 363.39 | 364 | 2.71 | 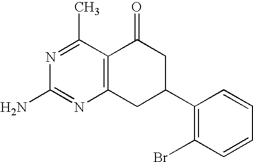 | 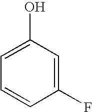 |
| 138 | E | 363.39 | 364 | 2.65 | 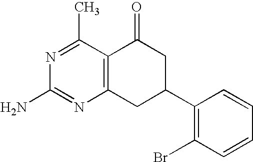 | 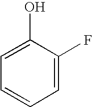 |
| 139 | E | 363.39 | 364 | 2.72 | 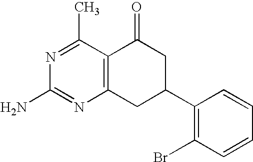 | 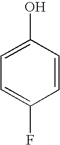 |
| 140 | E | 363.39 | 364.1 | 2.73 | 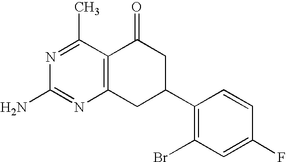 | 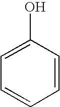 |
| 141 | F | 346.39 | 347 | 2.16 | 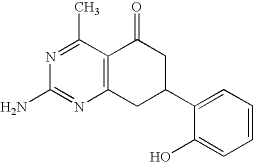 | 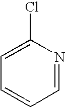 |
| 142 | F | 347.38 | 348 | 1.96 | 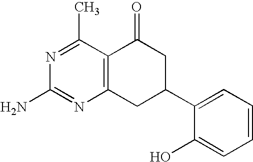 | 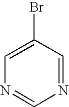 |
| 143 | F | 347.38 | 348 | 2.03 | 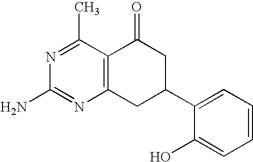 | 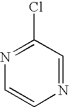 |
| 144 | F | 347.38 | 348 | 1.92 | 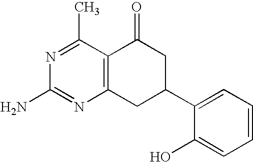 | 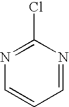 |
| 145 | F | 364.38 | 365 | 2.42 | 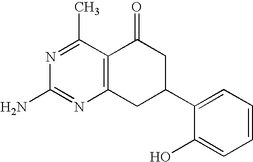 | 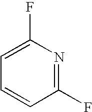 |
| 146 | F | 376.41 | 377 | 2.54 | 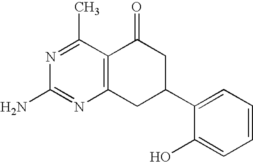 | 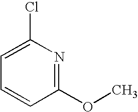 |
| 147 | F | 380.83 | 381 | 3.84 | 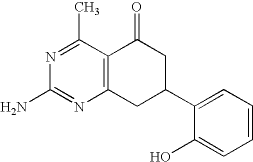 | 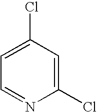 |
| 148 | G | 297.36 | 298 | 2.9 | 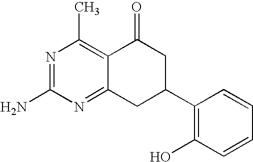 | |
| 149 | G | 311.38 | 312 | 3.01 | 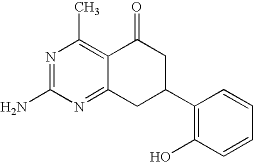 | 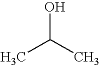 |
| 150 | G | 337.42 | 338 | 3.46 | 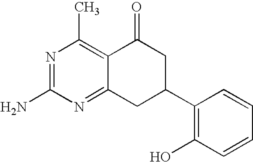 | 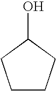 |
| 151 | G | 392.43 | 393 | 2.47 | 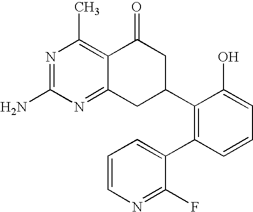 | |
| 152 | G | 395.50 | 396 | 2.37 | 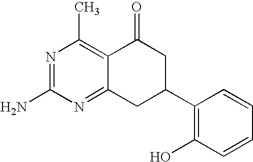 | |
| 153 | G | 407.45 | 408 | 2.72 | 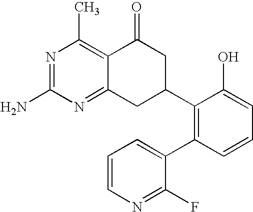 | |
| 154 | G | 429.95 | 430 | 2.98 | | |
| 155 | G | 475.57 | 476 | 2.93 | 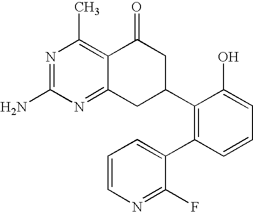 | |
| 156 | G | 476.55 | 477 | 2.75 | 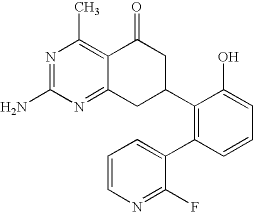 | |
| 157 | G | 477.54 | 478 | 2.8 | 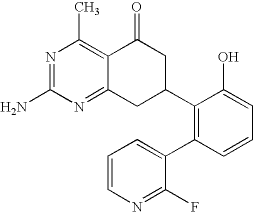 | |
| 158 | G | 490.58 | 477 | 2.83 | 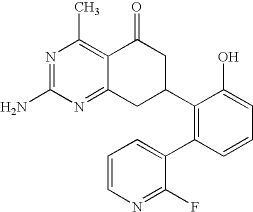 | |
| 159 | H | 303.36 | 304.3 | 1.57 | 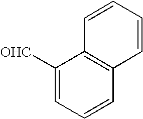 | |
| 160 | I | 254.29 | 255 | 0.75 | 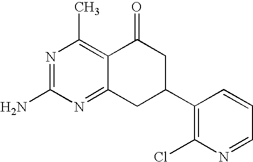 | |
| 161 | I | 330.39 | 331.2 | 1.6 | 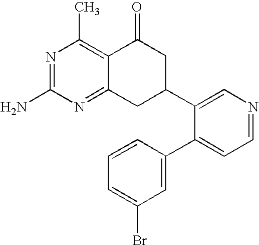 | |
| 162 | J | 357.46 | 358 | 2.83 | 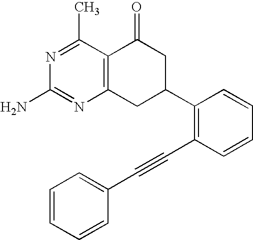 | |
| 163 | L | 336.39 | 337 | 1.79 | 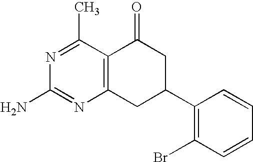 | 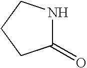 |
| 164 | M | 344.42 | 345 | 2.61 | 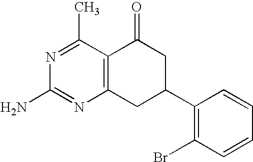 | 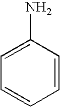 |
| 165 | N | 311.34 | | | 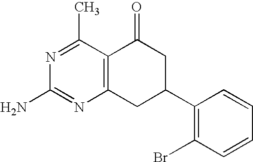 | |
| 166 | O | 296.33 | | | 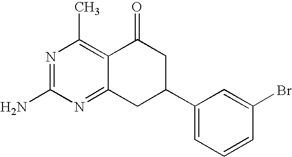 | |
| 167 | P | 362.39 | 363 | 1.87 | 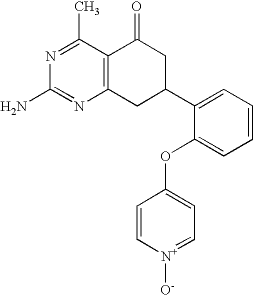 | |
| 168 | Q | 303.75 | 304 | 2.11 | 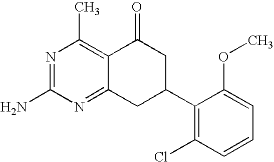 | |
| 169 | Q | 346.39 | 347 | 2.66 | 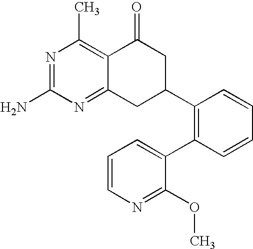 | |
| 170 | Q | 364.38 | 365 | 3.22 | 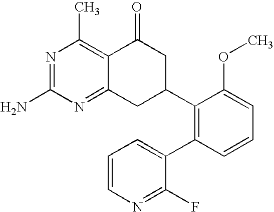 | |
| 171 | R | 335.45 | 336 | 2.95 | 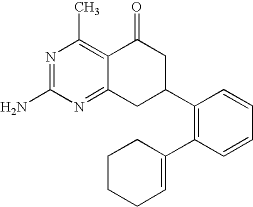 | |
| 172 | R | 353.44 | 354.1 | 2.64 | 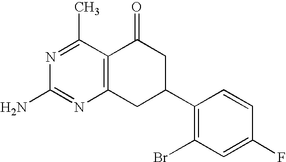 | |
| 173 | R | 365.47 | 366 | 2.62 | 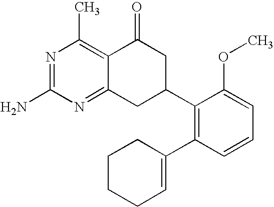 | |
| 174 | S | 386.50 | 387.3 | 1.8 | 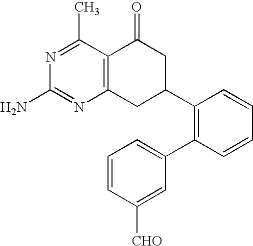 | |

The compounds in Table II were prepared in a similar manner to the compounds and procedures described above (compound 312 was not synthesized).

TABLE II

| | | | | Hsp90 |
|---|---|---|---|---|
| | | | | IC50 |
| | | | | Range |
| | | | Rt (min.); | A = >10 μM, |
| | | | m/z | B = 1-10 μM, |
| Compound | Structure | Name | observed | C = <1 μM |
| 175 | 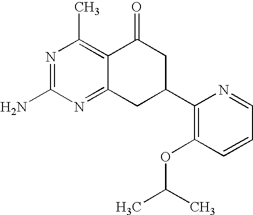 | 2-amino-7-(3- isopropoxy-pyridin- 2-yl)-4-methyl-7,8- dihydro-6H- quinazolin-5-one | Rt = 1.74 m/z = 313.2 | C |
| 176 | 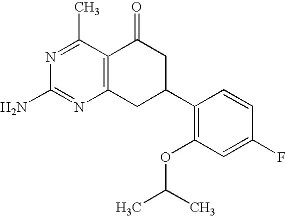 | 2-amino-7-(4-fluoro- 2-isopropoxy- phenyl)-4-methyl- 7,8-dihydro-6H- quinazolin-5-one | Rt = 4.29 m/z = 330.2 | C |
| 177 | 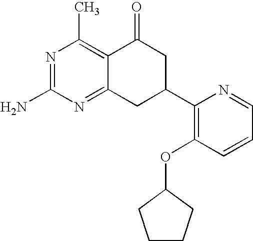 | 2-amino-7-(3- cyclopentyloxy- pyridin-2-yl)-4- methyl-7,8-dihydro- 6H-quinazolin-5-one | Rt = 2.07 m/z = 339.2 | C |
| 178 | 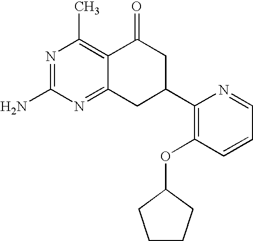 | 2-amino-7-(3- cyclopentyloxy- pyridin-2-yl)-4- methyl-7,8-dihydro- 6H-quinazolin-5-one | Rt = 2.07 m/z = 339.2 | C |
| 179 | 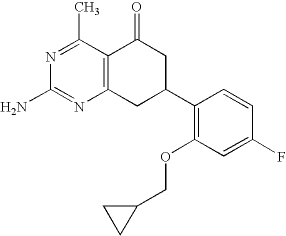 | 2-amino-7-(2- cyclopropylmethoxy- 4-fluoro-phenyl)-4- methyl-7,8-dihydro- 6H-quinazolin-5-one | Rt = 4.51 m/z = 342.2 | C |
| 180 | 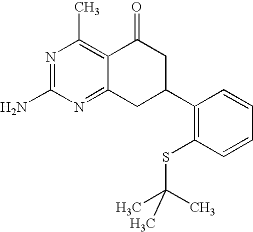 | 2-amino-7-(2-tert- butylsulfanyl- phenyl)-4-methyl- 7,8-dihydro-6H- quinazolin-5-one | Rt = 3.56 m/z = 342.2 | C |
| 181 | 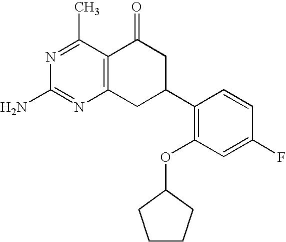 | 2-amino-7-(2- cyclopentyloxy-4- fluoro-phenyl)-4- methyl-7,8-dihydro- 6H-quinazolin-5-one | Rt = 4.86 m/z = 356.2 | C |
| 182 | 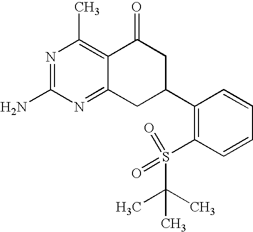 | 2-amino-4-methyl-7- [2-(2-methyl- propane-2-sulfonyl)- phenyl]-7,8-dihydro- 6H-quinazolin-5-one | Rt = 2.47 m/z = 374.1 | C |
| 183 | 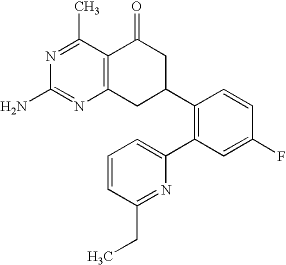 | 2-amino-7-[2-(6- ethyl-pyridin-2-yl)- 4-fluoro-phenyl]-4- methyl-7,8-dihydro- 6H-quinazolin-5-one | Rt = 1.679 m/z = 377.2 | C |
| 184 | 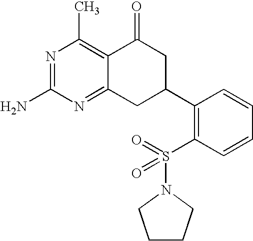 | 2-amino-4-methyl-7- [2-(pyrrolidine-1- sulfonyl)-phenyl]- 7,8-dihydro-6H- quinazolin-5-one | Rt = 2.18 m/z = 386 | B |
| 185 | 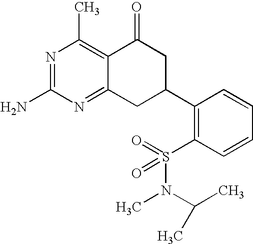 | 2-(2-amino-4- methyl-5-oxo- 5,6,7,8-tetrahydro- quinazolin-7-yl)-N- isopropyl-N-methyl- benzenesulfonamide | Rt = 2.31 m/z = 388 | B |
| 186 | 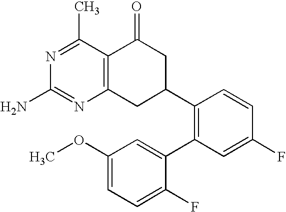 | 2-amino-7-(5,2′- difluoro-5′-methoxy- biphenyl-2-yl)-4- methyl-7,8-dihydro- 6H-quinazolin-5-one | Rt = 2.485 m/z = 396.3 | C |
| 187 | 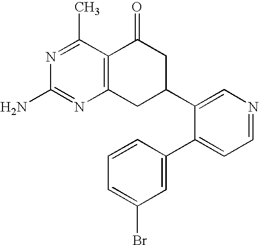 | 2-amino-7-[4-(3- bromo-phenyl)- pyridin-3-yl]-4- methyl-7,8-dihydro- 6H-quinazolin-5-one | Rt = 1.93 m/z = 411.0 | C |
| 188 | 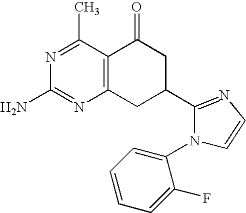 | 2-amino-7-[1-(2- fluoro-phenyl)-1H- imidazol-2-yl]-4- methyl-7,8-dihydro- 6H-quinazolin-5-one | Rt = 1.67 m/z = 338 | C |
| 189 | 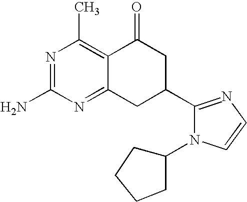 | 2-amino-7-(1- cyclopentyl-1H- imidazol-2-yl)-4- methyl-7,8-dihydro- 6H-quinazolin-5-one | Rt = 1.64 m/z = 312 | C |
| 190 | 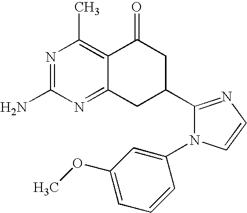 | 2-amino-7-[1-(3- methoxy-phenyl)- 1H-imidazol-2-yl]-4- methyl-7,8-dihydro- 6H-quinazolin-5-one | Rt = 1.76 m/z = 350 | B |
| 191 | 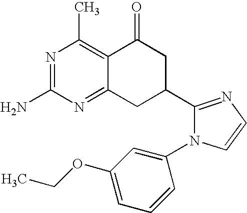 | 2-amino-7-[1-(3- ethoxy-phenyl)-1H- imidazol-2-yl]-4- methyl-7,8-dihydro- 6H-quinazolin-5-one | Rt = 1.92 m/z = 364 | A |
| 192 | 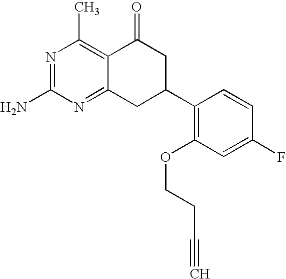 | 2-amino-7-(2-but-3- ynyloxy-4-fluoro- phenyl)-4-methyl- 7,8-dihydro-6H- quinazolin-5-one | Rt = 2.50 m/z = 340 | C |
| 193 | 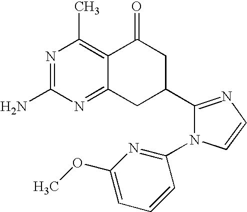 | 2-amino-7-[1-(6- methoxy-pyridin-2- yl)-1H-imidazol-2- yl]-4-methyl-7,8- dihydro-6H- quinazolin-5-one | Rt = 1.76 m/z = 351 | C |
| 194 | 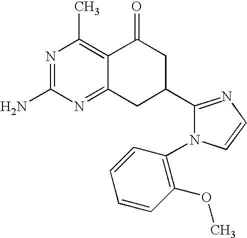 | 2-amino-7-[1-(2- methoxy-phenyl)- 1H-imidazol-2-yl]-4- methyl-7,8-dihydro- 6H-quinazolin-5-one | Rt = 1.74 m/z = 350 | C |
| 195 | 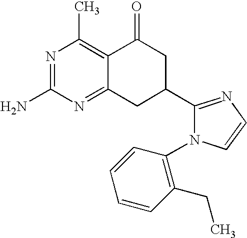 | 2-amino-7-[1-(2- ethyl-phenyl)-1H- imidazol-2-yl]-4- methyl-7,8-dihydro- 6H-quinazolin-5-one | Rt = 1.92 m/z = 348 | C |
| 196 | 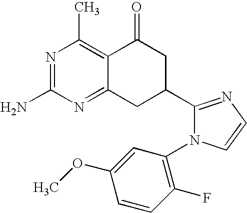 | 2-amino-7-[1-(2- fluoro-5-methoxy- phenyl)-1H- imidazol-2-yl]-4- methyl-7,8-dihydro- 6H-quinazolin-5-one | Rt = 1.79 m/z = 368 | B |
| 197 | 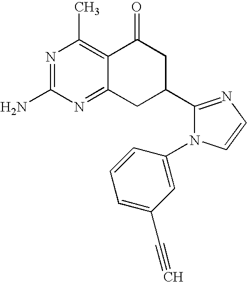 | 2-amino-7-[1-(3- ethynyl-phenyl)-1H- imidazol-2-yl]-4- methyl-7,8-dihydro- 6H-quinazolin-5-one | Rt = 1.81 m/z = 344 | C |
| 198 | 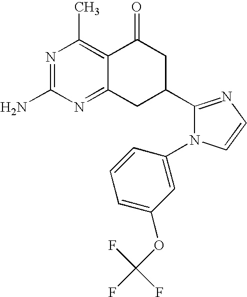 | 2-amino-4-methyl-7- [1-(3- trifluoromethoxy- phenyl)-1H- imidazol-2-yl]-7,8- dihydro-6H- quinazolin-5-one | Rt = 2.03 m/z = 404 | A |
| 199 | 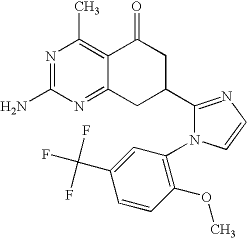 | 2-amino-7-[1-(2- methoxy-5- trifluoromethyl- phenyl)-1H- imidazol-2-yl]-4- methyl-7,8-dihydro- 6H-quinazolin-5-one | Rt = 2.03 m/z = 418 | B |
| 200 | 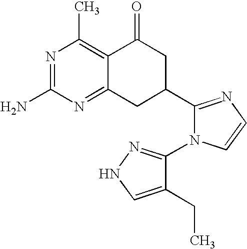 | 2-amino-7-[1-(4- ethyl-1H-pyrazol-3- yl)-1H-imidazol-2- yl]-4-methyl-7,8- dihydro-6H- quinazolin-5-one | Rt = 1.57 m/z = 338 | C |
| 201 | 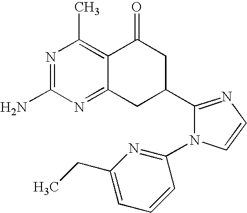 | 2-amino-7-[1-(6- ethyl-pyridin-2-yl)- 1H-imidazol-2-yl]-4- methyl-7,8-dihydro- 6H-quinazolin-5-one | Rt = 1.81 m/z = 349 | B |
| 202 | 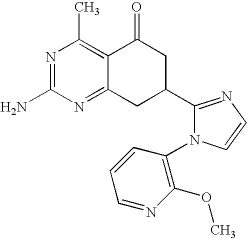 | 2-amino-7-[1-(2- methoxy-pyridin-3- yl)-1H-imidazol-2- yl]-4-methyl-7,8- dihydro-6H- quinazolin-5-one | Rt = 1.66 m/z = 351 | C |
| 203 | 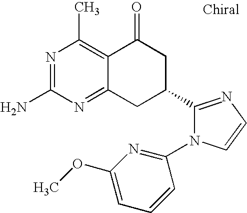 | (R)-2-amino-7-[1-(6- methoxy-pyridin-2- yl)-1H-imidazol-2- yl]-4-methyl-7,8- dihydro-6H- quinazolin-5-one | Rt = 1.66 m/z = 351 | B |
| 204 | 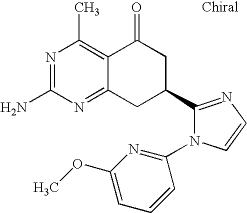 | (S)-2-amino-7-[1-(6- methoxy-pyridin-2- yl)-1H-imidazol-2- yl]-4-methyl-7,8- dihydro-6H- quinazolin-5-one | Rt = 1.66 m/z = 351 | C |
| 205 | 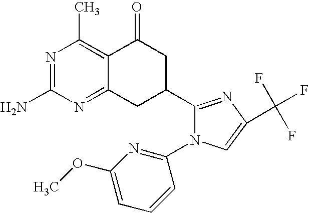 | 2-amino-7-[1-(6- methoxy-pyridin-2- yl)-4-trifluoromethyl- 1H-imidazol-2- yl]-4-methyl-7,8- dihydro-6H- quinazolin-5-one | Rt = 2.37 m/z = 419 | C |
| 206 | 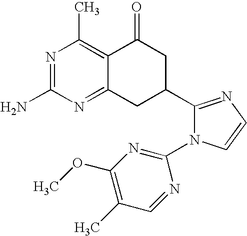 | 2-amino-7-[1-(4- methoxy-5-methyl- pyrimidin-2-yl)-1H- imidazol-2-yl]-4- methyl-7,8-dihydro- 6H-quinazolin-5-one | Rt = 1.83 m/z = 366 | C |
| 207 | 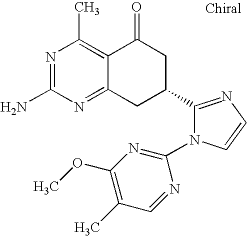 | (R)-2-amino-7-[1-(4- methoxy-5-methyl- pyrimidin-2-yl)-1H- imidazol-2-yl]-4- methyl-7,8-dihydro- 6H-quinazolin-5-one | Rt = 1.83 m/z = 366 | A |
| 208 | 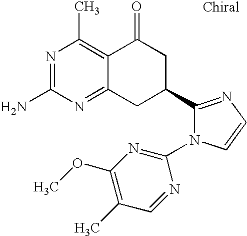 | (S)-2-amino-7-[1-(4- methoxy-5-methyl- pyrimidin-2-yl)-1H- imidazol-2-yl]-4- methyl-7,8-dihydro- 6H-quinazolin-5-one | Rt = 1.83 m/z = 366 | C |
| 209 | 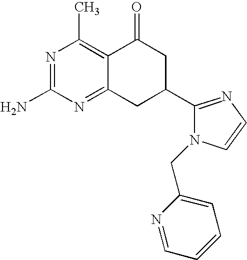 | 2-amino-4-methyl-7- (1-pyridin-2- ylmethyl-1H- imidazol-2-yl)-7,8- dihydro-6H- quinazolin-5-one | Rt = 1.36 m/z = 335 | A |
| 210 | 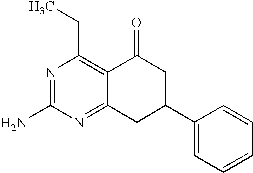 | 2-amino-4-ethyl-7- phenyl-7,8-dihydro- 6H-quinazolin-5-one | Rt = 2.11 m/z = 268 | A |
| 211 | 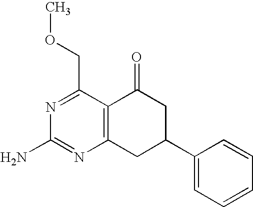 | 2-amino-4- methoxymethyl-7- phenyl-7,8-dihydro- 6H-quinazolin-5-one | | A |
| 212 | 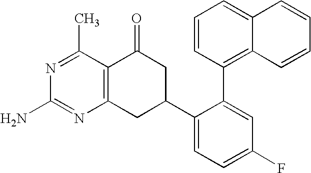 | 2-amino-7-(4-fluoro- 2-naphthalen-1-yl- phenyl)-4-methyl- 7,8-dihydro-6H- quinazolin-5-one | Rt = 2.4 m/z = 398.5 | C |
| 213 | 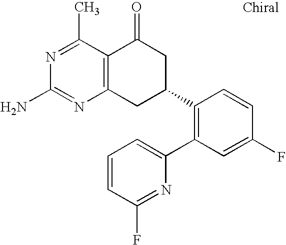 | (R)-2-amino-7-[4- fluoro-2-(6-fluoro- pyridin-2-yl)- phenyl]-4-methyl- 7,8-dihydro-6H- quinazolin-5-one | Rt = 2.15 m/z = 367 | B |
| 214 | 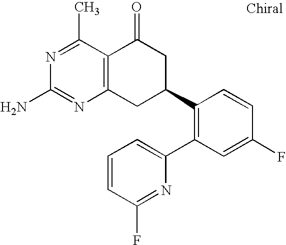 | (S)-2-amino-7-[4- fluoro-2-(6-fluoro- pyridin-2-yl)- phenyl]-4-methyl- 7,8-dihydro-6H- quinazolin-5-one | Rt = 2.15 m/z = 367 | C |
| 215 | 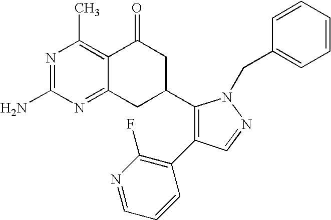 | 2-amino-7-[2- benzyl-4-(2-fluoro- pyridin-3-yl)-2H- pyrazol-3-yl]-4- methyl-7,8-dihydro- 6H-quinazolin-5-one | Rt = 2.08 m/z = 429 | B |
| 216 | 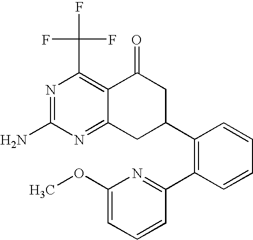 | 2-amino-7-[2-(6- methoxy-pyridin-2- yl)-phenyl]-4- trifluoromethyl-7,8- dihydro-6H- quinazolin-5-one | Rt = 3.99 m/z = 414 | C |
| 217 | 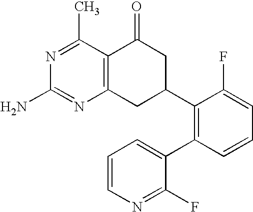 | 2-amino-7-[2-fluoro- 6-(2-fluoro-pyridin- 3-yl)-phenyl]-4- methyl-7,8-dihydro- 6H-quinazolin-5-one | Rt = 2.044 m/z = 367.3 | C |
| 218 | 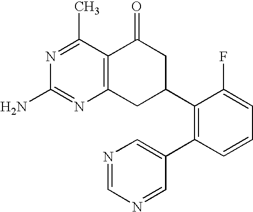 | 2-amino-7-(2-fluoro- 6-pyrimidin-5-yl- phenyl)-4-methyl- 7,8-dihydro-6H- quinazolin-5-one | Rt = 1.726 m/z = 350.3 | C |
| 219 | 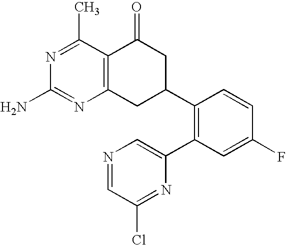 | 2-amino-7-[2-(6- chloro-pyrazin-2-yl)- 4-fluoro-phenyl]-4- methyl-7,8-dihydro- 6H-quinazolin-5-one | Rt = 2.124 m/z = 384.2 | C |
| 220 | 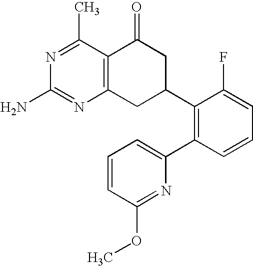 | 2-amino-7-[2-fluoro- 6-(6-methoxy- pyridin-2-yl)- phenyl]-4-methyl- 7,8-dihydro-6H- quinazolin-5-one | Rt = 2.56 m/z = 379.1 | C |
| 221 | 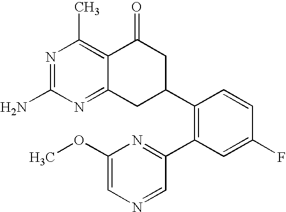 | 2-amino-7-[4-fluoro- 2-(6-methoxy- pyrazin-2-yl)- phenyl]-4-methyl- 7,8-dihydro-6H- quinazolin-5-one | Rt = 2.116 m/z = 380.0 | C |
| 222 | 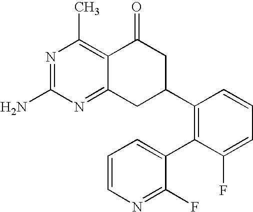 | 2-amino-7-[3-fluoro- 2-(2-fluoro-pyridin- 3-yl)-phenyl]-4- methyl-7,8-dihydro- 6H-quinazolin-5-one | Rt = 2.048 m/z = 367.0 | C |
| 223 | 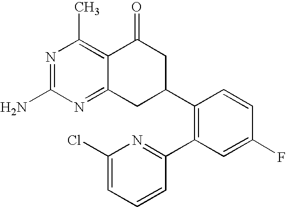 | 2-amino-7-[2-(6- chloro-pyridin-2-yl)- 4-fluoro-phenyl]-4- methyl-7,8-dihydro- 6H-quinazolin-5-one | Rt = 2.230 m/z = 382.9 | C |
| 224 | 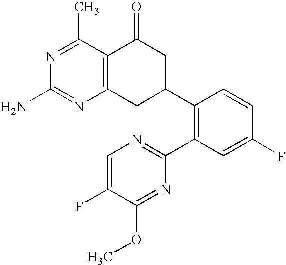 | 2-amino-7-[4-fluoro- 2-(5-fluoro-4- methoxy-pyrimidin- 2-yl)-phenyl]-4- methyl-7,8-dihydro- 6H-quinazolin-5-one | Rt = 2.25 m/z = 398.0 | C |
| 225 | 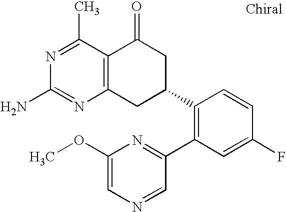 | (R)-2-amino-7-[4- fluoro-2-(6-methoxy- pyrazin-2-yl)- phenyl]-4-methyl- 7,8-dihydro-6H- quinazolin-5-one | Rt = 1.112 m/z = 380.0 | B |
| 226 | 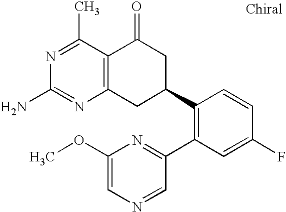 | (S)-2-amino-7-[4- fluoro-2-(6-methoxy- pyrazin-2-yl)- phenyl]-4-methyl- 7,8-dihydro-6H- quinazolin-5-one | Rt = 1.112 m/z = 380.0 | C |
| 227 | 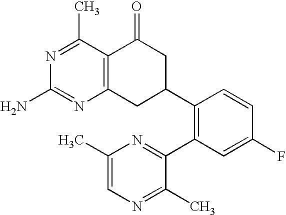 | 2-amino-7-[2-(3,6- dimethyl-pyrazin-2- yl)-4-fluoro-phenyl]- 4-methyl-7,8-dihydro- 6H-quinazolin-5-one | Rt = 1.864 m/z = 378.0 | B |
| 228 | 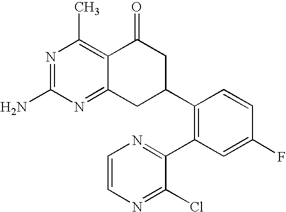 | 2-amino-7-[2-(3- chloro-pyrazin-2-yl)- 4-fluoro-phenyl]-4- methyl-7,8-dihydro- 6H-quinazolin-5-one | Rt = 2.031 m/z = 383.9 | B |
| 229 | 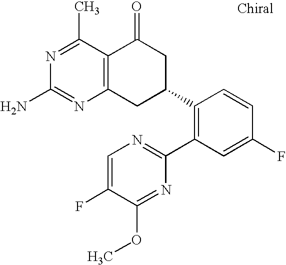 | (R)-2-amino-7-[4- fluoro-2-(5-fluoro-4- methoxy-pyrimidin- 2-yl)-phenyl]-4- methyl-7,8-dihydro- 6H-quinazolin-5-one | Rt = 2.25 m/z = 398.0 | B |
| 230 | 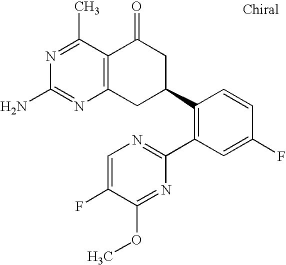 | (S)-2-amino-7-[4- fluoro-2-(5-fluoro-4- methoxy-pyrimidin- 2-yl)-phenyl]-4- methyl-7,8-dihydro- 6H-quinazolin-5-one | Rt = 2.25 m/z = 398.0 | C |
| 231 | 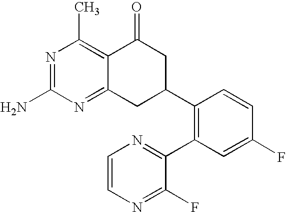 | 2-amino-7-[4-fluoro- 2-(3-fluoro-pyrazin- 2-yl)-phenyl]-4- methyl-7,8-dihydro- 6H-quinazolin-5-one | Rt = 2.013 m/z = 368.0 | C |
| 232 | 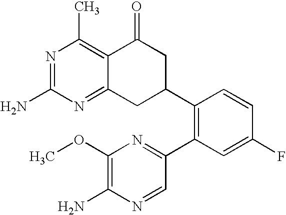 | 2-amino-7-[2-(5- amino-6-methoxy- pyrazin-2-yl)-4- fluoro-phenyl]-4- methyl-7,8-dihydro- 6H-quinazolin-5-one | Rt = 1.727 m/z = 395.0 | C |
| 233 | 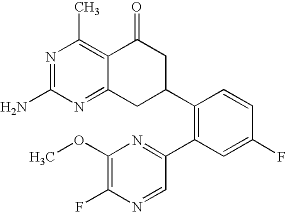 | 2-amino-7-[4-fluoro- 2-(5-fluoro-6- methoxy-pyrazin-2- yl)-phenyl]-4- methyl-7,8-dihydro- 6H-quinazolin-5-one | Rt = 2.354 m/z = 398.0 | C |
| 234 | 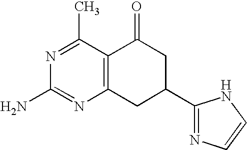 | 2-amino-7-(1H- imidazol-2-yl)-4- methyl-7,8-dihydro- 6H-quinazolin-5-one | Rt = 0.92 m/z = 244.0 | A |
| 235 | 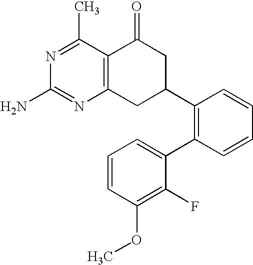 | 2-amino-7-(2′- fluoro-3′-methoxy- biphenyl-2-yl)-4- methyl-7,8-dihydro- 6H-quinazolin-5-one | Rt = 2.39 m/z = 378.4 | B |
| 236 | 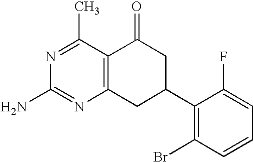 | 2-amino-7-(2-bromo- 6-fluoro-phenyl)-4- methyl-7,8-dihydro- 6H-quinazolin-5-one | Rt = 2.11 m/z = 352.1 | B |
| 237 | 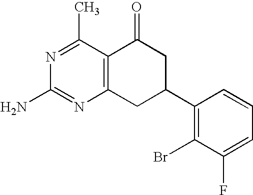 | 2-amino-7-(2-bromo- 3-fluoro-phenyl)-4- methyl-7,8-dihydro- 6H-quinazolin-5-one | Rt = 2.1 m/z = 350.1 | B |
| 238 | 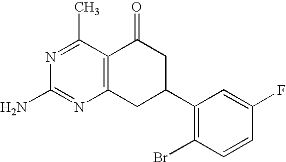 | 2-amino-7-(2-bromo- 5-fluoro-phenyl)-4- methyl-7,8-dihydro- 6H-quinazolin-5-one | Rt = 2.22 m/z = 352.1 | B |
| 239 | 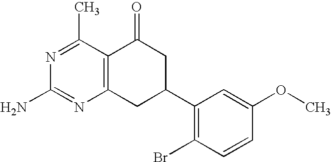 | 2-amino-7-(2-bromo- 5-methoxy-phenyl)- 4-methyl-7,8- dihydro-6H- quinazolin-5-one | Rt = 2.23 m/z = 362.1 | B |
| 240 | 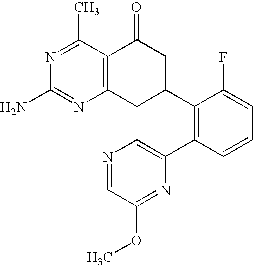 | 2-amino-7-[2-fluoro- 6-(6-methoxy- pyrazin-2-yl)- phenyl]-4-methyl- 7,8-dihydro-6H- quinazolin-5-one | Rt = 2.124 m/z = 401.0 (+Na) | B |
| 241 | 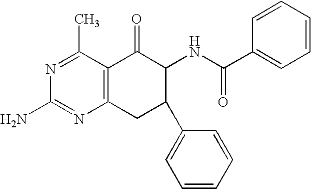 | N-(2-amino-4- methyl-5-oxo-7- phenyl-5,6,7,8- tetrahydro- quinazolin-6-yl)- benzamide | Rt = 2.22 m/z = 373.1 | A |
| 242 | 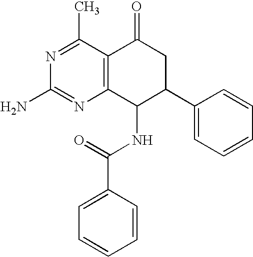 | N-(2-amino-4- methyl-5-oxo-7- phenyl-5,6,7,8- tetrahydro- quinazolin-8-yl)- benzamide | Rt = 2.27 m/z = 373.1 | A |
| 243 | 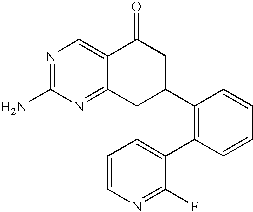 | 2-amino-7-[2-(2- fluoro-pyridin-3-yl)- phenyl]-7,8-dihydro- 6H-quinazolin-5-one | Rt = 2.24 m/z = 335.2 | C |
| 244 | 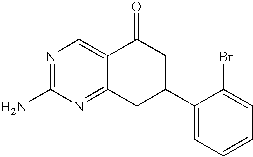 | 2-amino-7-(2-bromo- phenyl)-7,8-dihydro- 6H-quinazolin-5-one | Rt = 2.35 m/z = 320.0 | A |
| 245 | 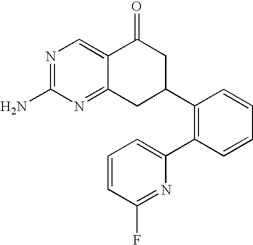 | 2-amino-7-[2-(6- fluoro-pyridin-2-yl)- phenyl]-7,8-dihydro- 6H-quinazolin-5-one | Rt = 2.32 m/z = 335.1 | C |
| 246 | 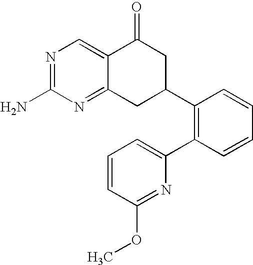 | 2-amino-7-[2-(6- methoxy-pyridin-2- yl)-phenyl]-7,8- dihydro-6H- quinazolin-5-one | Rt = 2.43 m/z = 347.1 | C |
| 247 | 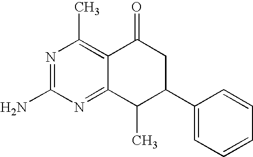 | 2-amino-4,8- dimethyl-7-phenyl- 7,8-dihydro-6H- quinazolin-5-one | Rt = 3.51 m/z = 268.2 | B |
| 248 | 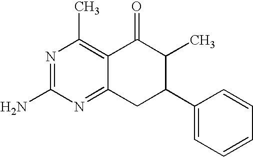 | 2-amino-4,6- dimethyl-7-phenyl- 7,8-dihydro-6H- quinazolin-5-one | Rt = 3.59 m/z = 268.2 | C |
| 249 | 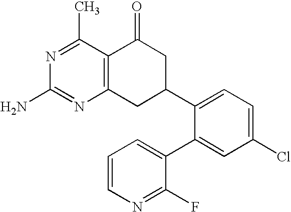 | 2-amino-7-[4-chloro- 2-(2-fluoro-pyridin- 3-yl)-phenyl]-4- methyl-7,8-dihydro- 6H-quinazolin-5-one | Rt = 2.50 m/z = 383.0 | C |
| 250 | 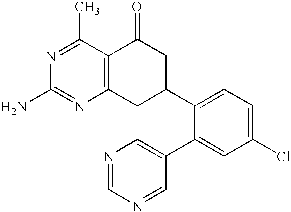 | 2-amino-7-(4-chloro- 2-pyrimidin-5-yl- phenyl)-4-methyl- 7,8-dihydro-6H- quinazolin-5-one | Rt = 2.17 m/z = 366.0 | C |
| 251 | 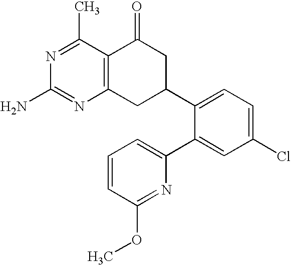 | 2-amino-7-[4-chloro- 2-(6-methoxy- pyridin-2-yl)- phenyl]-4-methyl- 7,8-dihydro-6H- quinazolin-5-one | Rt = 2.82 m/z = 395.0 | C |
| 252 | 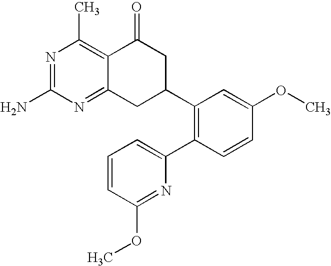 | 2-amino-7-[5- methoxy-2-(6- methoxy-pyridin-2- yl)-phenyl]-4- methyl-7,8-dihydro- 6H-quinazolin-5-one | Rt = 2.47 m/z = 391.1 | C |
| 253 | 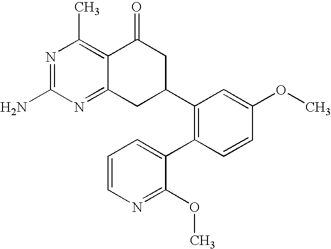 | 2-amino-7-[5- methoxy-2-(2- methoxy-pyridin-3- yl)-phenyl]-4- methyl-7,8-dihydro- 6H-quinazolin-5-one | Rt = 2.35 m/z = 391.1 | C |
| 254 | 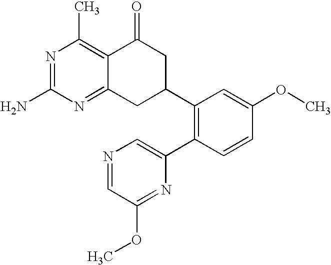 | 2-amino-7-[5- methoxy-2-(6- methoxy-pyrazin-2- yl)-phenyl]-4- methyl-7,8-dihydro- 6H-quinazolin-5-one | Rt = 2.32 m/z = 392.1 | C |
| 255 | 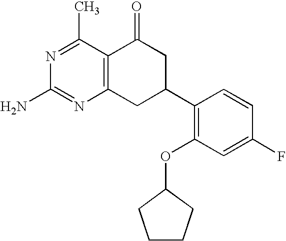 | 2-amino-7-(2- cyclopentyloxy-4- fluoro-phenyl)-4- methyl-7,8-dihydro- 6H-quinazolin-5-one | Rt = 4.86 m/z = 356.2 | C |
| 256 | 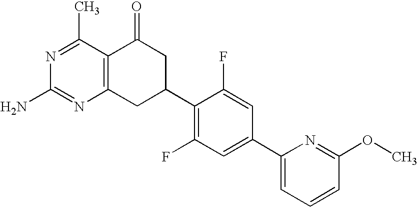 | 2-amino-7-[2,6- difluoro-4-(6- methoxy-pyridin-2- yl)-phenyl]-4- methyl-7,8-dihydro- 6H-quinazolin-5-one | Rt = 4.93 m/z = 397.0 | A |
| 257 | 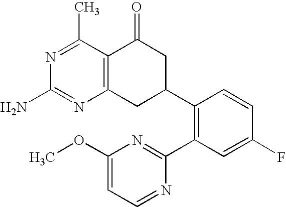 | 2-amino-7-[4-fluoro- 2-(4-methoxy- pyrimidin-2-yl)- phenyl]-4-methyl- 7,8-dihydro-6H- quinazolin-5-one | Rt = 2.54 m/z = 380.0 | C |
| 258 | 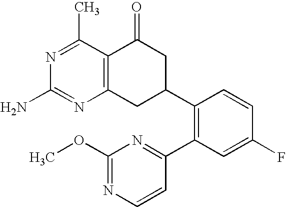 | 2-amino-7-[4-fluoro- 2-(2-methoxy- pyrimidin-4-yl)- phenyl]-4-methyl- 7,8-dihydro-6H- quinazolin-5-one | Rt = 3.11 m/z = 380.0 | A |
| 259 | 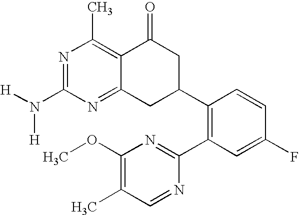 | 2-amino-7-[4-fluoro- 2-(4-methoxy-5- methyl-pyrimidin-2- yl)-phenyl]-4- methyl-7,8-dihydro- 6H-quinazolin-5-one | Rt = 3.15 m/z = 394.1 | C |
| 260 | 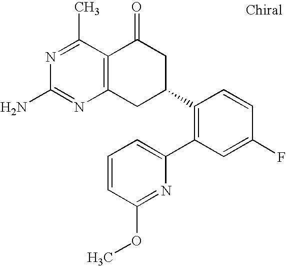 | (R)-2-amino-7-[4- fluoro-2-(6-methoxy- pyridin-2-yl)- phenyl]-4-methyl- 7,8-dihydro-6H- quinazolin-5-one | Rt = 3.20 m/z = 379.0 | B |
| 261 | 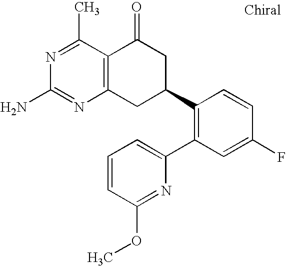 | (S)-2-amino-7-[4- fluoro-2-(6-methoxy- pyridin-2-yl)- phenyl]-4-methyl- 7,8-dihydro-6H- quinazolin-5-one | Rt = 3.20 m/z = 379.0 | C |
| 262 | 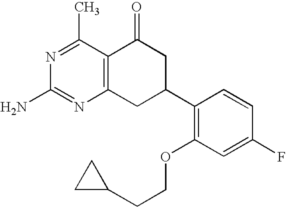 | 2-amino-7-[2-(2- cyclopropyl-ethoxy)- 4-fluoro-phenyl]-4- methyl-7,8-dihydro- 6H-quinazolin-5-one | Rt = 3.55 m/z = 356.0 | C |
| 263 | 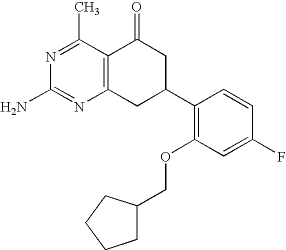 | 2-amino-7-(2- cyclopentylmethoxy- 4-fluoro-phenyl)-4- methyl-7,8-dihydro- 6H-quinazolin-5-one | Rt = 3.874 m/z = 370.1 | C |
| 264 | 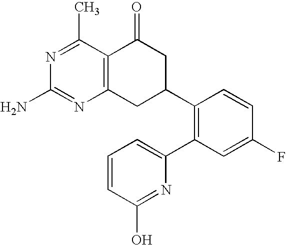 | 2-amino-7-[4-fluoro- 2-(6-hydroxy- pyridin-2-yl)- phenyl]-4-methyl- 7,8-dihydro-6H- quinazolin-5-one | Rt = 1.97 m/z = 365.0 | B |
| 265 | 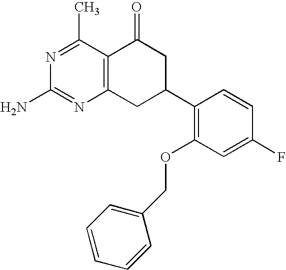 | 2-amino-7-(2- benzyloxy-4-fluoro- phenyl)-4-methyl- 7,8-dihydro-6H- quinazolin-5-one | Rt = 3.50 m/z = 378.1 | B |
| 266 | 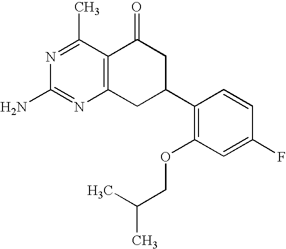 | 2-amino-7-(4-fluoro- 2-isobutoxy-phenyl)- 4-methyl-7,8- dihydro-6H- quinazolin-5-one | Rt = 3.55 m/z = 344.1 | C |
| 267 | 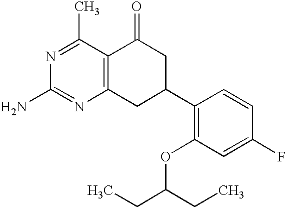 | 2-amino-7-[2-(1- ethyl-propoxy)-4- fluoro-phenyl]-4- methyl-7,8-dihydro- 6H-quinazolin-5-one | Rt = 3.71 m/z = 358.0 | C |
| 268 | 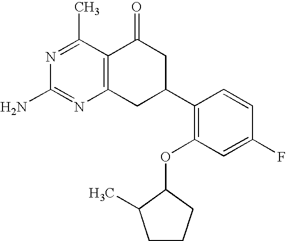 | 2-amino-7-[4-fluoro- 2-(2-methyl- cyclopentyloxy)- phenyl]-4-methyl- 7,8-dihydro-6H- quinazolin-5-one | Rt = 3.77 m/z = 370.1 | C |
| 269 | 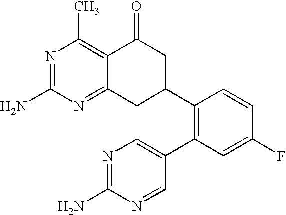 | 2-amino-7-[2-(2- amino-pyrimidin-5- yl)-4-fluoro-phenyl]- 4-methyl-7,8- dihydro-6H- quinazolin-5-one | Rt = 1.94 m/z = 365.0 | B |
| 270 | 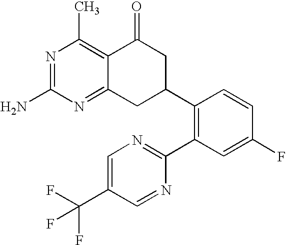 | 2-amino-7-[4-fluoro- 2-(5-trifluoromethyl- pyrimidin-2-yl)- phenyl]-4-methyl- 7,8-dihydro-6H- quinazolin-5-one | Rt = 3.17 m/z = 418.0 | C |
| 271 | 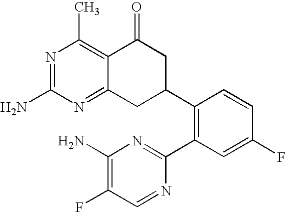 | 2-amino-7-[2-(4- amino-5-fluoro- pyrimidin-2-yl)-4- fluoro-phenyl]-4- methyl-7,8-dihydro- 6H-quinazolin-5-one | Rt = 1.78 m/z = 383.1 | |
| 272 | 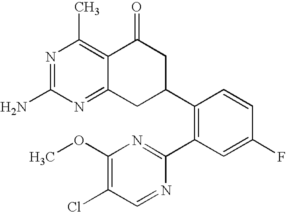 | 2-amino-7-[2-(5- chloro-4-methoxy- pyrimidin-2-yl)-4- fluoro-phenyl]-4- methyl-7,8-dihydro- 6H-quinazolin-5-one | Rt = 3.32 m/z = 414.1 | C |
| 273 | 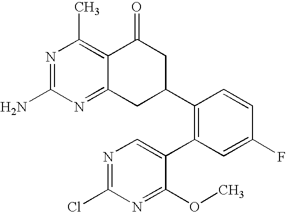 | 2-amino-7-[2-(2- chloro-4-methoxy- pyrimidin-5-yl)-4- fluoro-phenyl]-4- methyl-7,8-dihydro- 6H-quinazolin-5-one | Rt = 3.03 m/z = 414.0 | C |
| 274 | 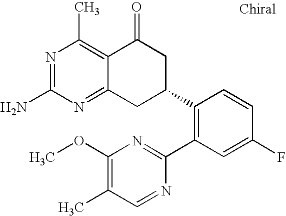 | (R)-2-amino-7-[4- fluoro-2-(4-methoxy- 5-methyl-pyrimidin- 2-yl)-phenyl]-4- methyl-7,8-dihydro- 6H-quinazolin-5-one | Rt = 3.15 m/z = 394.1 | C |
| 275 | 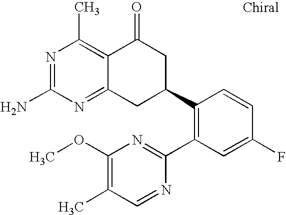 | (S)-2-amino-7-[4- fluoro-2-(4-methoxy- 5-methyl-pyrimidin- 2-yl)-phenyl]-4- methyl-7,8-dihydro- 6H-quinazolin-5-one | Rt = 3.15 m/z = 394.1 | C |
| 276 | 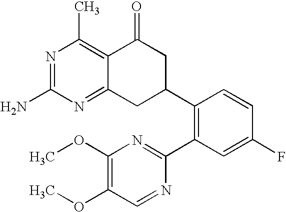 | 2-amino-7-[2-(4,5- dimethoxy- pyrimidin-2-yl)-4- fluoro-phenyl]-4- methyl-7,8-dihydro- 6H-quinazolin-5-one | Rt = 3.66 m/z = 410.1 | C |
| 277 | 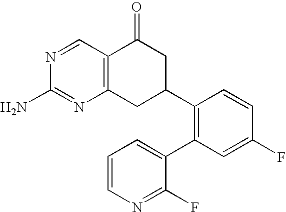 | 2-amino-7-[4-fluoro- 2-(2-fluoro-pyridin- 3-yl)-phenyl]-7,8- dihydro-6H- quinazolin-5-one | Rt = 2.33 m/z = 353.0 | C |
| 278 | 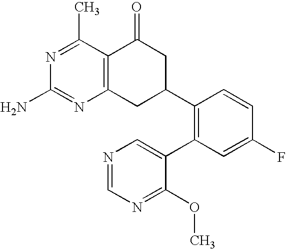 | 2-amino-7-[4-fluoro- 2-(4-methoxy- pyrimidin-5-yl)- phenyl]-4-methyl- 7,8-dihydro-6H- quinazolin-5-one | Rt = 2.09 m/z = 380.0 | C |
| 279 | 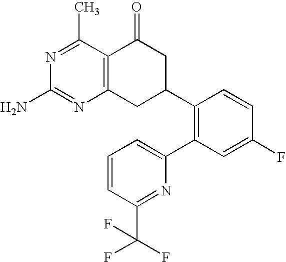 | 2-amino-7-[4-fluoro- 2-(6-trifluoromethyl- pyridin-2-yl)- phenyl]-4-methyl- 7,8-dihydro-6H- quinazolin-5-one | Rt = 2.64 m/z = 417.0 | C |
| 280 | 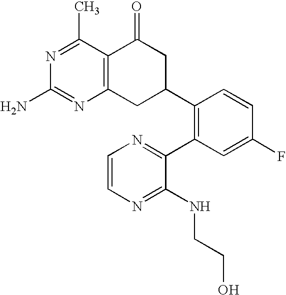 | 2-amino-7-{4-fluoro- 2-[3-(2-hydroxy- ethylamino)-pyrazin- 2-yl]-phenyl}-4- methyl-7,8-dihydro- 6H-quinazolin-5-one | Rt = 1.81 m/z = 409.0 | B |
| 281 | 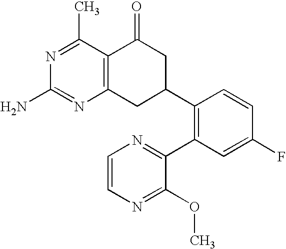 | 2-amino-7-[4-fluoro- 2-(3-methoxy- pyrazin-2-yl)- phenyl]-4-methyl- 7,8-dihydro-6H- quinazolin-5-one | Rt = 2.25 m/z = 380.0 | C |
| 282 | 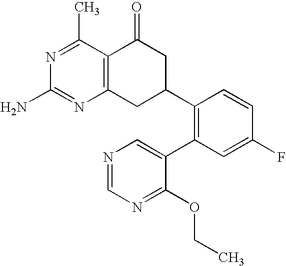 | 2-amino-7-[2-(4- ethoxy-pyrimidin-5- yl)-4-fluoro-phenyl]- 4-methyl-7,8- dihydro-6H- quinazolin-5-one | Rt = 2.21 m/z = 394.0 | C |
| 283 | 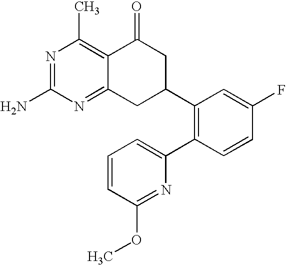 | 2-amino-7-[5-fluoro- 2-(6-methoxy- pyridin-2-yl)- phenyl]-4-methyl- 7,8-dihydro-6H- quinazolin-5-one | Rt = 2.59 m/z = 379.1 | C |
| 284 | 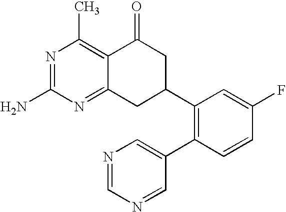 | 2-amino-7-(5-fluoro- 2-pyrimidin-5-yl- phenyl)-4-methyl- 7,8-dihydro-6H- quinazolin-5-one | Rt = 2.02 m/z = 350.1 | A |
| 285 | 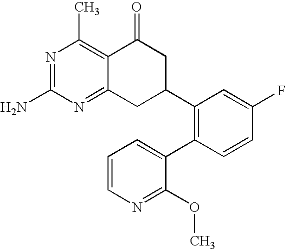 | 2-amino-7-[5-fluoro- 2-(2-methoxy- pyridin-3-yl)- phenyl]-4-methyl- 7,8-dihydro-6H- quinazolin-5-one | Rt = 2.44 m/z = 379.1 | C |
| 286 | 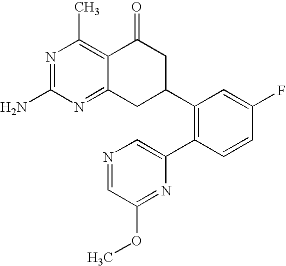 | 2-amino-7-[5-fluoro- 2-(6-methoxy- pyrazin-2-yl)- phenyl]-4-methyl- 7,8-dihydro-6H- quinazolin-5-one | Rt = 2.38 m/z = 380.1 | B |
| 287 | 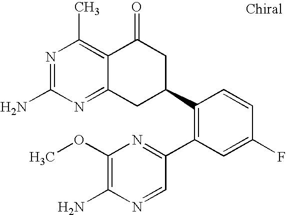 | (S)-2-amino-7-[2-(5- amino-6-methoxy- pyrazin-2-yl)-4- fluoro-phenyl]-4- methyl-7,8-dihydro- 6H-quinazolin-5-one | Rt = 1.727 m/z = 395.0 | C |
| 288 | 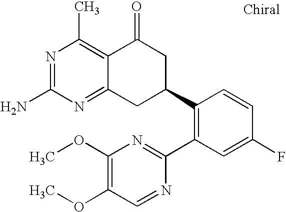 | (S)-2-amino-7-[2- (4,5-dimethoxy- pyrimidin-2-yl)-4- fluoro-phenyl]-4- methyl-7,8-dihydro- 6H-quinazolin-5-one | Rt = 2.31 m/z = 410.1 | C |
| 289 | 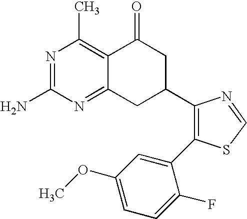 | 2-amino-7-[5-(2- fluoro-5-methoxy- phenyl)-thiazol-4- yl]-4-methyl-7,8- dihydro-6H- quinazolin-5-one | Rt = 2.79 m/z = 385.1 | B |
| 290 | 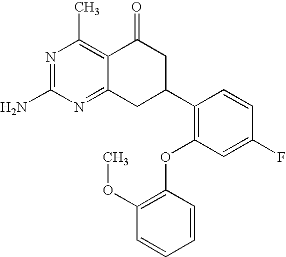 | 2-amino-7-[4-fluoro- 2-(2-methoxy- phenoxy)-phenyl]-4- methyl-7,8-dihydro- 6H-quinazolin-5-one | Rt = 3.41 m/z = 394.1 | B |
| 291 | 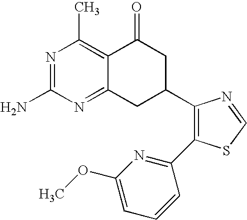 | 2-amino-7-[5-(6- methoxy-pyridin-2- yl)-thiazol-4-yl]-4- methyl-7,8-dihydro- 6H-quinazolin-5-one | Rt = 2.71 m/z = 368.2 | C |
| 292 | 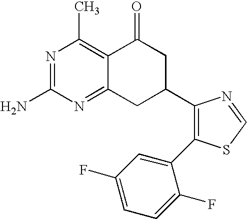 | 2-amino-7-[5-(2,5- difluoro-phenyl)- thiazol-4-yl]-4- methyl-7,8-dihydro- 6H-quinazolin-5-one | Rt = 2.84 m/z = 373.1 | C |
| 293 | 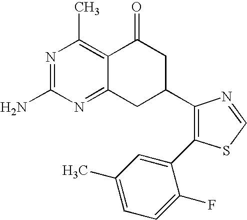 | 2-amino-7-[5-(2- fluoro-5-methyl- phenyl)-thiazol-4- yl]-4-methyl-7,8- dihydro-6H- quinazolin-5-one | Rt = 2.94 m/z = 369.0 | C |
| 294 | 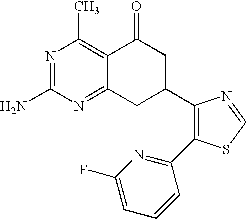 | 2-amino-7-[5-(6- fluoro-pyridin-2-yl)- thiazol-4-yl]-4- methyl-7,8-dihydro- 6H-quinazolin-5-one | Rt = 2.45 m/z = 356.1 | C |
| 295 | 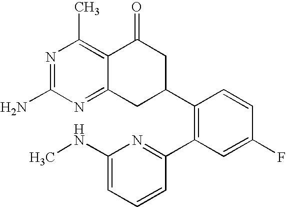 | 2-amino-7-[4-fluoro- 2-(6-methylamino- pyridin-2-yl)- phenyl]-4-methyl- 7,8-dihydro-6H- quinazolin-5-one | Rt = 1.83 m/z = 378.2 | B |
| 296 | 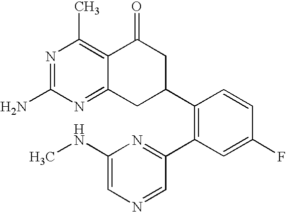 | 2-amino-7-[4-fluoro- 2-(6-methylamino- pyrazin-2-yl)- phenyl]-4-methyl- 7,8-dihydro-6H- quinazolin-5-one | Rt = 2.35 m/z = 379.1 | C |
| 297 | 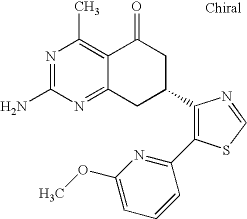 | (R)-2-amino-7-[5-(6- methoxy-pyridin-2- yl)-thiazol-4-yl]-4- methyl-7,8-dihydro- 6H-quinazolin-5-one | Rt = 2.72 m/z = 368.1 | B |
| 298 | 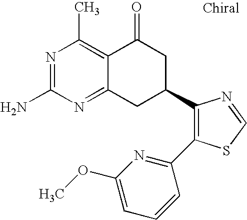 | (S)-2-amino-7-[5-(6- methoxy-pyridin-2- yl)-thiazol-4-yl]-4- methyl-7,8-dihydro- 6H-quinazolin-5-one | Rt = 2.72 m/z = 368.1 | C |
| 299 | 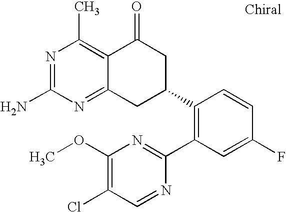 | (R)-2-amino-7-[2-(5- chloro-4-methoxy- pyrimidin-2-yl)-4- fluoro-phenyl]-4- methyl-7,8-dihydro- 6H-quinazolin-5-one | Rt = 3.31 m/z = 414.1 | C |
| 300 | 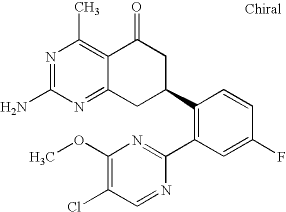 | (S)-2-amino-7-[2-(5- chloro-4-methoxy- pyrimidin-2-yl)-4- fluoro-phenyl]-4- methyl-7,8-dihydro- 6H-quinazolin-5-one | Rt = 3.31 m/z = 414.1 | C |
| 301 | 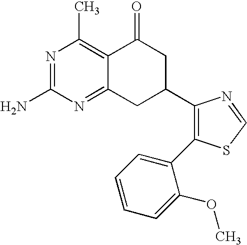 | 2-amino-7-[5-(2- methoxy-phenyl)- thiazol-4-yl]-4- methyl-7,8-dihydro- 6H-quinazolin-5-one | Rt = 2.66 m/z = 367.1 | C |
| 302 | 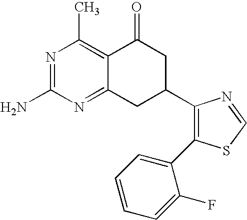 | 2-amino-7-[5-(2- fluoro-phenyl)- thiazol-4-yl]-4- methyl-7,8-dihydro- 6H-quinazolin-5-one | Rt = 2.65 m/z = 355.1 | C |
| 303 | 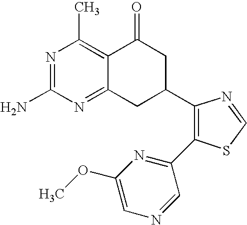 | 2-amino-7-[5-(6- methoxy-pyrazin-2- yl)-thiazol-4-yl]-4- methyl-7,8-dihydro- 6H-quinazolin-5-one | Rt = 2.20 m/z = 369.1 | C |
| 304 | 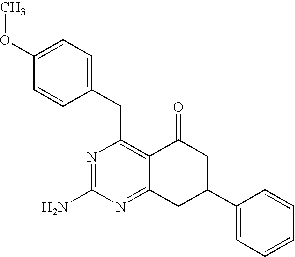 | 2-amino-4-(4- methoxy-benzyl)-7- phenyl-7,8-dihydro- 6H-quinazolin-5-one | Rt = 2.6 m/z = 360 | B |
| 305 | 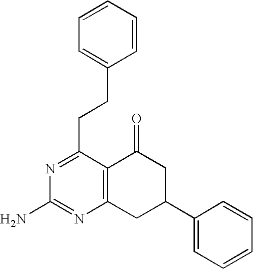 | 2-amino-4- phenethyl-7-phenyl- 7,8-dihydro-6H- quinazolin-5-one | Rt = 2.72 m/z = 344.3 | A |
| 306 | 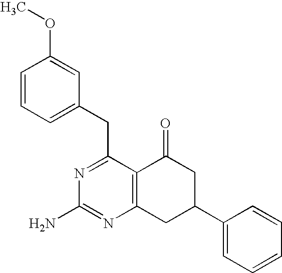 | 2-amino-4-(3- methoxy-benzyl)-7- phenyl-7,8-dihydro- 6H-quinazolin-5-one | Rt = 2.63 m/z = 360.3 | B |
| 307 | 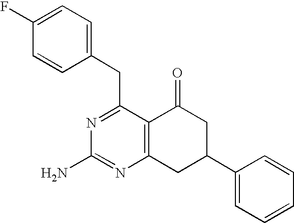 | 2-amino-4-(4-fluoro- benzyl)-7-phenyl- 7,8-dihydro-6H- quinazolin-5-one | Rt = 2.71 m/z = 348.2 | B |
| 308 | 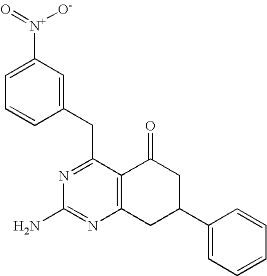 | 2-amino-4-(3-nitro- benzyl)-7-phenyl- 7,8-dihydro-6H- quinazolin-5-one | Rt = 2.73 m/z = 375.2 | A |
| 309 | 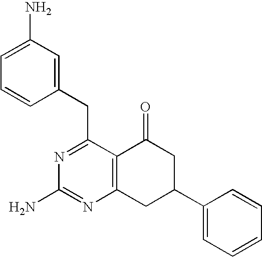 | 2-amino-4-(3-amino- benzyl)-7-phenyl- 7,8-dihydro-6H- quinazolin-5-one | Rt = 1.85 m/z = 345.2 | A |
| 310 | 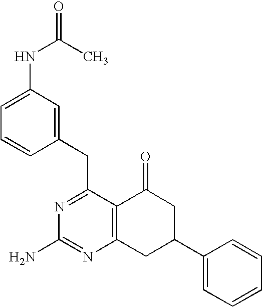 | N-[3-(2-amino-5- oxo-7-phenyl- 5,6,7,8-tetrahydro- quinazolin-4- ylmethyl)-phenyl]- acetamide | Rt = 2.21 m/z = 387.3 | A |
| 311 | 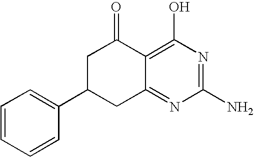 | 2-amino-4-hydroxy- 7-phenyl-7,8- dihydro-6H- quinazolin-5-one | Rt = 1.57 m/z = 256.1 | A |
| 312 | 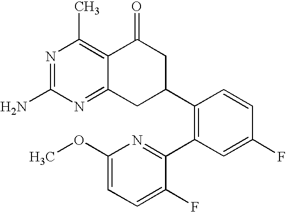 | 2-amino-7-[4-fluoro- 2-(3-fluoro-6- methoxy-pyridin-2- yl)-phenyl]-4- methyl-7,8-dihydro- 6H-quinazolin-5-one | | |
| 313 | 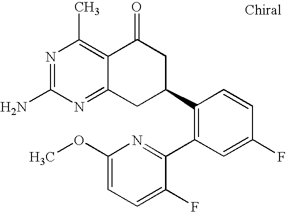 | (S)-2-amino-7-[4- fluoro-2-(3-fluoro-6- methoxy-pyridin-2- yl)-phenyl]-4- methyl-7,8-dihydro- 6H-quinazolin-5-one | | C |
| 314 | 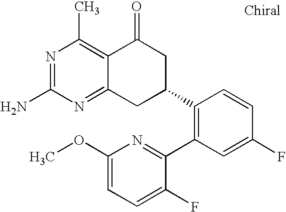 | (R)-2-amino-7-[4- fluoro-2-(3-fluoro-6- methoxy-pyridin-2- yl)-phenyl]-4- methyl-7,8-dihydro- 6H-quinazolin-5-one | | B |
| 315 | 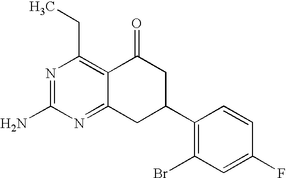 | 2-amino-7-(2-bromo- 4-fluoro-phenyl)-4- ethyl-7,8-dihydro- 6H-quinazolin-5-one | Rt = 2.64 m/z = 363.9 | A |
| 316 | 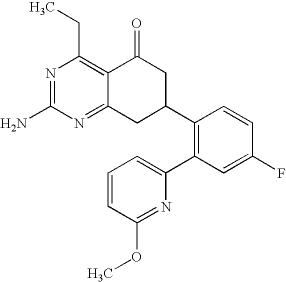 | 2-amino-4-ethyl-7- [4-fluoro-2-(6- methoxy-pyridin-2- yl)-phenyl]-7,8- dihydro-6H- quinazolin-5-one | Rt = 2.75 m/z = 393.1 | C |
| 317 | 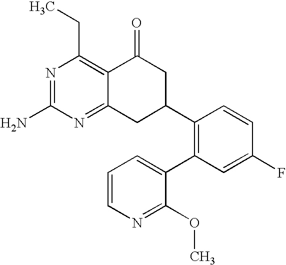 | 2-amino-4-ethyl-7- [4-fluoro-2-(2- methoxy-pyridin-3- yl)-phenyl]-7,8- dihydro-6H- quinazolin-5-one | Rt = 2.61 m/z = 393.1 | B |
| 318 | 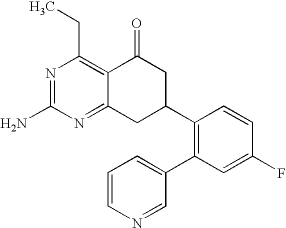 | 2-amino-4-ethyl-7- (4-fluoro-2-pyridin- 3-yl-phenyl)-7,8- dihydro-6H- quinazolin-5-one | Rt = 1.67 m/z = 363.0 | C |
| 319 | 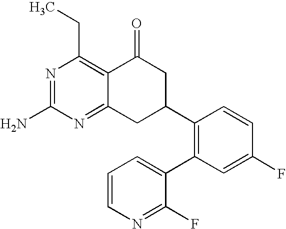 | 2-amino-4-ethyl-7- [4-fluoro-2-(2- fluoro-pyridin-3-yl)- phenyl]-7,8-dihydro- 6H-quinazolin-5-one | Rt = 2.26 m/z = 381.0 | B |
| 320 | 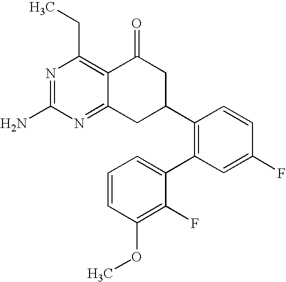 | 2-amino-7-(5,2′- difluoro-3′-methoxy- biphenyl-2-yl)-4- ethyl-7,8-dihydro- 6H-quinazolin-5-one | Rt = 2.59 m/z = 410.0 | A |
| 321 | 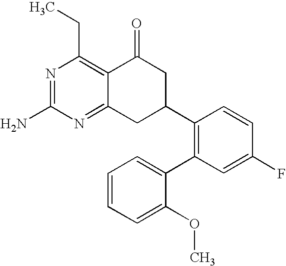 | 2-amino-4-ethyl-7- (5-fluoro-2′- methoxy-biphenyl-2- yl)-7,8-dihydro-6H- quinazolin-5-one | Rt = 2.60 m/z = 392.0 | B |
| 322 | 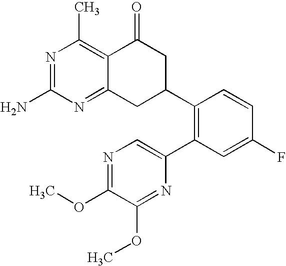 | 2-amino-7-[2-(5,6- dimethoxy-pyrazin- 2-yl)-4-fluoro- phenyl]-4-methyl- 7,8-dihydro-6H- quinazolin-5-one | Rt = 2.35 m/z = 410.0 | C |
| 323 | 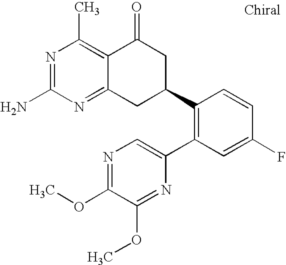 | (S)-2-amino-7-[2- (5,6-dimethoxy- pyrazin-2-yl)-4- fluoro-phenyl]-4- methyl-7,8-dihydro- 6H-quinazolin-5-one | Rt = 2.59 m/z = 410.1 | C |
| 324 | 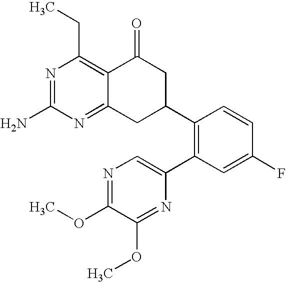 | 2-amino-7-[2-(5,6- dimethoxy-pyrazin- 2-yl)-4-fluoro- phenyl]-4-ethyl-7,8- dihydro-6H- quinazolin-5-one | Rt = 2.74 m/z = 424.2 | C |
| 325 | 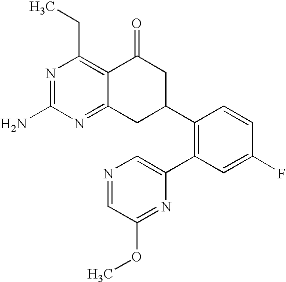 | 2-amino-4-ethyl-7- [4-fluoro-2-(6- methoxy-pyrazin-2- yl)-phenyl]-7,8- dihydro-6H- quinazolin-5-one | Rt = 2.55 m/z = 394.1 | C |
| 326 | 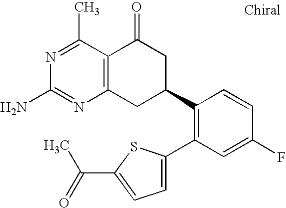 | (S)-7-[2-(5-Acetyl- thiophen-2-yl)-4- fluoro-phenyl]-2- amino-4-methyl-7,8- dihydro-6H- quinazolin-5-one | Rt = 2.50 m/z = 396.1 | B |
| 327 | 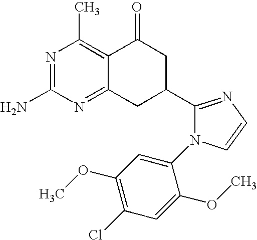 | 2-amino-7-[1-(4- chloro-2,5- dimethoxy-phenyl)- 1H-imidazol-2-yl]-4- methyl-7,8-dihydro- 6H-quinazolin-5-one | Rt = 2.00 m/z = 414 | C |
| 328 | 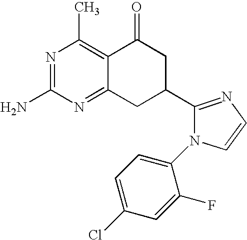 | 2-amino-7-[1-(4- chloro-2-fluoro- phenyl)-1H- imidazol-2-yl]-4- methyl-7,8-dihydro- 6H-quinazolin-5-one | Rt = 1.87 m/z = 372 | B |
| 329 | 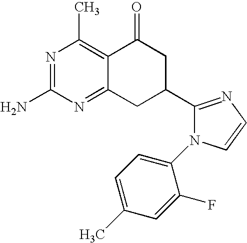 | 2-amino-7-[1-(2- fluoro-4-methyl- phenyl)-1H- imidazol-2-yl]-4- methyl-7,8-dihydro- 6H-quinazolin-5-one | Rt = 1.84 m/z = 352 | B |
| 330 | 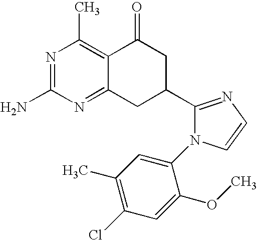 | 2-amino-7-[1-(4- chloro-2-methoxy-5- methyl-phenyl)-1H- imidazol-2-yl]-4- methyl-7,8-dihydro- 6H-quinazolin-5-one | Rt = 2.08 m/z = 398 | B |
| 331 | 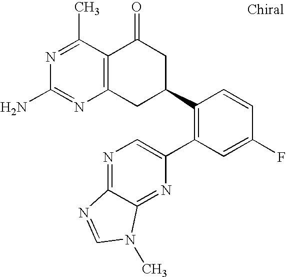 | (S)-2-amino-7-[4- fluoro-2-(3-methyl- 3H-imidazo[4,5- b]pyrazin-5-yl)- phenyl]-4-methyl- 7,8-dihydro-6H- quinazolin-5-one | Rt = 1.824 m/z = 404.2 | A |
| 332 | 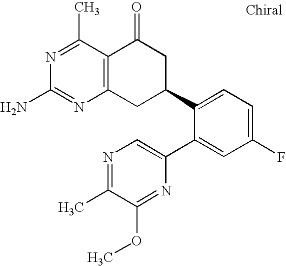 | (S)-2-amino-7-[4- fluoro-2-(6-methoxy- 5-methyl-pyrazin-2- yl)-phenyl]-4- methyl-7,8-dihydro- 6H-quinazolin-5-one | Rt = 2.48 m/z = 394.1 | C |
| 333 | 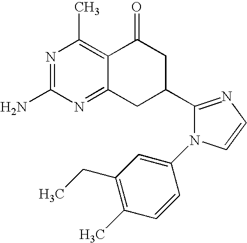 | 2-amino-7-[1-(3- ethyl-4-methyl- phenyl)-1H- imidazol-2-yl]-4- methyl-7,8-dihydro- 6H-quinazolin-5-one | Rt = 2.11 m/z = 362 | B |
| 334 | 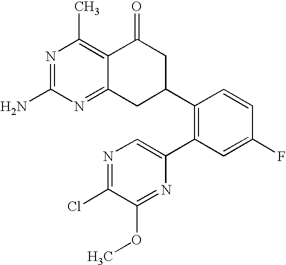 | 2-amino-7-[2-(5- chloro-6-methoxy- pyrazin-2-yl)-4- fluoro-phenyl]-4- methyl-7,8-dihydro- 6H-quinazolin-5-one | Rt = 2.59 m/z = 414.0 | C |
| 335 | 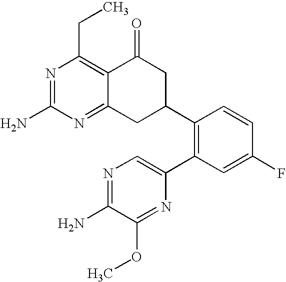 | 2-amino-7-[2-(5- amino-6-methoxy- pyrazin-2-yl)-4- fluoro-phenyl]-4- ethyl-7,8-dihydro- 6H-quinazolin-5-one | Rt = 2.11 m/z = 409.1 | C |
| 336 | 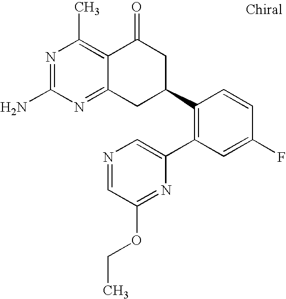 | (S)-2-amino-7-[2-(6- ethoxy-pyrazin-2- yl)-4-fluoro-phenyl]- 4-methyl-7,8- dihydro-6H- quinazolin-5-one | Rt = 2.53 m/z = 394.1 | C |
| 337 | 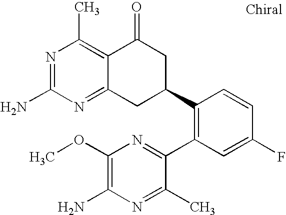 | (S)-2-amino-7-[2-(5- amino-6-methoxy-3- methyl-pyrazin-2- yl)-4-fluoro-phenyl]- 4-methyl-7,8- dihydro-6H- quinazolin-5-one | Rt = 1.74 m/z = 409.1 | C |

Using the procedure described in Example 22, certain compounds in Table 1 were shown to have HSP90 inhibitory activity at an IC₅₀ of less than 25 μM. Some of the compounds have an IC₅₀ of less than about 10 μM, others less than about 1 μM, and certain others of the compounds have an IC₅₀ of less than about 0.1 μM.

### Example 22

HSP90 Inhibitor Binding Potency: TRF Binding Assay

In this example, the binding potency of HSP90 inhibitors as measured by a TRF binding assay is described.

TRF competition binding assays were performed to determine the binding potency (IC₅₀ values) of HSP90 inhibitors. Purified His-tagged N-terminal ATP binding domain (amino acid residues 9-236) of HSP90α (HSP90α GeneID: 3320; mRNA Sequence NM_{—}005348) was incubated for two hours at room temperature in binding buffer (50 mM HEPES, 6 mM MgCl₂, 20 mM KCl and 0.1% BSA) with biotinylated radicicol and progressively higher concentrations of the competing compounds. A fraction of the mixture was transferred to capture plates (coated with streptavidin) and incubated for one hour at room temperature. After washing with DELFIA wash buffer, europium-labeled anti-his antibody was added and incubated for two hours at room temperature, followed by washing with DELFIA buffer. DELFIA enhancement solution was then added. After gentle shaking for 10 minutes, the plates were read in VICTOR for europium counts.

Note: IC₅₀ values can also be determined using published methods in the following references:
1. Carreras, C. W., A. Schirmer, et al. (2003). “Filter binding assay for the geldanamycin-heat shock protein 90 interaction.” Anal Biochem 317(1): 40-6;
2. Kim, J., S. Felts, et al. (2004). “Development of a fluorescence polarization assay for the molecular chaperone Hsp90.” J Biomol Screen 9(5): 375-81; and
3. Zhou, V., S. Han, et al. (2004). “A time-resolved fluorescence resonance energy transfer-based HTS assay and a surface plasmon resonance-based binding assay for heat shock protein 90 inhibitors.” Anal Biochem 331(2): 349-57.

While the preferred embodiment of the invention has been illustrated and described, it will be appreciated that various changes can be made therein without departing from the spirit and scope of the invention.

What is claimed is:

1. A compound having formula (I): 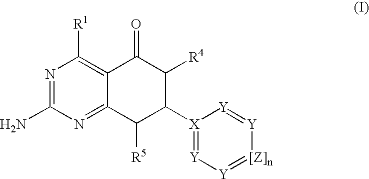
or a stereoisomer, tautomer, or pharmaceutically acceptable salt thereof, wherein
n is 0 or 1;
wherein when n is 1, X is C, Y is at each position independently selected from CQ¹ and N, and Z is selected from CR² and N, and
wherein when n is 0, X is C or N, Y is at each position independently selected from CQ¹, N, NQ², O, and S;
wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;
wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;
wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkyl amino,
(9) substituted or unsubstituted aryl,
(10) substituted or unsubstituted heteroaryl, and
(11) substituted or unsubstituted heterocyclyl;
wherein R² is selected from the group consisting of
(1) hydrogen,
(2) halogen,
(3) substituted or unsubstituted C₁-C₆ alkyl, and
(4) —OR³, —SR³, or —N(R³)₂;
wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;
wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino; and
with the proviso that when R¹ is methyl, and R⁴ and R⁵ are hydrogen, then X, Y, Z, and n together do not form an unsubstituted phenyl or furan-2-yl ring, and
with the proviso that when R¹, R⁴, and R⁵ are hydrogen, then X, Y, Z, and n together do not form a furan-2-yl, thien-2-yl, or phenyl ring wherein said ring is unsubstituted or substituted with one, two, or three substituents independently selected from the group consisting of C₁-C₆ alkyl, C₁-C₆ alkoxy, amino, alkylamino, dialkylamino, hydroxyl, and halo.

2. A compound of claim 1, wherein R¹ is hydrogen or substituted or unsubstituted C₁-C₆ alkyl.

3. A compound of claim 2, wherein R¹ is methyl.

4. A compound of claim 1, wherein R² is hydrogen or fluoro.

5. A compound of claim 1, wherein R⁴ is hydrogen.

6. A compound of claim 1, wherein R⁵ is hydrogen.

7. A compound according to claim 1, wherein at least one of Q¹, Q², R², or R³ is selected from the group consisting of substituted or unsubstituted aryl, substituted or unsubstituted heterocyclyl, substituted or unsubstituted heteroaryl, substituted or unsubstitued C₃-C₇ cycloalkyl, and substituted or unsubstitued C₅-C₇ cycloalkenyl.

8. A compound of claim 7, wherein said aryl, heterocyclyl, heteroaryl, C₃-C₇ cycloalkyl, and C₅-C₇ cycloalkenyl is selected from the group consisting of phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

9. A compound of claim 8, wherein one of Q¹ or Q² is selected from the group consisting of (2-hydroxy-ethylamino)-pyrazin-2-yl, 1-methyl-1H-pyrazol-4-yl, 2-(5-methyl-pyridin-2-yl)-phenyl, 2,3-difluorophenyl, 2,4-difluorophenyl, 2,4-dimethoxyphenyl, 2,5-difluorophenyl, 2,6-difluorophenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-3-yl, 2-chloro-pyridin-4-yl, 2-difluoro-3-methoxyphenyl, 2-ethyl-phenyl, 2-fluoro-3-methoxy-phenyl, 2-fluoro-3-methylphenyl, 2-fluoro-4-methylphenyl, 2-fluoro-4-methyl-phenyl, 2-fluoro-5-methoxy-phenyl, 2-fluoro-5-methoxy-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-methoxy-5-trifluoromethyl-phenyl, 2-methoxyphenyl, 2-methoxy-pyridin-3-yl, 2-methoxy-pyrimidin-4-yl, 2-methylphenyl, 2-methyl-pyridin-3-yl, 2-oxo-1,2-dihydro-pyridin-3-yl, 2-phenoxyphenyl, 2-trifluoromethoxyphenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethoxy-phenyl, 3-trifluoromethylphenyl, 4,5-dimethoxy-pyrimidin-2-yl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-ethoxy-pyrimidin-5-yl, 4-ethyl-1H-pyrazol-3-yl, 4-fluorophenyl, 4-methoxy-5-methyl-pyrimidin-2-yl, 4-methoxy-5-methyl-pyrimidin-2-yl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-pyridin-2-yl, 4-methyl-pyridin-3-yl, 5,6-dimethoxy-pyrazin-2-yl, 5-acetyl-thiophen-2-yl, 5-amino-6-methoxy-3-methyl-pyrazin-2-yl, 5-amino-6-methoxy-pyrazin-2-yl, 5-chloro-4-methoxy-pyrimidin-2-yl, 5-chloro-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-trifluoromethyl-pyrimidin-2-yl, 6-acetyl-pyridin-2-yl, 6-chloro-pyrazin-2-yl, 6-ethoxy-pyrazin-2-yl, 6-ethyl-pyridin-2-yl, 6-fluoro-pyridin-2-yl, 6-fluoro-pyridin-3-yl, 6-hydroxy-pyridin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, and 6-trifluoromethyl-pyridin-2-yl.

10. A compound of claim 1, wherein R³ is selected from the group consisting of methyl, ethyl, isopropyl, cyclopentyl, and cyclohexyl.

11. A compound of claim 1, wherein R³ is selected from the group consisting of substituted and unsubstituted phenyl, substituted and unsubstituted thiazolyl, substituted and unsubstituted pyridyl, substituted and unsubstituted pyrazinyl, and substituted and unsubstituted pyrimidinyl.

12. A compound of claim 1, wherein R³ is selected from the group consisting of 2-aminoethyl, 2-piperidinylethyl, 2-piperazinylethyl, 2-morpholinylethyl, and 2-(N-methylpiperazinyl)ethyl.

13. A compound of claim 1 having formula (Ia) 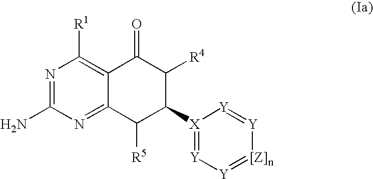
wherein R¹, R⁴, R⁵, X, Y, Z, and n are previously defined.

14. A compound of claim 1 having formula II 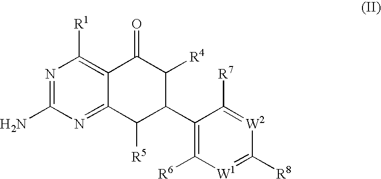
wherein W¹ and W² are independently N or CQ¹;
wherein R⁶ is selected from the group consisting of
(1) substituted or unsubstituted C₃-C₇ cycloalkyl,
(2) substituted or unsubstituted C₅-C₇ cycloalkenyl,
(3) substituted or unsubstituted aryl,
(4) substituted or unsubstituted heteroaryl, and
(5) substituted or unsubstituted heterocyclyl;
wherein R⁷ and R⁸ are independently
(1) hydrogen,
(2) halogen,
(3) substituted or unsubstituted C₁-C₆ alkyl,
(4) —OR³, —SR³, or —N(R³)₂, and
wherein Q¹, R¹, R³, R⁴, and R⁵ are previously defined.

15. A compound of claim 14, wherein R¹ is hydrogen or substituted or unsubstituted C₁-C₆ alkyl.

16. A compound of claim 15, wherein R¹ is methyl.

17. A compound of claim 14, wherein R⁴ is hydrogen.

18. A compound of claim 14, wherein R⁵ is hydrogen.

19. A compound of claim 14, wherein W¹ is N.

20. A compound of claim 14, wherein W² is N.

21. A compound of claim 14, wherein W¹ and W² are CQ¹.

22. A compound of claim 21, wherein each Q¹ is hydrogen.

23. A compound of claim 14, wherein R⁶ is selected from the group consisting of substituted aryl, substituted heterocyclyl, substituted heteroaryl, substituted C₃-C₇ cycloalkyl, and substituted C₅-C₇ cycloalkenyl, wherein said aryl, heterocyclyl, heteroaryl, C₃-C₇ cycloalkyl, and C₅-C₇ cycloalkenyl is selected from the group consisting of phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

24. A compound of claim 14, wherein R⁶ is selected from the group consisting of (2-hydroxy-ethylamino)-pyrazin-2-yl, 1-methyl-1H-pyrazol-4-yl, 2-(5-methyl-pyridin-2-yl)-phenyl, 2,3-difluorophenyl, 2,4-difluorophenyl, 2,4-dimethoxyphenyl, 2,5-difluorophenyl, 2,6-difluorophenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-3-yl, 2-chloro-pyridin-4-yl, 2-difluoro-3-methoxyphenyl, 2-ethyl-phenyl, 2-fluoro-3-methoxy-phenyl, 2-fluoro-3-methylphenyl, 2-fluoro-4-methylphenyl, 2-fluoro-4-methyl-phenyl, 2-fluoro-5-methoxy-phenyl, 2-fluoro-5-methoxy-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-methoxy-5-trifluoromethyl-phenyl, 2-methoxyphenyl, 2-methoxy-pyridin-3-yl, 2-methoxy-pyrimidin-4-yl, 2-methylphenyl, 2-methyl-pyridin-3-yl, 2-oxo-1,2-dihydro-pyridin-3-yl, 2-phenoxyphenyl, 2-trifluoromethoxyphenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethoxy-phenyl, 3-trifluoromethylphenyl, 4,5-dimethoxy-pyrimidin-2-yl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-ethoxy-pyrimidin-5-yl, 4-ethyl-1H-pyrazol-3-yl, 4-fluorophenyl, 4-methoxy-5-methyl-pyrimidin-2-yl, 4-methoxy-5-methyl-pyrimidin-2-yl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-pyridin-2-yl, 4-methyl-pyridin-3-yl, 5,6-dimethoxy-pyrazin-2-yl, 5-acetyl-thiophen-2-yl, 5-amino-6-methoxy-3-methyl-pyrazin-2-yl, 5-amino-6-methoxy-pyrazin-2-yl, 5-chloro-4-methoxy-pyrimidin-2-yl, 5-chloro-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-trifluoromethyl-pyrimidin-2-yl, 6-acetyl-pyridin-2-yl, 6-chloro-pyrazin-2-yl, 6-ethoxy-pyrazin-2-yl, 6-ethyl-pyridin-2-yl, 6-fluoro-pyridin-2-yl, 6-fluoro-pyridin-3-yl, 6-hydroxy-pyridin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, and 6-trifluoromethyl-pyridin-2-yl.

25. A compound of claim 14, wherein R⁷ is hydrogen.

26. A compound of claim 14, wherein R⁸ is hydrogen or fluoro.

27. A compound of claim 14 having formula (IIa) 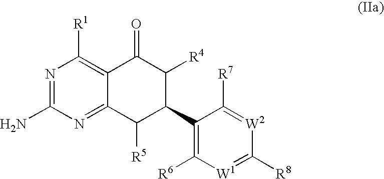
wherein R¹, R⁴, R⁵, R⁶, R⁷, R⁸, W¹, and W² are previously defined.

28. A compound of claim 1 having formula III: 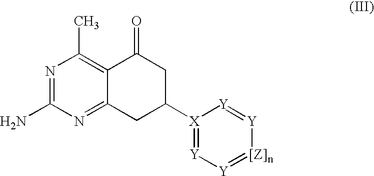
or a stereoisomer, tautomer, or pharmaceutically acceptable salt thereof, wherein
n is 0 or 1,
wherein when n is 1, X is C, Y is at each position independently selected from CQ¹ and N, and Z is selected from CR² and N, and
wherein when n is 0, X is C or N, Y is at each position independently selected from CQ¹, N, NQ², O, and S;
wherein Q¹ is selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted and unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;
wherein Q² is selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;
wherein R² is selected from the group consisting of
(1) hydrogen,
(2) halogen,
(3) substituted or unsubstituted C₁-C₃ alkyl,
(4) halo-substituted or unsubstituted —OCH₃, —SCH₃, or —NHCH₃, and
wherein R³ is at each position independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted and unsubstituted amino;
with the proviso that when n is 1, X is C, Y is CQ¹, and Z is CR², Q¹, and R² are not both hydrogen,
with the proviso that when n is 0, X is C, and Y adjacent to X is not O,
and with a further proviso that the total molecular weight does not exceed 750 Daltons.

29. A compound of claim 1 having formula (IV) 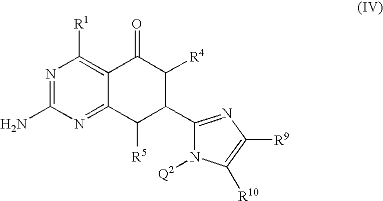
wherein R⁹ and R¹⁰ are independently Q¹, and R¹, R⁴, R⁵, Q¹, and Q² are previously defined.

30. A compound of claim 29 having formula (IVa) 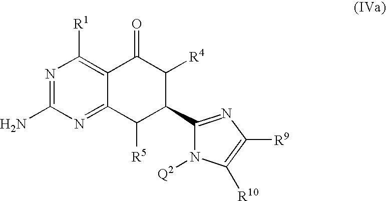
wherein R⁹ and R¹⁰ are independently Q¹, and R¹, R⁴, R⁵, Q¹, and Q² are previously defined.

31. A compound or stereoisomer, tautomer, or pharmaceutically acceptable salt thereof selected from Tables I and II.

32. A composition comprising a pharmaceutically acceptable carrier and a compound having formula (V) 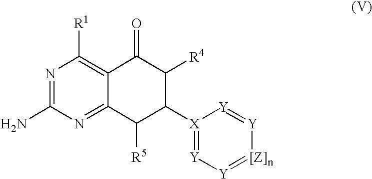
or a stereoisomer, tautomer, or pharmaceutically acceptable salt thereof, wherein
n is 0 or 1;
wherein when n is 1, X is C, Y is at each position independently selected from CQ¹ and N, and Z is selected from CR² and N, and
wherein when n is 0, X is C or N, Y is at each position independently selected from CQ¹, N, NQ², O, and S;
wherein each Q¹ is independently selected from the group consisting of
(1) hydrogen,
(2) halogen,
(3) substituted or unsubstituted C₁-C₆ alkyl,
(4) substituted or unsubstituted C₂-C₆ alkenyl,
(5) substituted or unsubstituted C₂-C₆ alkynyl,
(6) substituted or unsubstituted C₃-C₇ cycloalkyl,
(7) substituted or unsubstituted C₅-C₇ cycloalkenyl,
(8) substituted or unsubstituted aryl,
(9) substituted or unsubstituted heteroaryl,
(10) substituted or unsubstituted heterocyclyl,
(11) substituted or unsubstituted amino,
(12) —OR³, —SR³, or —N(R³)₂,
(13) —C(O)R³, —CO₂R³, —C(O)N(R³)₂, —S(O)R³, —SO₂R³, or —SO₂N(R³)₂,
(14) —OC(O)R³, —N(R³)C(O)R³, or —N(R³)SO₂R³,
(15) —CN, and
(16) —NO₂;
wherein each Q² is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl, and
(9) substituted or unsubstituted heterocyclyl;
wherein R¹ is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C₁-C₆ alkyl,
(8) amino, alkylamino, arylamino, or aralkylamino,
(9) substituted or unsubstituted aryl,
(10) substituted or unsubstituted heteroaryl, and
(11) substituted or unsubstituted heterocyclyl;
wherein R² is selected from the group consisting of
(1) hydrogen,
(2) halogen,
(3) substituted or unsubstituted C₁-C₆ alkyl, and
(4) —OR³, —SR³, or —N(R³)₂;
wherein R⁴ and R⁵ are independently selected from the group consisting of
(1) hydrogen,
(2) halogen,
(3) substituted or unsubstituted C₁-C₆ alkyl,
(4) —OR³, —SR³, or —N(R³)₂, and
(5) —OC(O)R³, —N(R³)C(O)R³, or —N(R³)SO₂R³;
wherein each R³ is independently selected from the group consisting of
(1) hydrogen,
(2) substituted or unsubstituted C₁-C₆ alkyl,
(3) substituted or unsubstituted C₂-C₆ alkenyl,
(4) substituted or unsubstituted C₂-C₆ alkynyl,
(5) substituted or unsubstituted C₃-C₇ cycloalkyl,
(6) substituted or unsubstituted C₅-C₇ cycloalkenyl,
(7) substituted or unsubstituted aryl,
(8) substituted or unsubstituted heteroaryl,
(9) substituted or unsubstituted heterocyclyl, and
(10) substituted or unsubstituted amino.

33. The composition of claim 32, further comprising at least one additional agent selected from the group consisting of irinotecan, topotecan, gemcitabine, imatinib, trastuzumab, 5-fluorouracil, leucovorin, carboplatin, cisplatin, taxanes, tezacitabine, cyclophosphamide, vinca alkaloids, geftinib, vatalanib, sunitinib, sorafenib, erlotinib, dexrazoxane, anthracyclines, and rituximab.

34. A method for treating a condition by modulating HSP90 activity comprising administering to a human or animal subject in need of such treatment an effective amount of a composition of claim 32.

35. The method of claim 34, wherein the condition is cancer.
